(19) United States
(12) Patent Application Publication
(10) Pub. No.: US 2016/0106844 A1
(43) Pub. Date: Apr. 21, 2016

(54) ALTERNATIVE FORMULATIONS FOR TNFR: FC FUSION POLYPEPTIDES

(71) Applicant: MABXIENCE, S.A., Montevideo (UY)

(72) Inventors: Carlos Bañado, Madrid (ES); Tamal Raha, Caranzalem (IN); Cédric Bes, Madrid (ES)

(21) Appl. No.: 14/787,933

(22) PCT Filed: Apr. 29, 2014
(86) PCT No.: PCT/EP2014/058695

§ 371 Date: Oct. 29, 2015

(30) Foreign Application Priority Data
May 2, 2013 (EP) 13166228.0
May 2, 2013 (EP) 13166230.6
Aug. 13, 2013 (EP) 13180169.8

(51) Int. Cl. A61K 47/26; A61K 47/02; A61K 47/12; A61K 38/17

(52) U.S. Cl. CPC A61K 47/26; A61K 38/1793; A61K 47/02; A61K 47/12

(57) ABSTRACT

The present invention relates to aqueous stable pharmaceutical compositions suitable for storage of polypeptides that contain TNFR:Fc.

### FIELD OF INVENTION

The present invention relates to aqueous stable pharmaceutical compositions free of some selected amino acids suitable for storage of polypeptides that contain TNFR:Fc.

### BACKGROUND OF THE INVENTION

Therapeutic polypeptide preparations are often stored prior to use. Polypeptides, however, are unstable if stored in aqueous form for extended period of time, particularly in the absence of a stabilizing agent such as arginine. An alternative to relying on aqueous storage is to prepare a dry lyophilized form of a polypeptide, although, reconstitution of a dried polypeptide often results in aggregation or denaturation. This aggregation of polypeptides is undesirable as it may result in immunogenicity.

A commercially available soluble form of the TNF (tumor necrosis factor) receptor fused to an Fc domain (TNFR:Fc) is known as etanercept. Etanercept (trade name ENBREL®) interferes with tumor necrosis factor (TNF) by acting as a TNF inhibitor. This dimeric fusion polypeptide consisting of the extracellular ligand-binding portion of the human 75 kDa (p75) tumor necrosis factor receptor (TNFR) linked to the Fc portion of human IgG1 is currently formulated with L-arginine and/or L-cysteine as aggregation inhibitor to prevent aggregation of the polypeptide (see EP1478394 B1).

Nevertheless, arginine can cause serious side effects in some people. A severe allergic reaction, called anaphylaxis, can occur after arginine injections, as well as stomach discomfort, including nausea, stomach cramps or an increased number of stools. Other potential side effects include low blood pressure and changes in numerous chemicals and electrolytes in the blood, such as high potassium, high chloride, low sodium, low phosphate, high blood urea nitrogen and high creatinine levels. In theory, arginine may increase the risk of bleeding increase blood sugar levels, increase potassium levels and may worsen symptoms of sickle cell disease.

Cysteine is a non-essential amino acid and is closely related to cystine, as cystine consists of two cysteine molecules joined together. It is an unstable nutrient and is easily converted to cystine. Too much cystine in the body can cause cystinosis, a rare disease that can cause cystine crystals to form in the body and produce bladder or kidney stones. It is also known that people suffering from diabetes and cystinuria may have side-effects with cysteine supplements.

WO2013/006454 discloses arginine-free polypeptide-containing compositions wherein the arginine used in similar compositions as that disclosed in EP1478394 B1 has been replaced with salts, which according to the example provided is 140 mM (see example 1). No reference is made to stabilization at high temperatures. Indeed, the compositions disclosed therein are stored as a liquid at 2-8° C. or frozen.

The present invention addresses these problems by providing a novel stable liquid formulation that allow storage of TNFR:Fc polypeptides. The inventors, surprisingly, have observed that stable aqueous compositions as disclosed herein can be prepared completely free of Arginine and Cysteine and are highly stable at high temperatures.

### SUMMARY OF THE INVENTION

### First Aspect of the Present Invention

The first aspect of the present invention is based on the finding that a certain amount of salt in an aqueous formulation comprising an isolated polypeptide that is an extracellular ligand-binding portion of a human p75 tumor necrosis factor receptor fused to the Fe region of a human IgG1, can result in an increase of stability of the protein at high temperatures, above 5° C. Furthermore, the election of the salt concentration is such that it is close to the physiological body salt concentration.

Therefore, the present invention relates to an aqueous composition comprising:
an isolated polypeptide that is an extracellular ligand-binding portion of a human p75 tumor necrosis factor receptor fused to the Fc region of a human IgG1;
salt present at a concentration of from 80 to 130 mM; and
an excipient selected from the group of trehalose and sucrose and combinations thereof,
   characterized in that neither arginine nor cysteine are present in the composition.

### BRIEF DESCRIPTION OF THE DRAWINGS

FIG. 1 shows a bar chart showing relative unfolding temperatures (Tₒₙₛₑₜ/° C.) found for all samples with error bars found using the fluorescence ratio between 330 and 310 nm.

FIGS. 2A and 2B show a bar chart with measures of pH and osmolality at initial time for all formulations.

FIG. 3A shows the protein concentration measures (Absorbance at 280 nm) at all times (from 0 to 14 days) and conditions (−20° C., 25° C., 50° C., 3 times freezing/thawing (−20° C./25° C.) and 3 days in agitation).

FIG. 3B shows the protein concentration measures (Absorbance at 280 nm) at times up to 6 months (0, 1, 3 and 6) and conditions (−20° C., 2-8° C., 25° C., 1, 2 and 4 times freezing/thawing (−20° C./25° C.)) for formulation F3.

FIG. 4A shows turbidity measures (Absorbance at 330 am) at all times (from 0 to 14 days) and conditions (−20° C., 25° C., 50° C., 3 times freezing/thawing (−20° C./25° C.) and 3 days in agitation).

FIG. 4B(1) shows turbidity measures (Absorbance at 330 nm) at times up to 6 months (0, 1, 3 and 6) and conditions (−20° C., 2-8° C., 25° C., 1, 2 and 4 times freezing/thawing (−20° C./25° C.)) for formulation F3.

FIG. 4B(2) shows turbidity measures (Absorbance at 330 nm) at times up to 3 months (0, 1 and 3) and conditions (−20° C., 2-8° C., 25° C., 1, 2 and 4 times freezing/thawing (−20° C./25° C.)) for formulations F1, F5, F6 and F8 compared to Innovator (t=0 and 3 months and at 25° C.).

FIG. 5A shows sub-visible particle analysis by HIAC for F1, F2, F3 and F4 (1, 2, 3 and 4) measured at all conditions: −20° C., 25° C., 50° C., 3 times freezing/thawing (−20° C./25° C.) and 3 days in agitation using the Standards-Duke Scientific Count Cal.

FIG. 5B shows sub-visible particle analysis by HIAC for formulation F3 measured at t=0, 1 and 3 months and at −20° C., 2-8° C., 25° C., 1 and 2 times freezing/thawing (1× and 2×FzTh at −20° C./25° C.) using the Standards-Duke Scientific Count Cal.

FIG. 5C(1) shows sub-visible particle analysis by HIAC measured for formulations F1, F3, F5, F6, and F8, at t=0, 1 and 3 months, and F3 also at t=6 months, at −20° C. and 2-8° C. using the Standards-Duke Scientific Count Cal.

FIG. 5C(2) shows sub-visible particle analysis by HIAC measured for formulations F1, F3, F5, F6, and F8, at t=0, 1 and 3 months, and F3 also at t=6 months, at 25° C., and freezing/thawing (1×, 2×, 4× (1, 2, 4)) at −20° C./25° C. for F1, F3, F5, F6, and F8.

FIG. 6A shows SDS-PAGE gels stained with Coomassie incubated at all conditions: −20° C., 25° C., 50° C., 3 times freezing/thawing (−20° C./25° C.) and 3 days in agitation at times 0 and 14 days. In (A), F1 sample, in (B) F2 sample, in (C) F3 sample and in (D) F4 sample.

FIG. 6B(1) shows SDS-PAGE gels stained with Coomassie for formulation F3 at t=3 months incubated at all conditions: −20° C., 2-8° C., 25° C., 2 times freezing/thawing at −20° C./25° C.

FIG. 6B(2) shows SDS-PAGE gels stained with Coomassie for formulation F3 at t=6 months incubated at all conditions: −20° C., 2-8° C., 25° C., 4 times freezing/thawing at −20° C./25° C.

FIG. 6C shows SDS-PAGE gels stained with Coomassie for formulations F5, F6 and F7 and Innovator (control) at t=0 and after 1 time freezing/thawing at −20° C./25° C. condition.

FIG. 6D shows SDS-PAGE gels stained with Coomassie for formulations F8, F9 and F1 and Innovator (control) at t=0 and after 1 time freezing/thawing at −20° C./25° C. condition.

FIG. 6E(1) shows SDS-PAGE gels stained with Coomassie for formulations F1 and F5 at t=1 month at −20° C., 2-8° C. and 25° C. and after 2 cycles freezing/thawing at −20° C./25° C. condition.

FIG. 6E(2) shows SDS-PAGE gels stained with Coomassie for formulations F1 and F5 at t=3 months at −20° C., 2-8° C. and 25° C. and after 4 cycles freezing/thawing at −20° C./25° C. condition.

FIG. 6F(1) shows SDS-PAGE gels stained with Coomassie for formulations F6 and F8 at t=1 month at −20° C., 2-8° C. and 25° C. and after 2 cycles freezing/thawing at −20° C./25° C. condition.

FIG. 6F(2) shows SDS-PAGE gels stained with Coomassie for formulations F6 and F8 at t=3 month at −20° C., 2-8° C. and 25° C. and after 4 cycles freezing/thawing at −20° C./25° C. condition.

FIGS. 7A-7D shows the chromatograms of size exclusion HPLC in all formulations for all conditions: −20° C. (7A), 25° C. (7B), 50° C. (7C), 3 times freezing/thawing and 3 days in agitation (7D) at all timepoints. The peak percentages have been measured and represented in the tables.

FIG. 7E(1) shows the chromatogram of size exclusion HPLC in formulation F3 for t=3 months at −20° C., 2-8° C., 25° C. and 2 times freezing/thawing (2×FxTh) at −20° C./25° C. conditions.

FIG. 7E(2) shows the chromatogram of size exclusion HPLC in formulation F3 for t=6 months at −20° C., 2-8° C., 25° C. and 4 times freezing/thawing (2×FxTh) at −20° C./25° C. conditions.

FIG. 7F shows the chromatogram of size exclusion HPLC in formulation F3 for t=0, 1, 3 and 6 months at 25° C. and Innovator at t=3 months and 25° C.

FIG. 7G(1) shows the chromatogram of size exclusion HPLC in formulation F3 for t=0 and 3 months at 25° C. and compared to Innovator (control) at t=0.

FIG. 7G(2) shows the chromatogram of size exclusion HPLC in formulation Innovator for t=0 and 3 months at 25° C.

FIG. 7H provides the tabular results for a longer term study with size exclusion HPLC in formulation F3 for t=0, 1 and 3 months at −20° C., 2-8° C., 25° C. and 1 and 2 times freezing/thawing (1× and 2×FxTh) at −20° C./25° C. conditions.

FIG. 7I shows the chromatogram of size exclusion HPLC in formulations F1, F5, F6, F7, F8, F9 and Innovator (control) at t=0.

FIG. 7J shows the chromatogram of size exclusion HPLC in formulations F1, F5, F6, F7, F8, F9 and Innovator after 1 cycle freezing/thawing at −20° C./25° C.

FIG. 7K(1) shows the chromatogram of size exclusion HPLC in formulations F1, F5, F6, F8, for t=1 month at −20° C.

FIG. 7K(2) shows the chromatogram of size exclusion HPLC in formulations F1, F3, F5, F6 and F8, for t=3 months at −20° C.

FIG. 7L(1) shows the chromatogram of size exclusion HPLC in formulations F1, F5, F6, F8, for t=1 month at 2-8° C.

FIG. 7L(2) shows the chromatogram of size exclusion HPLC in formulations F1, F3, F5, F6 and F8, for t=3 month at 2-8° C.

FIG. 7M(1) shows the chromatogram of size exclusion HPLC in formulations F1, F5, F6, F8, for t=1 month at 25° C.

FIG. 7M(2) shows the chromatogram of size exclusion HPLC in formulations F1, F3, F5, F6, F8 and Innovator for t=3 month at 25° C.

FIG. 7N(1) shows the chromatogram of size exclusion HPLC in formulations F1, F5 and F8, for t=1 month at 25° C.

FIG. 7N(2) shows the chromatogram of size exclusion HPLC in formulations F1, F3, F5, F8 and Innovator for t=3 month at 25° C.

FIG. 7O shows the chromatogram of size exclusion HPLC in formulations F1, F3, F5 and F8, for t=1 month at 25° C.

FIG. 7P shows the chromatogram of size exclusion HPLC in formulations F1, F5, F6 and F8 after 2 cycles freezing/thawing at −20° C./25° C.

FIGS. 7Q, 7R and 7S show the graphical summary of chromatograms of size exclusion HPLC in formulations F1, F3, F5, F6 and F8 for conditions: −20° C. (FIG. 7Q), 2-8° C. (7R) and 25° C. (7S) at timepoints up to 6 months for formulation F3 and up to 3 month for formulations F1, F5, F6 and F8. The peak percentages have been measured and represented (% pre-peak, % main-peak and % post-peak)

FIG. 7T shows the graphical summary of chromatograms of size exclusion HPLC in formulations F1, F3, F5, F6 and F8 at t=0 and after 1 and 2 cycles freezing/thawing (1× and 2×FxTh) at −20° C./25° C. conditions. The peak percentages have been measured and represented (% pre-peak, % main-peak and % post-peak). Bars are indicated in the following order of formulation: F1, F3, F5, F6 and F8 for each condition (i.e. t=0, 1×FxTh or 2×FxTh).

FIG. 7U shows the graphical summary of chromatograms of size exclusion HPLC in formulation F3 for t=0, 1, 3, and 6 months at −20° C., 2-8° C. and 25° C. storage conditions.

FIG. 8A-8D shows a graph including the analysis of a cell based potency assay (% of relative potency, as compared to potency of the reference standard) in all formulations for all conditions: −20° C. (8A), 25° C. (8B), 50° C. (8C), 3 times freezing/thawing (−20° C./25° C.) and 3 days in agitation (8D) at all timepoints.

FIG. 8E shows a graph including the analysis of a cell based potency assay (% of relative potency, as compared to potency of the reference standard) in formulation F3 for the following conditions: −20° C., 2-8° C., 25° C. at time 0, 1, 3, and 6 months, and after 1×, 2× and 4× freezing/thawing at −20° C./25° C. The data table is also provided next to the figure.

FIG. 8F shows a graph including the analysis of a cell based potency assay (% of relative potency, as compared to potency of the reference standard) in formulations F1, F3, F5, F6 and F8 after 3 month (and F3 also after 6 months) at −20° C., 2-8° C., 25° C. and after 4× freezing/thawing at −20° C./25° C., compared to Innovator after 3 months at 25° C. The data table is also provided next to the figure.

### DETAILED DESCRIPTION OF THE INVENTION

The present invention relates to an aqueous composition comprising:
an isolated polypeptide that is an extracellular ligand-binding portion of a human p75 tumor necrosis factor receptor fused to the Fc region of a human IgG1;
salt present at a concentration of from 80 to 130 mM; and
an excipient selected from the group consisting of trehalose and sucrose and combinations thereof,
   characterized in that neither arginine nor cysteine are present in the composition.

Preferably, the composition is further characterized in that no free amino acids are present in the composition. For example, the composition neither comprises arginine, nor cysteine, nor proline, nor glycine, nor methionine, nor histidine, nor serine, nor valine, nor lysine, nor glutamate.

As used herein, the term “composition” or “compositions” may refer to a formulation(s) comprising a polypeptide prepared such that it is suitable for injection and/or administration into an individual in need thereof. A “composition” may also be referred to as a “pharmaceutical composition.” In certain embodiments, the compositions provided herein are substantially sterile and do not contain any agents that are unduly toxic or infectious to the recipient. Further, as used herein, a solution or aqueous composition may mean a fluid (liquid) preparation that contains one or more chemical substances dissolved in a suitable solvent (e.g., water and/or other solvent, e.g., organic solvent) or mixture of mutually miscible solvents. Further, as used herein, the term “about” means the indicated value±2% of its value, preferably the term “about” means exactly the indicated value (±0%).

Note that although the composition according to the present invention does not comprise arginine or cysteine (or, preferably, any other amino acid such as proline, glycine, methionine, histidine, serine, valine, lysine, glutamate) alone or added to the composition, the polypeptide itself can contain arginine or cysteine (or any other amino acid such as proline, glycine, methionine, histidine, serine, valine, lysine, glutamate) amino acid residues in its chain.

In certain embodiments, the expressed Fc domain containing polypeptide is purified by any standard method. When the Fc domain containing polypeptide is produced intracellularly, the particulate debris is removed, for example, by centrifugation or ultrafiltration. When the polypeptide is secreted into the medium, supernatants from such expression systems can be first concentrated using standard polypeptide concentration filters. Protease inhibitors can also be added to inhibit proteolysis and antibiotics can be included to prevent the growth of microorganisms. In some embodiments, the Fc domain containing polypeptide is purified using, for example, hydroxyapatite chromatography, gel electrophoresis, dialysis, and affinity chromatography, and/or any combination of purification techniques known or yet to discovered. For example, protein A can be used to purify Fc domain containing polypeptides that are based on human gamma 1, gamma 2, or gamma 4 heavy chains (Lindmark et al., 1983, J. Immunol. Meth. 62: 1-13).

Other techniques for polypeptide purification such as fractionation on an ion-exchange column, ethanol precipitation, reverse phase HPLC, chromatography on silica, chromatography on heparin SEPHAROSET™, chromatography on an anion or cation exchange resin (such as a polyaspartic acid column), chromatofocusing, SDS-PAGE, and ammonium sulfate precipitation can also be utilized depending on the needs. Other polypeptide purification techniques can be used.

In a preferred embodiment, the salt concentration is from 80 to 130 mM, preferably from 90 to 130 mM, such as from 105 to 130 mM, such as about 90 mM, 100 mM or 125 mM. Preferably, the salt concentration (preferably NaCl) is about 90 mM. Regardless of the concentration, the salt is preferably sodium chloride, although other salts such as potassium chloride, sodium citrate, magnesium sulphate, calcium chloride, sodium hypochlorite, sodium nitrate, mercury sulphide, sodium chromate and magnesium dioxide can also be used. This particular range of salt concentrations allows obtaining a composition according to the present invention which is stable at high temperatures, even up to 50° C. In addition, the values in this range are closer to the physiological osmolality in the human body than those values used in prior art (e.g. 140 mM), leading to more suitable compositions to be used in e.g. subcutaneous administration.

In another preferred embodiment, the isolated polypeptide is etanercept. The Fc component of etanercept contains the constant heavy 2 (CH2) domain, the constant heavy 3 (CH3) domain and hinge region, but not the constant heavy 1 (CH1) domain of human IgG1. Etanercept may be produced by recombinant DNA technology in a Chinese hamster ovary (CHO) mammalian cell expression system. It consists of 934 amino acids and has an apparent molecular weight of/approximately 150 kilodaltons (Physicians' Desk Reference, 2002, Medical Economics Company Inc.).

The concentration of the isolated polypeptide is preferably from 10 to 100 mg/mL, more preferably between 20 and 60 mg/mL and even more preferably the concentration is about 25 mg/mL or about 50 mg/mL. Preferably, the concentration is about 50 mg/mL.

In another preferred embodiment, the excipient is trehalose at a concentration from 10 to 80 mg/mL, preferably from 30 to 65 mg/mL and more preferably at a concentration of 60 mg/mL of trehalose and in the form of trehalose dihydrate. In another preferred embodiment, the excipient is sucrose at a concentration from 5 to 80 mg/mL, preferably sucrose is present in the range of 10 to 40 mg/mL. In a more preferred embodiment the concentration of sucrose is 10 mg/mL. In another more preferred embodiment, the concentration of sucrose is 34 mg/mL. In another preferred embodiment, the excipient is a combination between sucrose and trehalose, where the concentrations are in the range of 5 to 80 mg/mL and 10 to 80 mg/mL, respectively. Preferably, the excipient is sucrose at a concentration of about 34 mg/mL. More preferably, the excipient is sucrose at a concentration of about 10 mg/mL.

The composition according to the present invention may further comprise an aqueous buffer. Preferably, said aqueous buffer is sodium phosphate, potassium phosphate, sodium or potassium citrate, maleic acid, ammonium acetate, tris-(hydroxymethyl)-aminomethane (tris), acetate, succinate, diethanolamine, histidine or a combination thereof. In a more preferred embodiment said aqueous buffer is sodium phosphate. In another more preferred embodiment said aqueous buffer is succinate. In another more preferred embodiment said aqueous buffer is histidine. Regardless of the buffer used in the composition, alone or in combination, the concentration thereof is preferably between 15 mM and 100 mM, preferably in the range of 20 mM to 30 mM. In a preferred embodiment said concentration is preferably between 20 mM and 100 mM, preferably in the range of 25 mM to 50 mM. In a more preferred embodiment said concentration is about 22 mM or about 25 mM. In another preferred embodiment said concentration is about 50 mM. Preferred buffers are sodium phosphate and succinate buffer, being this last one (succinate buffer) in a concentration of about 22 mM the most preferred one.

In another embodiment, regardless of the absence or the presence of the aqueous buffer, the composition according to the present invention may further comprise one or more excipients, in addition to the one already provided in the composition (trehalose or sucrose). In certain embodiments, the concentration of one or more excipients in the composition described herein is about 0.001 to 5 weight percent, while in other embodiments; the concentration of one or more excipients is about 0.1 to 2 weight percent. Excipients are well known in the art and are manufactured by known methods and available from commercial suppliers. Preferably, said excipient is lactose, glycerol, xylitol, sorbitol, mannitol, maltose, inositol, glucose, bovine serum albumin, human serum albumin (SA), recombinant hemagglutinin (HA), dextran, polyvinyl alcohol (PVA), hydroxypropyl methylcellulose (HPMC), polyethylenimine, gelatine, polyvinylpyrrolidone (PVP), hydroxyethylcellulose (HEC), polyethylene glycol, ethylene glycol, dimethysulfoxide (DMSO), dimethylformamide (DMF), proline, L-serine, glutamic acid, alanine, glycine, lysine, sarcosine, gamma-aminobutyric acid, polysorbate 20, polysorbate 80, sodium dodecyl sulfate (SDS), polysorbate, polyoxyethylene copolymer, potassium phosphate, sodium acetate, ammonium sulphate, magnesium sulphate, sodium sulphate, trimethylamine N-oxide, betaine, zinc ions, copper ions, calcium ions, manganese ions, magnesium ions, 3-[(3-cholamidepropyl)-dimethylammonio]-1-propanesulfate (CHAPS), sucrose monolaurate or a combination thereof. In a more preferred embodiment, the excipient is polysorbate 20 and in an even more preferred embodiment the polysorbate 20 is present at a concentration of 0.1%. In another more preferred embodiment, the excipient is glycine and in an even more preferred embodiment glycine is present at a concentration of 0.5%.

In another preferred embodiment, the pH of the composition is from pH 6.0 to pH 7.0, being possible any pH selected from 6.1, 6.2, 6.3, 6.4, 6.5, 6.6, 6.7, 6.8 and 6.9. In a more preferred embodiment, the pH of the composition is about 6.3.

In a particular embodiment, the composition according to the present invention comprises 50 mg/mL of etanercept, 25 mM sodium phosphate buffer, 10 mg/mL sucrose, 125 mM sodium chloride, wherein the pH of the composition is 6.3.

In another particular embodiment, the composition according to the present invention comprises 50 mg/mL of etanercept, 25 mM sodium phosphate buffer, 10 mg/mL sucrose, 100 mM sodium chloride, wherein the pH of the composition is 6.3.

In another particular embodiment, the composition according to the present invention comprises 50 mg/mL of etanercept, 50 mM sodium phosphate buffer, 60 mg/mL trehalose dihydrate, 0.1% Polysorbate 20, wherein the pH of the composition is about pH 6.2.

In a further particular embodiment, the composition according to the present invention comprises 50 mg/mL of etanercept, 25 mM sodium phosphate, 34 mg/mL sucrose, 90 mM sodium chloride, wherein the pH of the composition is 6.3.

In a further particular embodiment, the composition according to the present invention comprises 50 mg/mL of etanercept, 25 mM sodium phosphate, 10 mg/mL sucrose, 90 mM sodium chloride, 0.5% glycine, wherein the pH of the composition is 6.3.

In a further particular embodiment, the composition according to the present invention comprises 50 mg/mL of etanercept, 22 mM succinate, 10 mg/mL sucrose, 90 mM sodium chloride, wherein the pH of the composition is 6.3. Preferably, this composition is free from additional amino acids (apart from the ones comprised in etanercept). Preferably, this composition neither comprises arginine, nor cysteine, nor lysine, nor proline, nor glutamate, nor serine, nor methionine.

The compositions disclosed herein can be administered parenterally, e.g. subcutaneously, intramuscularly, intravenously, intraperitoneal, intracerebrospinal, intraarticular, intrasynovial and/or intrathecal.

The therapeutic effect of the isolated polypeptide comprised in the compositions according to the present invention are known in the art and includes, but not limited thereto, treating rheumatoid arthritis, psoriatic arthritis, ankylosing spondylitis, granulomatosis, Crohn's disease, chronic obstructive pulmonary disease, hepatitis C, endometriosis, asthma, cachexia, psoriasis or atopic dermatitis, or other inflammatory or autoimmune-related illness, disorder, or condition. The compositions may be administered in an amount sufficient to treat (alleviate symptoms, halt or slow progression of) the disorder (e.g., a therapeutically effective amount).

The following examples serve to illustrate the present invention and should not be construed as limiting the scope thereof.

### EXAMPLES

### Preparation of Compositions

The following compositions were prepared by simple mixing:

### Source Material:

Engineering Run Material containing 62.5 mg/mL of etanercept, 1.2 mg/mL Tris, 40 mg/mL Mannitol, 10 mg/mL Sucrose, pH 7.4. Stored at −20° C.

A lot of Enbrel® commercial formulation was used as a control sample (designated herein as “Enbrel” or “Innovator”). The commercial Enbrel formulation contains 50 mg/mL etanercept, 25 mM Na phosphate, 25 mM Arginine, 100 mM NaCl, 10 mg/mL Sucrose, pH 6.3).

Etanercept in the same formulation as Enbrel formulation was used as internal control (50.9 mg/mL etanercept, 25 mM Na phosphate, 25 mM Arginine, 100 mM NaCl, 10 mg/mL Sucrose, pH 6.3). This formulation was called F1.

### Candidate Formulations:

F2: Etanercept in aqueous formulation (49.4 mg/mL etanercept, 25 mM Na phosphate, 100 mM NaCl, 10 mg/mL Sucrose, pH 6.3)

F3: Etanercept in aqueous formulation (49.5 mg/mL etanercept, 25 mM Na phosphate, 125 mM NaCl, 10 mg/mL Sucrose, pH 6.3)

F4: Etanercept in aqueous formulation (50.9 mg/mL etanercept, 50 mM Na phosphate, 60 mg/mL Trehalose dihydrate, pH 6.2, 0.1% Polysorbate 20)

F5: Etanercept in aqueous formulation (50.0 mg/mL etanercept, 25 mM Na phosphate, 90 mM NaCl, 34 mg/mL Sucrose, pH 6.3)

F6: Etanercept in aqueous formulation (50.0 mg/mL etanercept, 25 mM Na phosphate, 90 mM NaC, 10 mg/mL Sucrose, 0.5% (5 mg/mL) glycine, pH 6.3)

F7: Etanercept in aqueous formulation (50.0 mg/mL etanercept, 28 mM Histidine/HCl, 90 mM NaCl, 10 mg/mL Sucrose, 6 mg/mL glycine, pH 6.3)

F8: Etanercept in aqueous formulation (50.0 mg/mL etanercept, 22 mM succinate, 90 mM NaCl, 10 mg/mL Sucrose, pH 6.3). Succinate buffer was prepared using succinic acid 22 mM and NaOH was added to adjust pH to 6.3.

### Example 1

### Intrinsic Protein Fluorescence Emission Spectra and Static Light Scattering

Intrinsic protein fluorescence emission spectra, excited at 266 am, were acquired as well as static light scattering data at both 266 and 473 nm. Each sample was loaded into a micro-cuvette array (MCA) and placed into the Optim 1000 to elucidate differences in colloidal and conformational stabilities. In this study the temperature for thermal ramp experiments was increased from 15 to 95° C. in 1° C. steps, and samples were held at each temperature for 60 seconds to allow thermal equilibration. In the isothermal experiment, the temperature was held at 62° C. and samples were measured with 200 repeats with a 60 second hold between measurements.

The time during which the sample is illuminated with the 266 and 473 nm laser sources is referred to as the exposure time. The choice of exposure time depends on a number of factors, such as how strong the fluorescence emission is and how susceptible the sample is to photobleaching. In the case of all of these samples, an exposure time of 1 second was used.

Along with changing the exposure time it is possible to change the size of a physical slit which controls the amount of light which enters the detector. Increasing the size of this opening increases the fluorescence signal measured, but decreases the spectral resolution of the instrument.

The analyses performed by the Optim 1000 comprise two sequential levels, primary and secondary. The Optim 1000 software provides automated primary and secondary analysis. As with any automated data fitting software, sensible care must be taken to ensure that the input data is of good quality so that the automated functions return reliable results. All the results have been checked manually by a trained analyst.

The primary analysis extracts spectral parameters from the raw fluorescence emission and light scattering data:
Optim can use mathematical functions to provide primary level information such as expectation wavelength (also called the barycentric mean) which is becoming more commonly used in the scientific literature. This looks at the average emission wavelength (or centre of mass), and is a good approach to smooth out any noise in spectral data.
Scattered light intensity is calculated from the integrated intensity between 260 and 270 am (the Rayleigh scattered UV excitation light). Scattering efficiency is very dependent on wavelength, so the shorter it is the more efficiently that light is scattered by molecules in the solution. The scattering of the 266 nm laser is a very sensitive probe to small changes in mean molecular mass.

In this study, the ratio of fluorescence intensity between 350 and 330 am has been used to study the thermal unfolding of the antibodies and the scattered light intensity from the 266 nm and 473 nm lasers was used to measure thermally induced sample aggregation.

Secondary analysis takes the parameters from the primary analyses and determines the melting temperature “Tₘ” and aggregation onset temperature “T_{agg}” of the sample, if these exist. The melting temperature is determined as the inflection point in the primary data plotted as a function of temperature.

The onset of aggregation temperature is determined as the temperature at which the scattered light intensity increases above a threshold value relative to the noise in the data. From the lowest temperature measured, each scattered intensity value measured is added to a dataset of all previously measured values. At each point, as the analysis progresses, a linear fit is applied and the goodness of the fit determined. If the data deviates significantly from a straight line (where the significance is determined by the noise in the data) then this is defined as the temperature of the onset of aggregation. If it doesn't then the algorithm proceeds to the next point in the dataset and once again tests for this deviation. This method has been tested on a variety of proteins and conditions and is robust. In extreme situations where large aggregates form and precipitate, the light scattering signal can actually fall if the particles in suspension leave the focal volume of the incident laser. However, the initial onset is detected reproducibly despite any precipitation which occurs afterward.

In the case of all static light scattering data, all points have been included regardless of whether the sample appeared to precipitate out of solution. The same sample in different repeated experiments will sometimes precipitate and sometimes not, but in each case the start of the aggregation process is reproducible.

### Conclusions

Both the T_{agg} and Tₒₙₛₑₜ data between all samples were found to be very similar.
In F1 buffer the product was found to have a Tₒₙₛₑₜ of fluorescence of 63.7±0.3° C. and a T_{agg} of 66.8±0.3° C.
In F2 buffer the product was found to have a Tₒₙₛₑₜ of fluorescence of 63.2±0.1° C. and a T_{agg} of 65.9±0.1° C.
In F3 buffer the product was found to have a Tₒₙₛₑₜ of fluorescence of 63.4 t 0.3° C. and a T_{agg} of 65.6±0.4° C.
In F4 buffer the product was found to have a Tₒₙₛₑₜ of fluorescence of 63.3±0.1° C. and a T_{agg} of 64.8±0.1° C.
In F5 buffer the product was found to have a Tₒₙₛₑₜ of fluorescence of 64.5±0.4° C. and a T_{agg} of 63.0±0.6° C.
In F6 buffer the product was found to have a Tₒₙₛₑₜ of fluorescence of 63.9±0.5° C. and a T_{agg} of 65.4±0.2° C.
In F7 buffer the product was found to have a Tₒₙₛₑₜ of fluorescence of 61.0±0.7° C. and a T_{agg} of 63.6±0.1° C.
In F8 buffer the product was found to have a Tₒₙₛₑₜ of fluorescence of 64.0±0.0° C. and a T_{agg} of 66.2±0.8° C.
Enbrel innovator itself was found to have a Tₒₙₛₑₜ of fluorescence of 63.4±0.1° C. and a T_{agg} of 65.6±0.1° C.

The data therefore indicates a high degree of similarity in both colloidal and conformational stability between all samples.

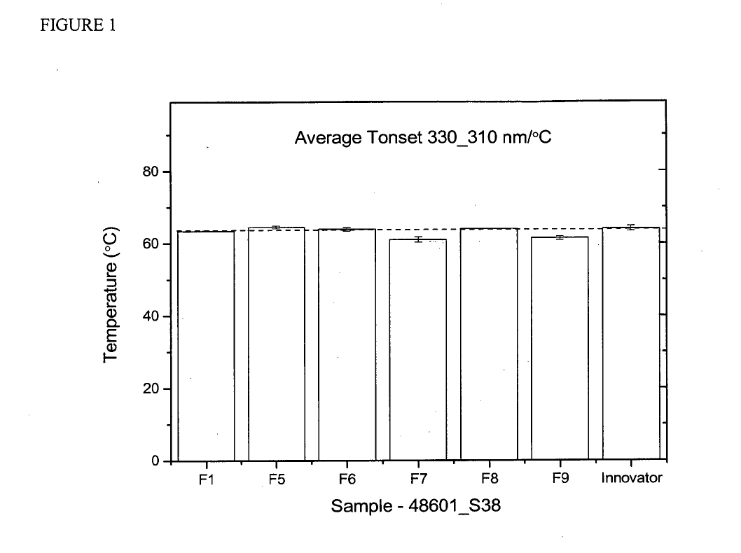

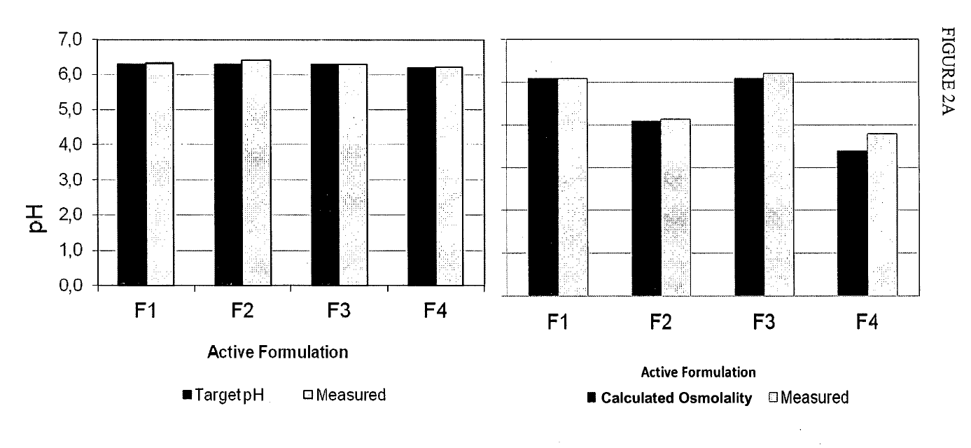

FIG. 1 shows the results for formulations F1, F5, F6, F7, F8 and Innovator (control), where the trend is F5>F8>F6>F1>Enbrel>F7.

Following the thermal ramp experiment an isothermal experiment was performed. After analysis and review of the thermal ramp results, it appeared that all samples had a T_{agg}, value of ˜64° C., and so a temperature of 62° C. was selected for the isothermal experiment, i.e. just below the T_{agg}, but close enough for samples to undergo conformational and colloidal changes within a reasonable time period.

The Tₒₙₛₑₜ values found for fluorescence were between 63.2 and 63.7° C. with a mean of 63.4° C. and a relatively low standard deviation of 0.3° C., indicating a high degree of comparability between the five samples (F1 to F4 and Enbrel-liquid formulation).

The stability of all the samples can still be considered to be fairly comparable.

### Example 2

### Short Stress Stability Study

A short-term (2-week) stability study was performed in order to evaluate possible formulations prior to execution of a longer-term study. Furthermore, a long-term stability study of up to 6 months was performed for F3 formulation and of up to 3 months for F5, F6 and F8 formulations.

Nine formulations were tested:

| | |
|---|---|
| F1 formulation | 25 mM Na phosphate, 25 mM Arginine, 100 |
| | mM NaCl, 10 mg/mL Sucrose, pH 6.3 |
| F2 formulation | 25 mM Na phosphate, 100 mM NaCl, 10 mg/mL |
| | Sucrose, pH 6.3 |
| F3 formulation | 25 mM Na phosphate, 125 mM NaCl, 10 mg/mL |
| | Sucrose, pH 6.3 |
| F4 formulation | 50 mM Na phosphate, 60 mg/mL Trehalose |
| | dihydrate, pH 6.2, 0.1% Polysorbate 20 |
| F5 formulation | 25 mM Na phosphate, 90 mM NaCl, 34 mg/mL |
| | Sucrose, pH 6.3 |
| F6 formulation | 25 mM Na phosphate, 90 mM NaCl, 10 mg/mL |
| | Sucrose, 0.5% (5 mg/mL) glycine, pH 6.3 |
| F7 formulation | 28 mM Histidine/HCl, 90 mM NaCl, 10 mg/mL |
| | Sucrose, 6 mg/mL glycine, pH 6.3 |
| F8 formulation | 22 mM succinate, 90 mM NaCl, 10 mg/mL |
| | Sucrose, pH 6.3 |
| F9 formulation | Internal sample |
| (not part of | |
| the invention) | |

The stability of each formulation at t=0, 3, 7 and 14 days was assessed, following exposure to two elevated temperatures (25° C. and 50° C.) and one real-time temperature, in addition to agitation and freeze-thaw stress.

In the case of F3 formulation, the stability was assessed following exposure to three temperatures (2-8° C., −20° C. and 25° C.) with time points 0, 1, 3 and 6 months in addition to freeze-thaw stress with 1, 2 and 4 freeze-thaw cycles subjected to −20° C. freeze/25° C. thaw.

In the case of F5, F6 and F8 formulations, the stability was also assessed following exposure to three temperatures (2-8° C., −20° C. and 25° C.) with time points 0, 1 and 3 months in addition to freeze-thaw stress with 1, 2 and 4 freeze-thaw cycles subjected to −20° C. freeze/25° C. thaw.

A panel of 8 analytical assays was employed to assess the stability of each formulation.
pH (t=0 only)
Osmolality (t=0 only)
Protein concentration (A280 nm)
Turbidity (A330 nm)
HIAC
SDS-PAGE reduced (coomassie blue stain)
Size Exclusion-HPLC (SE-HPLC)
Cell-based potency

### pH and Osmolality

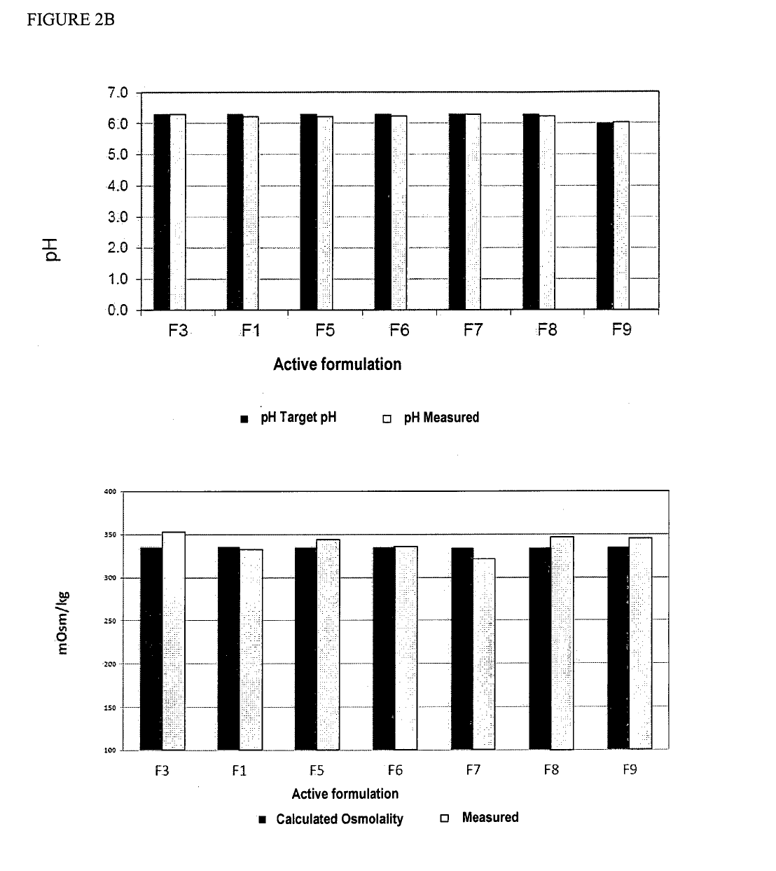

FIGS. 2A and 2B show a bar chart with measures of pH and osmolality at initial time. These values measured for all formulations were within range of target pH or theoretical osmolality value prior to setting up the samples at each of the conditions.

### Protein Concentration/A280

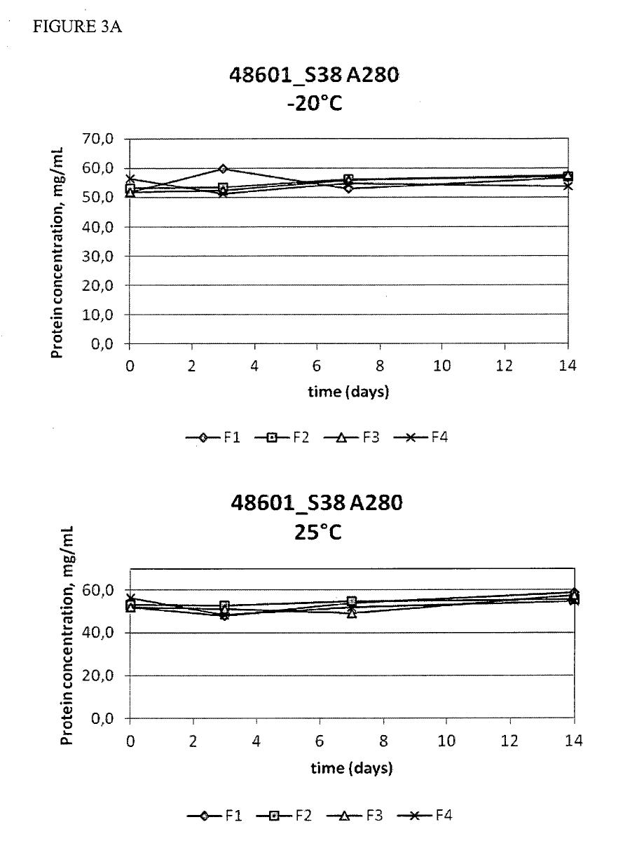

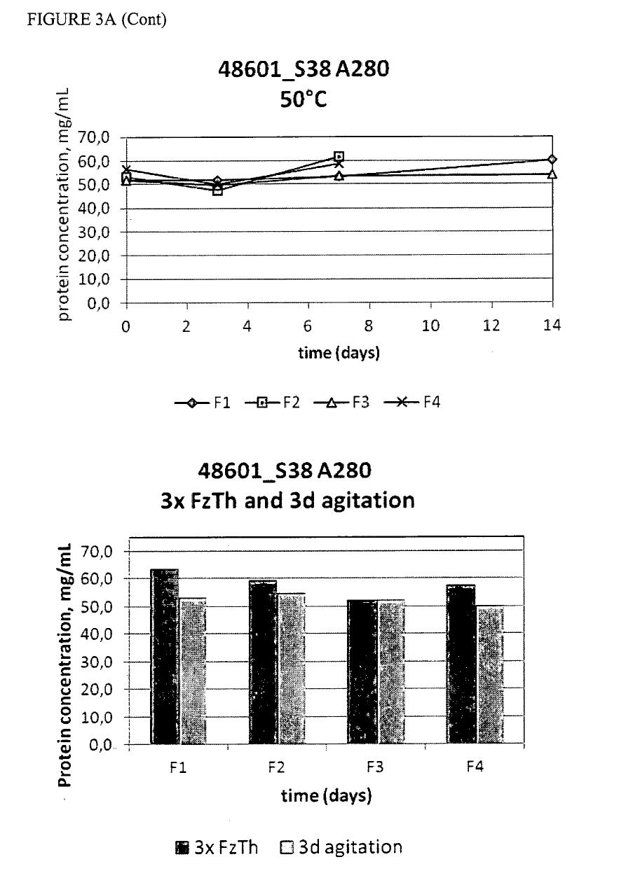

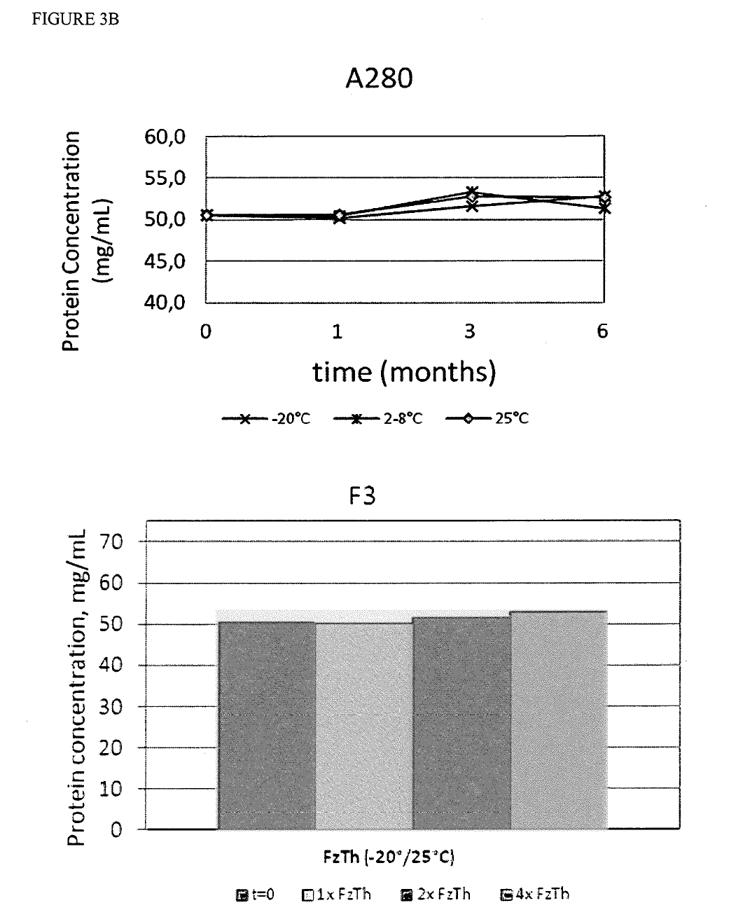

FIG. 3A shows the protein concentration measures (Absorbance at 280 nm) at all times (from 0 to 14 days) and conditions (−20° C., 25° C., 50° C., 3 times freezing/thawing (3×FzTh) and 3 days in agitation). The data obtained remained within range of target value and within variability of the assay for all samples at all timepoints and conditions.

FIG. 3B shows the protein concentration measures for formulation F3 (Absorbance at 280 nm) at times 0, 1, 3 and 6 months and conditions (−20° C., 2-8° C., 25° C., 1, 2 and 4 times freezing/thawing (1×, 2× and 4×FzTh)). A slight increase in protein concentration from target (50 mg/mL) is observed, but still remaining within assay variability for all conditions up to 3 months. Data for constructing said FIG. 3B is provided in the following table:

| | | | | A330, | A280, | |
|---|---|---|---|---|---|---|
| | | Time Point | Dilution | AU | AU | Conc |
| Formulation | Condition | (months) | Factor | Active | Active | (mg/mL) |
| F3 | t = 0 | 0 | 75 | 0.007 | 0.768 | 50.5 |
| | −20° C. | 1 | 75 | 0.005 | 0.762 | 50.2 |
| | | 3 | 75 | 0.005 | 0.812 | 52.0 |
| | | 6 | 75 | 0.001 | 0.803 | 52.8 |
| | 2-8° C. | 1 | 75 | 0.000 | 0.766 | 50.4 |
| | | 3 | 75 | 0.006 | 0.854 | 53.3 |
| | | 6 | 75 | 0.005 | 0.781 | 51.4 |
| | 25° C. | 1 | 75 | 0.006 | 0.769 | 50.6 |
| | | 3 | 75 | 0.005 | 0.819 | 52.8 |
| | | 6 | 75 | 0.002 | 0.802 | 52.7 |
| | Fz Th (−20° C./ | 1x cycle | 75 | 0.005 | 0.762 | 50.1 |
| | 25° C.) | 2x cycle | 75 | 0.003 | 0.798 | 51.6 |
| | | 4x cycle | 75 | 0.002 | 0.804 | 52.9 |

The following table summarizes the data obtained for formulations F1, F5, F6, F8, and Innovator (control, only 25° C. t=0 and t=3) at t=0 and t=3 months at −20° C., 2-8° C. and 25° C., and after 4 cycles of freeze-thaw at −20° C./25° C. The protein concentration is at or close to target (50 mg/mL) for all the formulations.

| | | | Time Point | Protein concen- |
|---|---|---|---|---|
| Formulation | Condition | | (months) | tration, mg/mL |
| F1 | t = 0 Control | | 0 | 50.9 |
| | −20° | C. | 3 | 50.2 |
| | 2-8° | C. | 3 | 50.1 |
| | 25° | C. | 3 | 49.4 |
| | Fz Th (−20° C./25° C.) | | 4x | 48.8 |
| F5 | t = 0 Control | | 0 | 50.2 |
| | −20° | C. | 3 | 49.7 |
| | 2-8° | C. | 3 | 50.5 |
| | 25° | C. | 3 | 49.3 |
| | Fz Th (−20° C./25° C.) | | 4x | 50.0 |
| F6 | t = 0 Control | | 0 | 50.2 |
| | −20° | C. | 3 | 50.1 |
| | 2-8° | C. | 3 | 51.0 |
| | 25° | C. | 3 | 50.0 |
| | Fz Th (−20° C./25° C.) | | 4x | 49.2 |
| F8 | t = 0 Control | | 0 | 51.1 |
| | −20° | C. | 3 | 50.4 |
| | 2-8° | C. | 3 | 49.9 |
| | 25° | C. | 3 | 48.9 |
| | Fz Th (−20° C./25° C.) | | 4x | 47.8 |
| Innovator | 25° | C. | 0 | 48.1 |

| | |
|---|---|
| 3 | 49.1 |

The protein concentration measures for formulations F5, F6 and F8 (Absorbance at 280 nm) at time=3 months remained at target value for all these formulations, in addition to F1, at all conditions (Figure not shown).

### Turbidity/A330

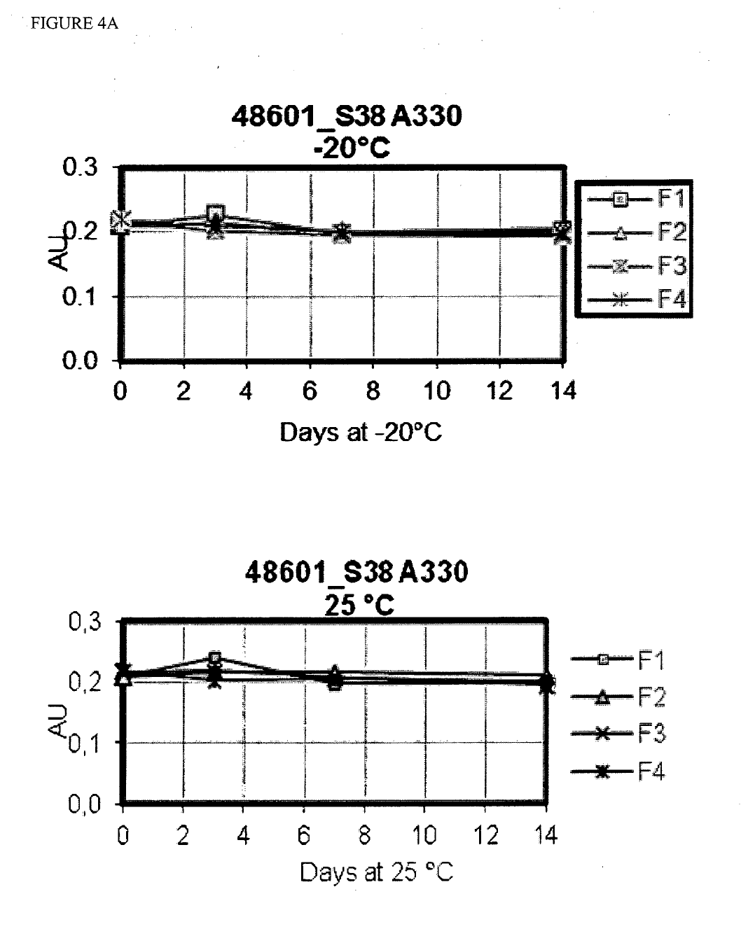

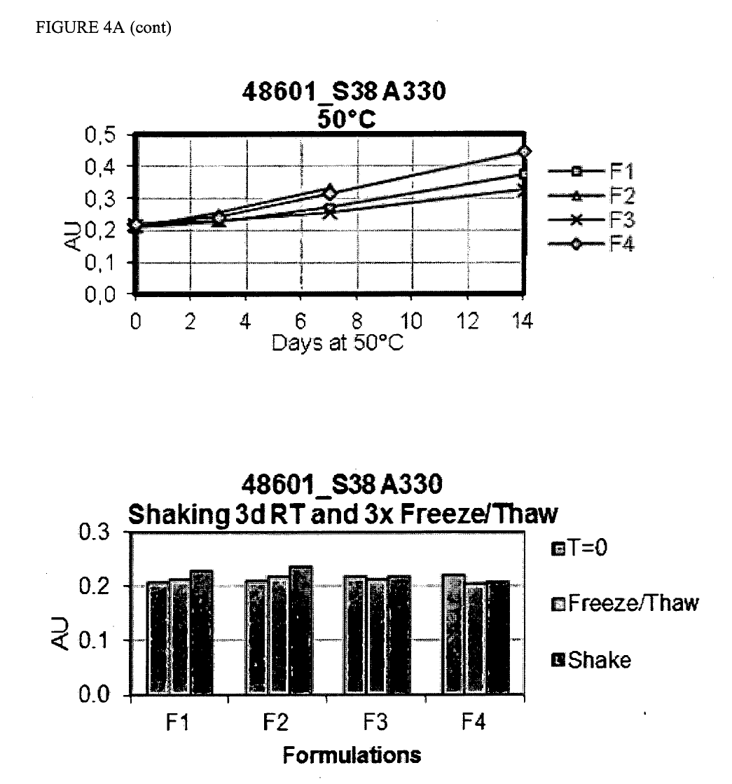

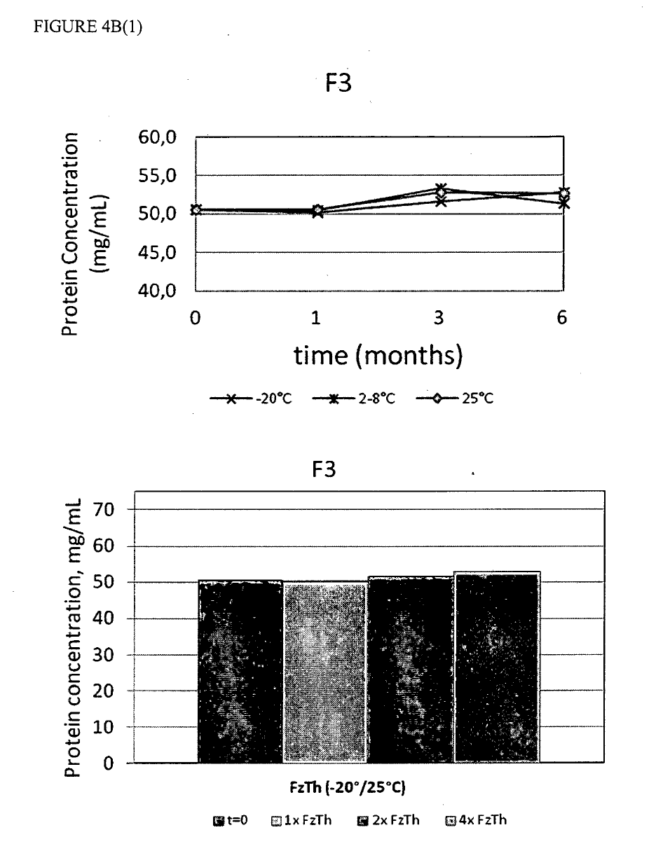

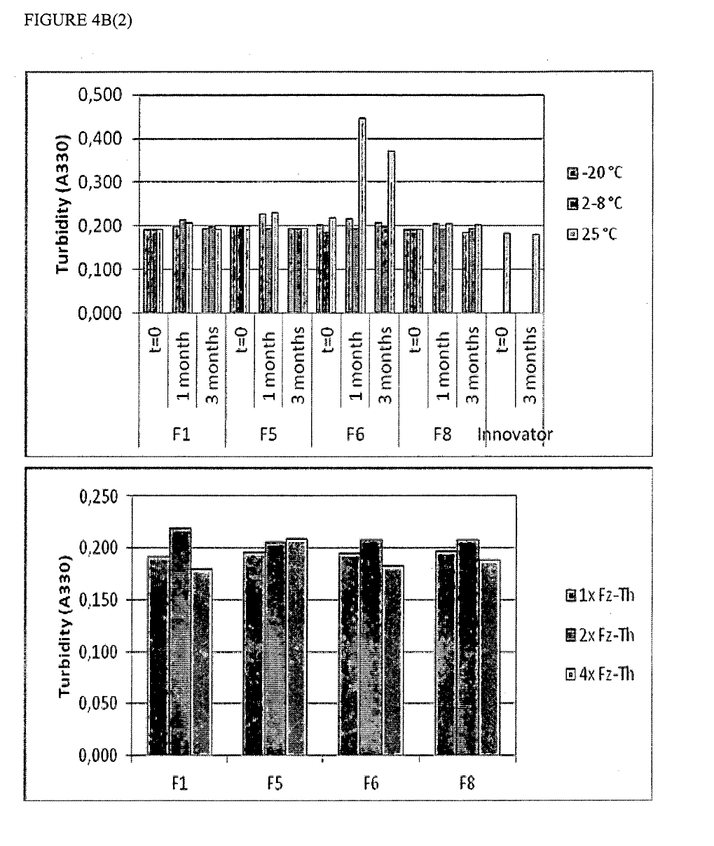

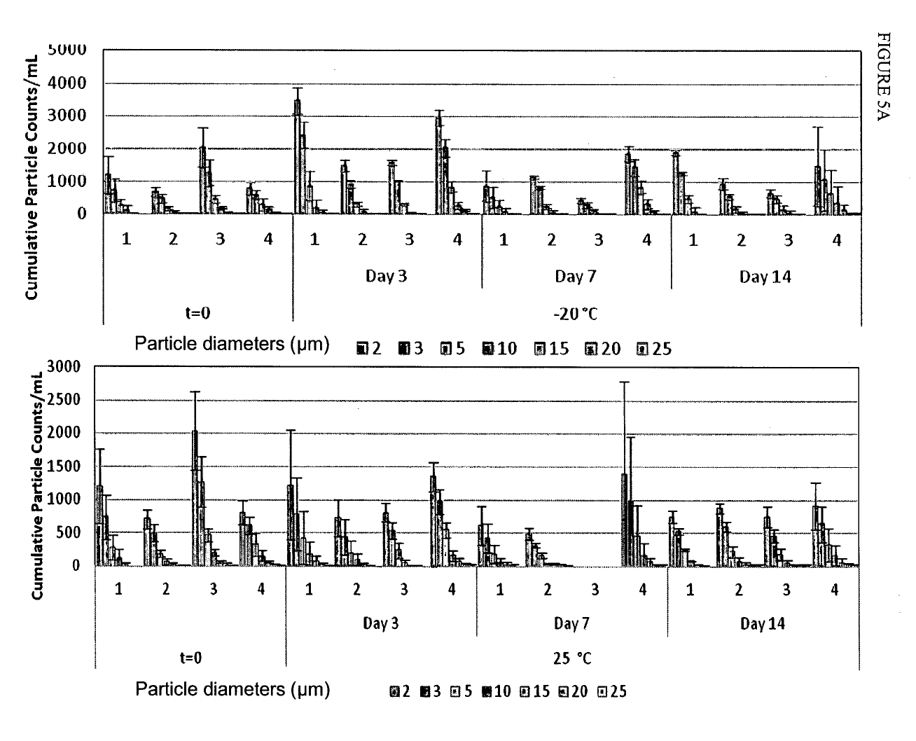

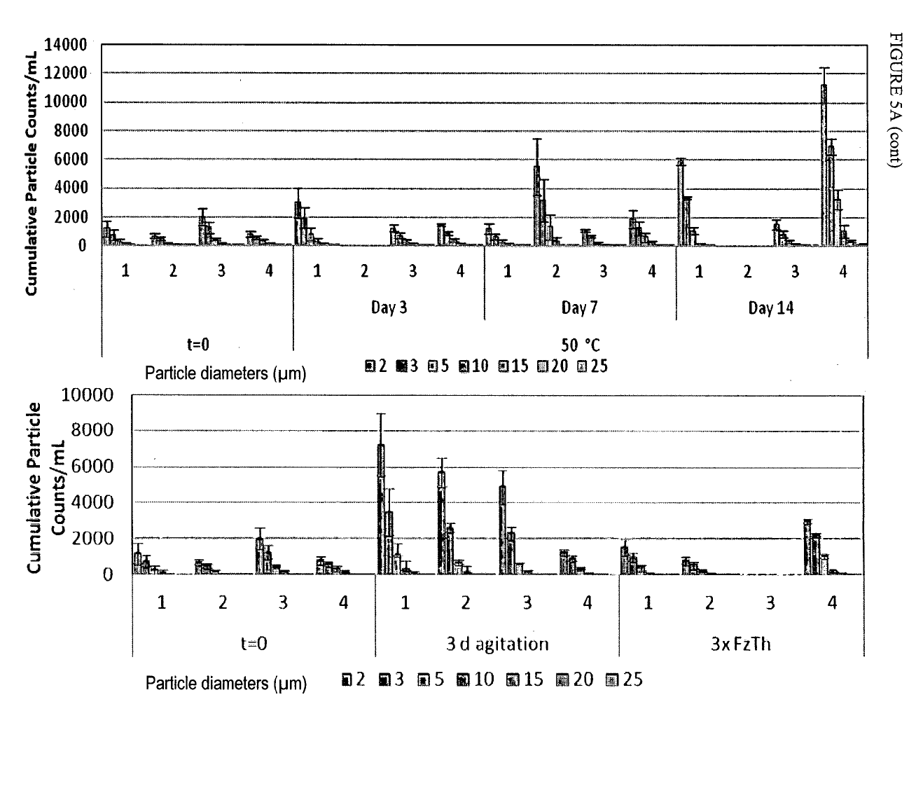

FIG. 4A shows turbidity measures (Absorbance at 330 nm) at all times (from 0 to 14 days) and conditions (−20° C., 25° C., 50° C., 3 times freezing/thawing (3×FzTh) and 3 days in agitation). According to the results, significant increases in turbidity were detected at the 50° C. condition, with F3 presenting the lowest increase over time. No significant changes were observed in any formulation at −20° C., 25° C., freeze-thaw or agitation.

FIG. 4B(1) shows turbidity measures for formulation F3 (Absorbance at 330 nm) at times t=0, 1 and 3 months and conditions (−20° C., 2-8° C., 25° C., 1 time freezing/thawing (1× and 2×FzTh (−20/25° C.)). As can be seen in FIG. 4B(1), slight increase in turbidity was observed for the samples subjected to 3 month storage at 25° C. No changes were observed after 3 months for samples stored at −20° C., 2-8° C. and subjected to 2 freeze-thaw cycles. Data for constructing said FIG. 4B(1) is provided in the following table:

| | | | Time Point | A330, |
|---|---|---|---|---|
| Formulation | Condition | | (months) | AU |
| F3 | t = 0 Control | | 0 | 0.202 |
| | −20° | C. | 1 | 0.200 |
| | | | 3 | 0.202 |
| | | | 6 | 0.213 |
| | 25° | C. | 1 | 0.212 |
| | | | 3 | 0.220 |
| | | | 6 | 0.227 |
| | 2-8° | C. | 1 | 0.211 |

| | | |
|---|---|---|
| | 3 | 0.199 |
| | 6 | 0.197 |
| Fz Th (−20° C./25° C.) | 1x | 0.217 |
| | 2x | 0.208 |
| | 4x | 0.200 |

The following table summarizes the data obtained for formulations F1, F5, F6, F7, F8, F9 at t=0 and t=3 months and after 1, 2, and 4 cycles of freeze-thaw at −20° C./25° C. and Innovator (control) at t=0 and 25° C. Formulations F1, F5, and F8 presented no major changes in turbidity. F6 presented the highest variation in turbidity when stored at 25° C.

| | | | Time Point | A330, |
|---|---|---|---|---|
| Formulation | Condition | | (months) | AU |
| F1 | t = 0 Control | | 0 | 0.191 |
| | −20° | C. | 1 | 0.198 |
| | | | 3 | 0.195 |
| | 25° | C. | 1 | 0.207 |
| | | | 3 | 0.193 |
| | 2-8° | C. | 1 | 0.215 |
| | | | 3 | 0.199 |
| | Fz Th (−20° C./25° C.) | | 1x | 0.191 |
| | | | 2x | 0.219 |
| | | | 4x | 0.180 |
| F5 | t = 0 Control | | 0 | 0.200 |
| | −20° | C. | 1 | 0.228 |
| | | | 3 | 0.203 |
| | 25° | C. | 1 | 0.207 |
| | | | 3 | 0.220 |
| | 2-8° | C. | 1 | 0.215 |
| | | | 3 | 0.185 |
| | Fz Th (−20° C./25° C.) | | 1x | 0.196 |
| | | | 2x | 0.206 |
| | | | 4x | 0.209 |
| F6 | t = 0 Control | | 0 | 0.193 |
| | −20° | C. | 1 | 0.217 |
| | | | 3 | 0.208 |
| | 25° | C. | 1 | 0.446 |
| | | | 3 | 0.371 |
| | 2-8° | C. | 1 | 0.194 |
| | | | 3 | 0.198 |
| | Fz Th (−20° C./25° C.) | | 1x | 0.195 |
| | | | 2x | 0.208 |
| | | | 4x | 0.183 |
| F8 | t = 0 Control | | 0 | 0.192 |
| | −20° | C. | 1 | 0.206 |
| | | | 3 | 0.185 |
| | 25° | C. | 1 | 0.205 |
| | | | 3 | 0.203 |
| | 2-8° | C. | 1 | 0.191 |
| | | | 3 | 0.195 |
| | Fz Th (−20° C./25° C.) | | 1x | 0.197 |
| | | | 2x | 0.208 |
| | | | 4x | 0.188 |
| Innovator | t = 0 Control | | 0 | 0.182 |
| | 25° | C. | 3 | 0.180 |

As stated above, no significant further increase in turbidity was observed for formulations F5, F8 or F1 after 1 or 3 months at all conditions and as compared to t=0 (FIG. 4B(2)).

### HIAC (Liquid Particle Counter)

### Method:

A HIAC 9703 Liquid Particle Counting System was used for the experiments. The HIAC consists of a sampler, particle counter and Royco sensor. The Royco sensor is capable of sizing and counting particles between 2 μm to 100 μm. The instrument can count particles≦10,000 counts/mL.
Sample volume (mL): 0.2
Flow rate mL/min: 10
Number of runs (per sample): 4 (first run is discarded)

### Procedure:

Initially samples were analysed without dilution, but due to the sample's high viscosity it was determined that they needed to be diluted to obtain a more accurate result.
Samples were brought to room temperature for 1 hr.
Samples were diluted 1:3 in the appropriate formulation buffer, degassed (1.5 hrs) and carefully mixed prior to measurement.
Standards-Duke Scientific Count Cal:System suitability checks are performed with the EZY-Cal 5 m and 15 μm particle size control standards. The control standards are analyzed at the beginning to verify resolution of the sensor.

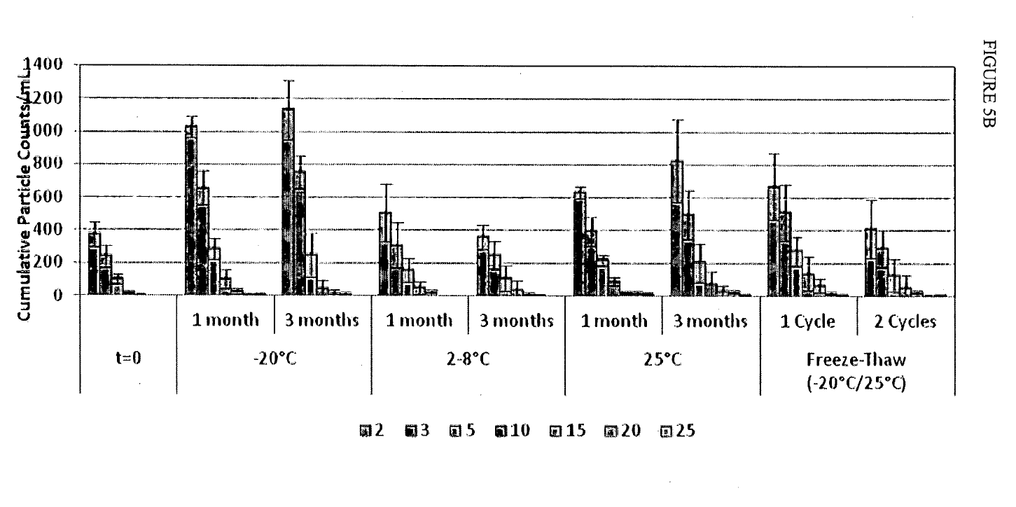

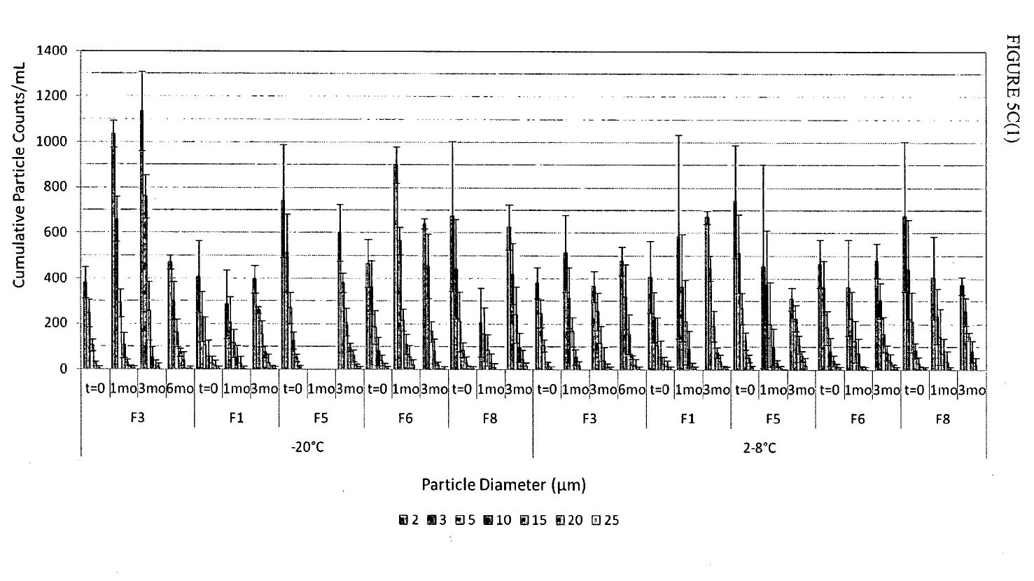

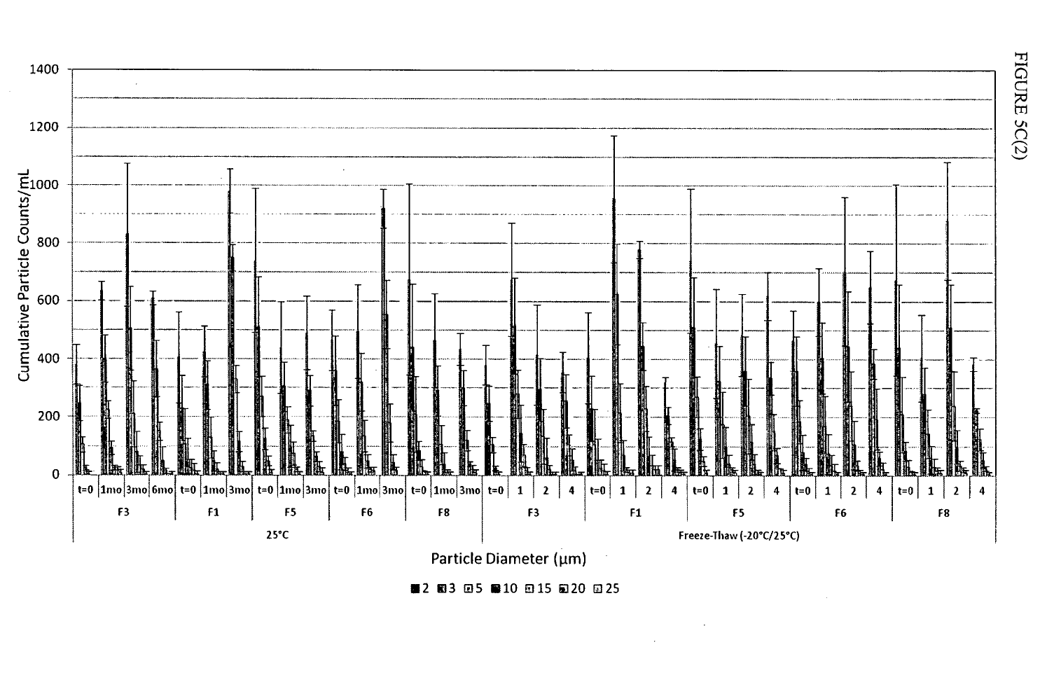

FIG. 5A shows sub-visible particle analysis by HIAC measured at all conditions: −20° C., 25° C., 50° C., 3 times freezing/thawing (3×FzTh) and 3 days in agitation using the Standards-Duke Scientific Count Cal.

As can be seen in FIG. 5A, significant increases in subvisible particle counts were measured at the 50° C. condition for F1, F2 and F4, with F2 showing the highest increase from as early as 7 days.

No significant changes were observed for any formulation at −20° C., 25° C., 3×FzTh or after 3 d RT agitation. F3 formulation presented no change in subvisible particle as compared to t=0 control after storage under all conditions and time points.

Figure SB shows sub-visible particle analysis by HIAC for formulation F3 measured at t=0, 1 and 3 months and at −20° C., 2-8° C., 25° C., 1 and 2 times freezing/thawing (1× and 2×FzTh at −20° C./25° C.) using the Standards-Duke Scientific Count Cal. As can be seen in FIG. 5B, slight further increase in sub-visible particle counts for the 25° C. condition at 3 months is observed. The −20° C. condition presents the greatest increase in sub-visible particles by 3 months. No changes are observed from t=0 for the 2-8° C. timepoint after 3 months or after 2 cycles of freeze-thaw. A slight further increase is observed from 1 month in sub-visible particle counts at the −20° C. condition.

Data for constructing said figure SB is provided in the following table:

| | | Particle diameters (μm) | | | | | | |
|---|---|---|---|---|---|---|---|---|
| | | Cumulative Particle Counts/mL | | | | | | |
| | | Diameter (μm) | | | | | | |
| Condition | Time Point | 2 | 3 | 5 | 10 | 15 | 20 | 25 |
| t = 0 | | 380 ± 69 | 245 ± 61 | 105 ± 26 | 25 ± 9 | 5 ± 9 | 0 ± 0 | 0 ± 0 |
| −20° C. | 1 month | 1035 ± 60 | 660 ± 98 | 290 ± 61 | 105 ± 54 | 30 ± 15 | 10 ± 9 | 5 ± 9 |
| | 3 months | 1135 ± 174 | 760 ± 95 | 255 ± 130 | 50 ± 48 | 15 ± 26 | 10 ± 17 | 0 ± 0 |
| 2-8° C. | 1 month | 510 ± 169 | 315 ± 133 | 165 ± 65 | 55 ± 35 | 20 ± 17 | 0 ± 0 | 0 ± 0 |
| | 3 months | 365 ± 69 | 255 ± 84 | 115 ± 75 | 40 ± 57 | 10 ± 17 | 5 ± 9 | 0 ± 0 |
| 25° C. | 1 month | 635 ± 31 | 400 ± 83 | 225 ± 30 | 95 ± 23 | 25 ± 9 | 15 ± 15 | 10 ± 9 |
| | 3 months | 830 ± 248 | 505 ± 144 | 210 ± 113 | 80 ± 71 | 35 ± 31 | 20 ± 17 | 5 ± 9 |
| Freeze-Thaw | 1 Cycle | 675 ± 196 | 515 ± 166 | 280 ± 83 | 145 ± 98 | 70 ± 38 | 15 ± 15 | 5 ± 9 |
| (−20° C./25° C.) | 2 Cycles | 415 ± 173 | 295 ± 109 | 135 ± 94 | 60 ± 69 | 20 ± 17 | 5 ± 9 | 5 ± 9 |

FIG. 5C(1 and 2) shows sub-visible particle analysis by HIAC for formulations F1, F3, F5, F6 and F8 measured at t=0, 1 and 3 months and at −20° C., 2-8° C. (FIG. 5C(1)), 25° C., 1, 2, 3 and 4 times freezing/thawing (1×, 2×, 3× and 4×FzTh at −20° C./25° C.) (FIG. 5C(2)) using the Standards-Duke Scientific Count Cal.

Data for constructing said FIG. 5C(1) is provided in the following table.

| | | | Diameter | | | | | | |
|---|---|---|---|---|---|---|---|---|---|
| Condition | Formulation | Time Point | 2 | 3 | 5 | 10 | 15 | 20 | 25 |
| −20° C. | F3 | t = 0 | 380 ± 69 | 245 ± 61 | 105 ± 26 | 25 ± 9 | 5 ± 9 | 0 ± 0 | 0 ± 0 |
| | | 1 mo | 1035 ± 60 | 660 ± 98 | 290 ± 61 | 105 ± 54 | 30 ± 15 | 10 ± 9 | 5 ± 9 |
| | | 3 mo | 1135 ± 174 | 760 ± 95 | 255 ± 130 | 50 ± 48 | 15 ± 26 | 10 ± 17 | 0 ± 0 |
| | | 6 mo | 470 ± 31 | 300 ± 85 | 160 ± 61 | 75 ± 17 | 40 ± 35 | 5 ± 0 | 5 ± 9 |
| | F1 | t = 0 | 405 ± 158 | 230 ± 111 | 105 ± 123 | 55 ± 71 | 25 ± 31 | 15 ± 26 | 5 ± 9 |
| | | 1 mo | 285 ± 152 | 205 ± 115 | 115 ± 61 | 50 ± 53 | 25 ± 31 | 5 ± 9 | 0 ± 0 |
| | | 3 mo | 395 ± 60 | 260 ± 15 | 155 ± 57 | 75 ± 26 | 30 ± 35 | 10 ± 9 | 5 ± 9 |
| | F5 | t = 0 | 740 ± 250 | 510 ± 173 | 270 ± 69 | 125 ± 38 | 50 ± 17 | 10 ± 9 | 0 ± 0 |
| | | 1 mo | | | | | | | |
| | | 3 mo | 600 ± 125 | 380 ± 43 | 205 ± 61 | 95 ± 17 | 60 ± 26 | 15 ± 9 | 5 ± 9 |
| | F6 | t = 0 | 465 ± 105 | 360 ± 119 | 185 ± 74 | 80 ± 61 | 40 ± 23 | 10 ± 17 | 5 ± 9 |
| | | 1 mo | 900 ± 79 | 565 ± 61 | 215 ± 48 | 110 ± 43 | 55 ± 48 | 20 ± 23 | 0 ± 0 |
| | | 3 mo | 640 ± 23 | 455 ± 142 | 165 ± 46 | 80 ± 52 | 20 ± 15 | 5 ± 0 | 5 ± 9 |
| | F8 | t = 0 | 675 ± 332 | 440 ± 219 | 210 ± 130 | 85 ± 31 | 30 ± 26 | 15 ± 0 | 5 ± 9 |
| | | 1 mo | 205 ± 150 | 155 ± 117 | 85 ± 68 | 30 ± 40 | 10 ± 17 | 0 ± 0 | 0 ± 0 |
| | | 3 mo | 625 ± 100 | 420 ± 133 | 240 ± 122 | 95 ± 62 | 45 ± 40 | 15 ± 15 | 0 ± 0 |
| 2-8° C. | F3 | t = 0 | 380 ± 69 | 245 ± 61 | 105 ± 26 | 25 ± 9 | 5 ± 9 | 0 ± 0 | 0 ± 0 |
| | | 1 mo | 510 ± 169 | 315 ± 133 | 165 ± 65 | 55 ± 35 | 20 ± 17 | 0 ± 0 | 0 ± 0 |
| | | 3 mo | 365 ± 69 | 255 ± 84 | 115 ± 75 | 40 ± 57 | 10 ± 17 | 5 ± 9 | 0 ± 0 |
| | | 6 mo | 475 ± 62 | 320 ± 143 | 155 ± 85 | 55 ± 9 | 20 ± 26 | 5 ± 9 | 0 ± 0 |
| | F1 | t = 0 | 405 ± 158 | 230 ± 111 | 105 ± 123 | 55 ± 71 | 25 ± 31 | 15 ± 26 | 5 ± 9 |
| | | 1 mo | 585 ± 448 | 360 ± 236 | 210 ± 184 | 90 ± 79 | 15 ± 15 | 5 ± 9 | 0 ± 0 |
| | | 3 mo | 670 ± 30 | 445 ± 54 | 190 ± 68 | 75 ± 23 | 35 ± 31 | 10 ± 9 | 5 ± 9 |
| | F5 | t = 0 | 740 ± 250 | 510 ± 173 | 270 ± 69 | 125 ± 38 | 50 ± 17 | 10 ± 9 | 0 ± 0 |
| | | 1 mo | 455 ± 448 | 375 ± 236 | 200 ± 184 | 100 ± 79 | 30 ± 15 | 10 ± 9 | 5 ± 0 |
| | | 3 mo | 310 ± 48 | 225 ± 57 | 110 ± 38 | 60 ± 23 | 20 ± 35 | 0 ± 0 | 0 ± 0 |
| | F6 | t = 0 | 465 ± 105 | 360 ± 119 | 185 ± 74 | 80 ± 61 | 40 ± 23 | 10 ± 17 | 5 ± 9 |
| | | 1 mo | 360 ± 212 | 225 ± 120 | 125 ± 90 | 70 ± 68 | 10 ± 9 | 5 ± 9 | 0 ± 0 |
| | | 3 mo | 480 ± 75 | 305 ± 78 | 155 ± 77 | 75 ± 31 | 35 ± 35 | 15 ± 9 | 5 ± 9 |
| | F8 | t = 0 | 675 ± 332 | 440 ± 219 | 210 ± 130 | 85 ± 31 | 30 ± 26 | 15 ± 0 | 5 ± 9 |
| | | 1 mo | 405 ± 182 | 235 ± 121 | 145 ± 121 | 70 ± 68 | 35 ± 48 | 5 ± 9 | 0 ± 0 |
| | | 3 mo | 370 ± 38 | 255 ± 61 | 145 ± 17 | 80 ± 45 | 20 ± 35 | 0 ± 0 | 0 ± 0 |

FIG. 5C(2) shows sub-visible particle analysis by HIAC measured for formulations F1, F5, F6, and F8 at t=0, t=1 month and t=3 months, and 1, 2 and 4 times freezing/thawing (1×, 2× and 4×FzTh) at −20° C./25° C. using the Standards-Duke Scientific Count Cal.

Data for constructing FIG. 5C(2) is provided in the following table.

| | | | Diameter | | | | | | |
|---|---|---|---|---|---|---|---|---|---|
| Condition | Formulation | Time Point | 2 | 3 | 5 | 10 | 15 | 20 | 25 |
| 25° C. | F3 | t = 0 | 380 ± 69 | 245 ± 61 | 105 ± 26 | 25 ± 9 | 5 ± 9 | 0 ± 0 | 0 ± 0 |
| | | 1 mo | 635 ± 31 | 400 ± 83 | 225 ± 30 | 95 ± 23 | 25 ± 9 | 15 ± 15 | 10 ± 9 |
| | | 3 mo | 830 ± 248 | 505 ± 144 | 210 ± 113 | 80 ± 71 | 35 ± 31 | 20 ± 17 | 5 ± 9 |
| | | 6 mo | 610 ± 23 | 365 ± 98 | 150 ± 31 | 50 ± 48 | 15 ± 9 | 5 ± 0 | 5 ± 9 |
| | F1 | t = 0 | 405 ± 158 | 230 ± 111 | 105 ± 123 | 55 ± 71 | 25 ± 31 | 15 ± 26 | 5 ± 9 |
| | | 1 mo | 425 ± 88 | 310 ± 85 | 130 ± 71 | 50 ± 35 | 20 ± 23 | 5 ± 9 | 5 ± 9 |
| | | 3 mo | 980 ± 77 | 750 ± 45 | 330 ± 48 | 115 ± 35 | 30 ± 17 | 5 ± 0 | 5 ± 9 |
| | F5 | t = 0 | 740 ± 250 | 510 ± 173 | 270 ± 69 | 125 ± 38 | 50 ± 17 | 10 ± 9 | 0 ± 0 |
| | | 1 mo | 440 ± 159 | 305 ± 85 | 190 ± 46 | 100 ± 71 | 75 ± 40 | 20 ± 9 | 5 ± 9 |
| | | 3 mo | 490 ± 128 | 290 ± 53 | 135 ± 17 | 65 ± 17 | 30 ± 17 | 10 ± 17 | 0 ± 0 |
| | F6 | t = 0 | 465 ± 105 | 360 ± 119 | 185 ± 74 | 80 ± 61 | 40 ± 23 | 10 ± 17 | 5 ± 9 |
| | | 1 mo | 495 ± 162 | 320 ± 100 | 135 ± 54 | 50 ± 23 | 20 ± 9 | 15 ± 15 | 0 ± 0 |
| | | 3 mo | 920 ± 68 | 555 ± 117 | 180 ± 65 | 45 ± 26 | 15 ± 15 | 0 ± 0 | 0 ± 0 |
| | F8 | t = 0 | 675 ± 332 | 440 ± 219 | 210 ± 130 | 85 ± 31 | 30 ± 26 | 15 ± 0 | 5 ± 9 |
| | | 1 mo | 465 ± 162 | 290 ± 87 | 105 ± 65 | 40 ± 38 | 10 ± 9 | 5 ± 9 | 0 ± 0 |
| | | 3 mo | 435 ± 54 | 300 ± 60 | 120 ± 35 | 40 ± 9 | 20 ± 17 | 10 ± 9 | 0 ± 0 |
| Freeze-Thaw | F3 | t = 0 | 380 ± 69 | 245 ± 61 | 105 ± 26 | 25 ± 9 | 5 ± 9 | 0 ± 0 | 0 ± 0 |
| (−20° C./25° C.) | | 1 | 675 ± 196 | 515 ± 166 | 280 ± 83 | 145 ± 98 | 70 ± 38 | 15 ± 15 | 5 ± 9 |
| | | 2 | 415 ± 173 | 295 ± 109 | 135 ± 94 | 60 ± 69 | 20 ± 17 | 5 ± 9 | 5 ± 9 |
| | | 4 | 355 ± 69 | 255 ± 91 | 105 ± 35 | 55 ± 38 | 15 ± 17 | 5 ± 0 | 5 ± 9 |
| | F1 | t = 0 | 405 ± 158 | 230 ± 111 | 105 ± 123 | 55 ± 71 | 25 ± 31 | 15 ± 26 | 5 ± 9 |
| | | 1 | 955 ± 220 | 625 ± 174 | 215 ± 100 | 70 ± 53 | 20 ± 9 | 10 ± 9 | 10 ± 9 |
| | | 2 | 780 ± 30 | 445 ± 83 | 230 ± 77 | 65 ± 68 | 35 ± 38 | 20 ± 17 | 20 ± 17 |
| | | 4 | 320 ± 17 | 205 ± 30 | 115 ± 15 | 55 ± 38 | 20 ± 9 | 10 ± 9 | 5 ± 9 |
| | F5 | t = 0 | 740 ± 250 | 510 ± 173 | 270 ± 69 | 125 ± 38 | 50 ± 17 | 10 ± 9 | 0 ± 0 |
| | | 1 | 455 ± 189 | 325 ± 122 | 175 ± 113 | 100 ± 68 | 40 ± 31 | 20 ± 9 | 10 ± 9 |
| | | 2 | 485 ± 143 | 360 ± 120 | 205 ± 128 | 115 ± 61 | 40 ± 35 | 10 ± 9 | 5 ± 9 |
| | | 4 | 620 ± 84 | 335 ± 57 | 150 ± 62 | 70 ± 26 | 25 ± 0 | 10 ± 9 | 0 ± 0 |
| | F6 | t = 0 | 465 ± 105 | 360 ± 119 | 185 ± 74 | 80 ± 61 | 40 ± 23 | 10 ± 17 | 5 ± 9 |
| | | 1 | 600 ± 117 | 405 ± 123 | 170 ± 102 | 75 ± 69 | 35 ± 38 | 15 ± 26 | 5 ± 9 |
| | | 2 | 705 ± 256 | 445 ± 190 | 240 ± 119 | 105 ± 84 | 35 ± 17 | 10 ± 9 | 5 ± 9 |
| | | 4 | 650 ± 125 | 385 ± 53 | 195 ± 105 | 60 ± 23 | 20 ± 26 | 5 ± 9 | 0 ± 0 |
| | F8 | t = 0 | 675 ± 332 | 440 ± 219 | 210 ± 130 | 85 ± 31 | 30 ± 26 | 15 ± 0 | 5 ± 9 |
| | | 1 | 405 ± 150 | 280 ± 92 | 145 ± 83 | 55 ± 48 | 30 ± 30 | 15 ± 15 | 10 ± 9 |
| | | 2 | 880 ± 204 | 510 ± 150 | 240 ± 119 | 100 ± 57 | 35 ± 17 | 20 ± 9 | 10 ± 9 |
| | | 4 | 385 ± 23 | 225 ± 9 | 125 ± 38 | 55 ± 26 | 25 ± 9 | 5 ± 9 | 0 ± 0 |

As can be seen in FIG. 5C, no significant changes in sub-visible particle counts were observed for F1, F3, F5, F6 and F8 from t=0 for the 2-8° C. time point after 3 months. In addition, F1 and F6 performed similarly at 25° C., increasing in sub-visible particles over time up to 3 months. No significant changes in F8 over time at 25° C., showing the stability of this formulation.

No significant changes in sub-visible particle counts were observed for the control sample (Innovator product) after 3 months at 25° C. The Innovator product presented the highest particle count over time and as compared to F1, F3, F5, F6 and F8 (see table below).

| | | Diameter | | | | | | |
|---|---|---|---|---|---|---|---|---|
| | Time point | 2 | 3 | 5 | 10 | 15 | 20 | 25 |
| Innovator | t = 0 | 42495 ± 1233 | 31200 ± 1280 | 13590 ± 1130 | 3270 ± 559 | 1095 ± 104 | 405 ± 156 | 150 ± 69 |
| | 3 months | 27917 ± 447 | 18308 ± 1455 | 6858 ± 486 | 1150 ± 29 | 358 ± 52 | 117 ± 14 | 33 ± 14 |

### SDS-PAGE

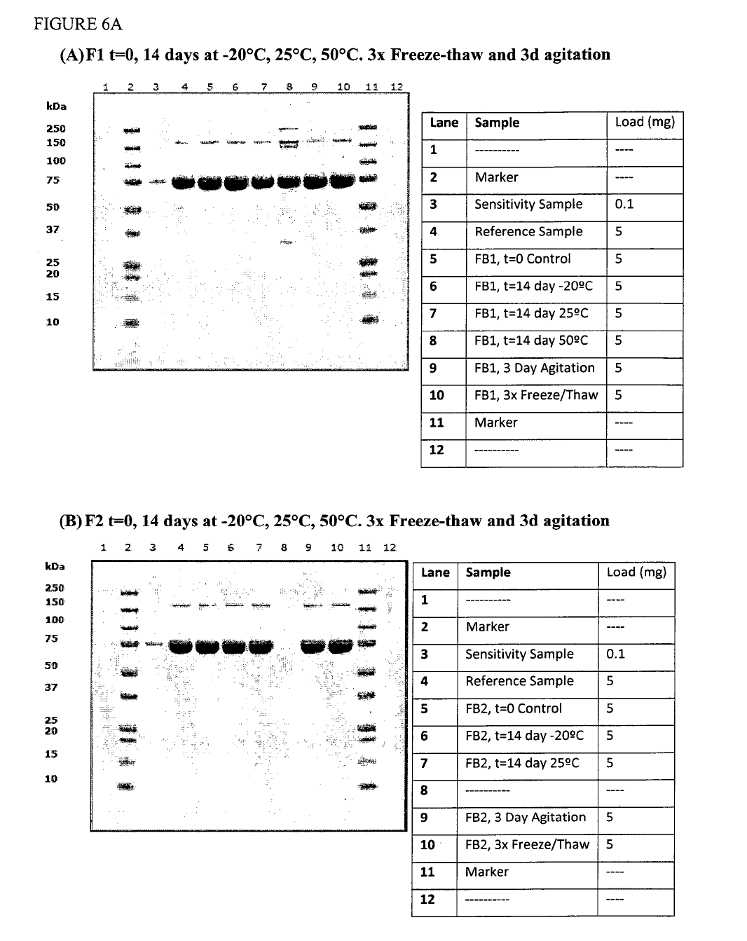

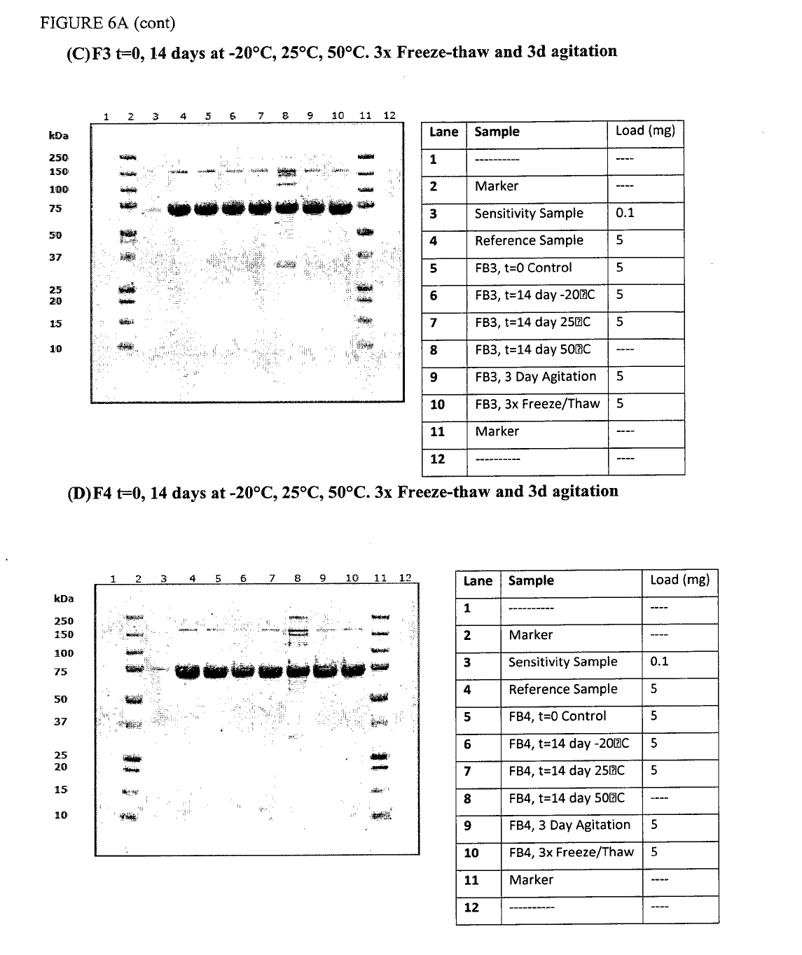

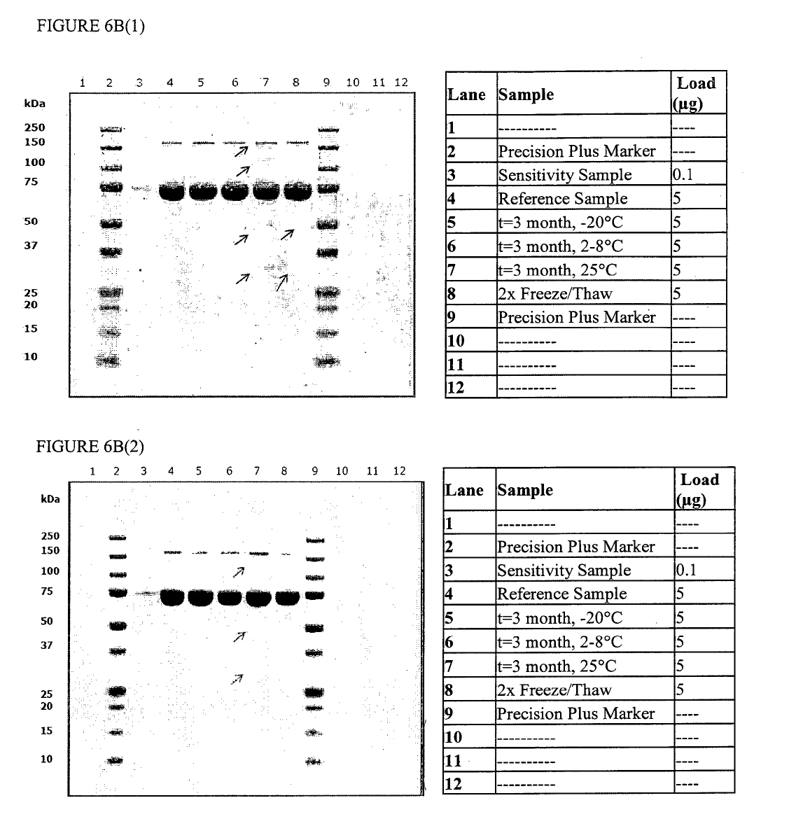

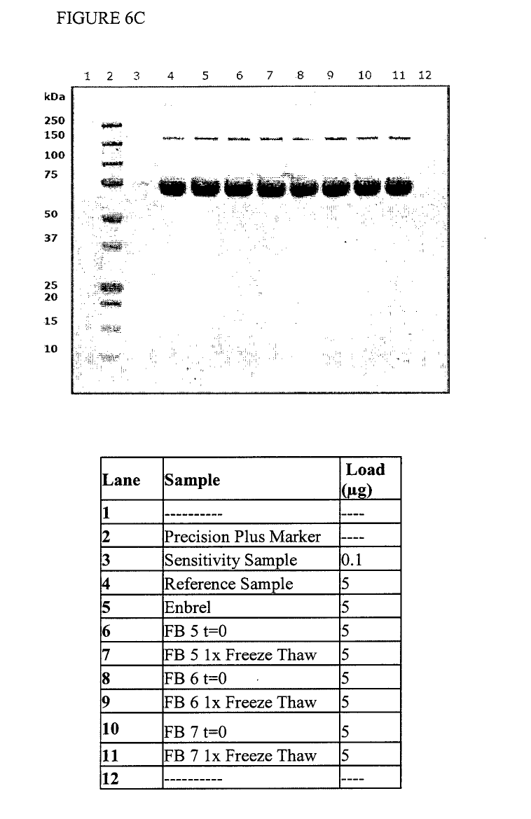

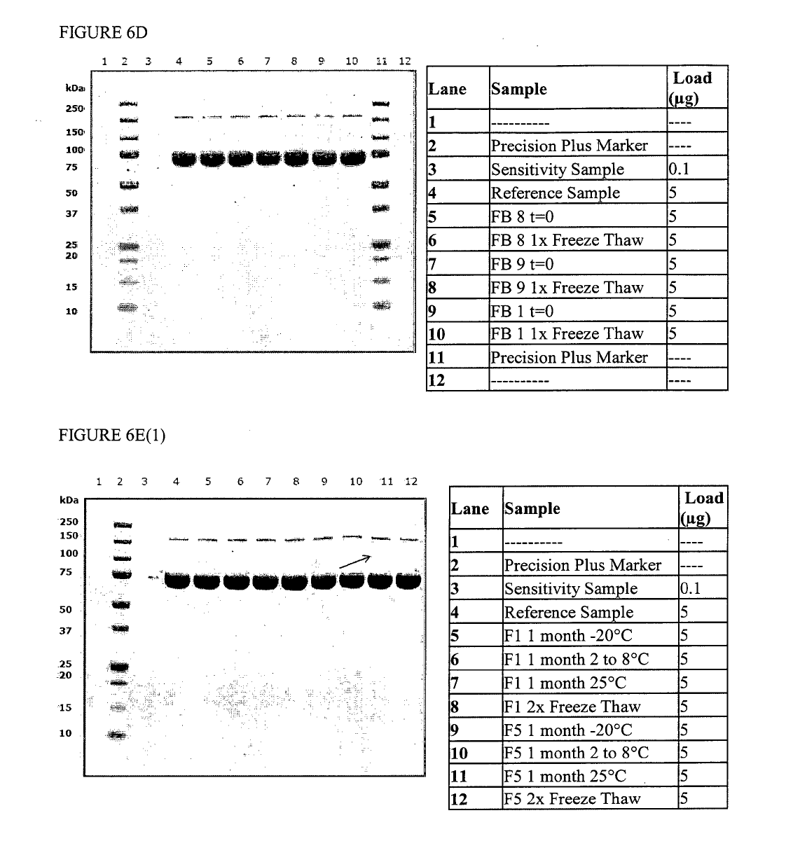

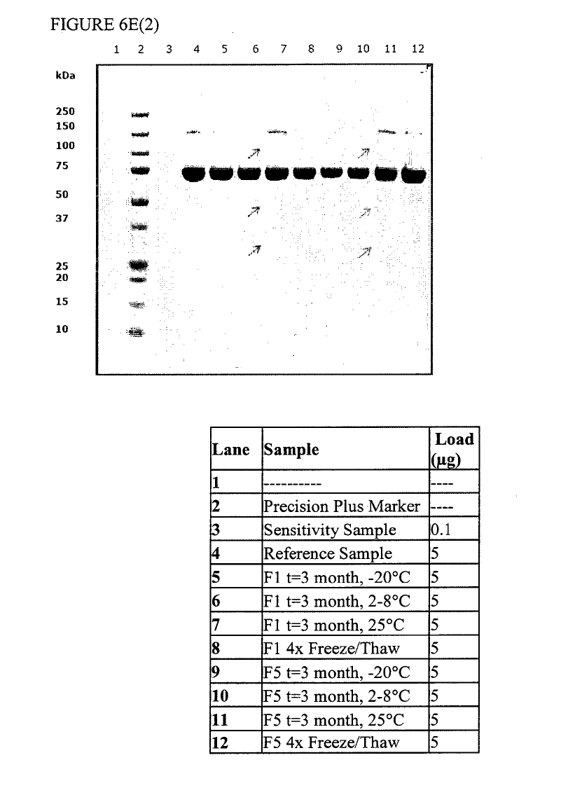

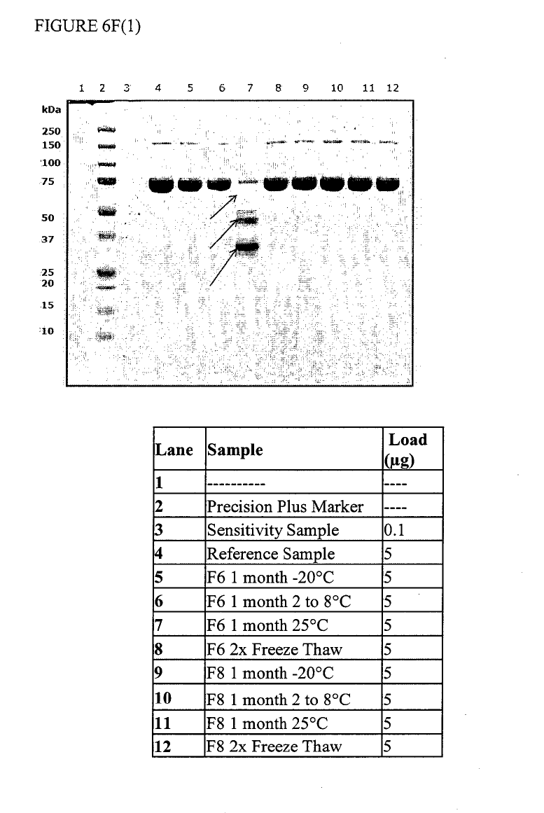

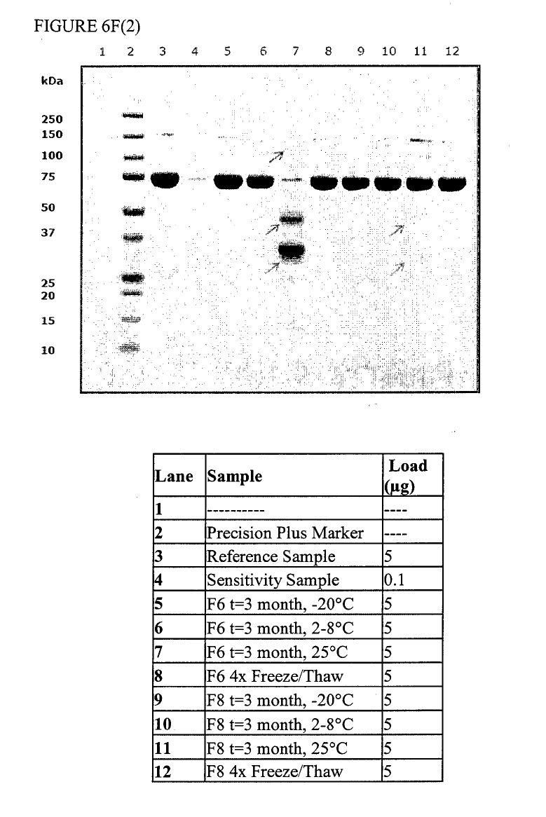

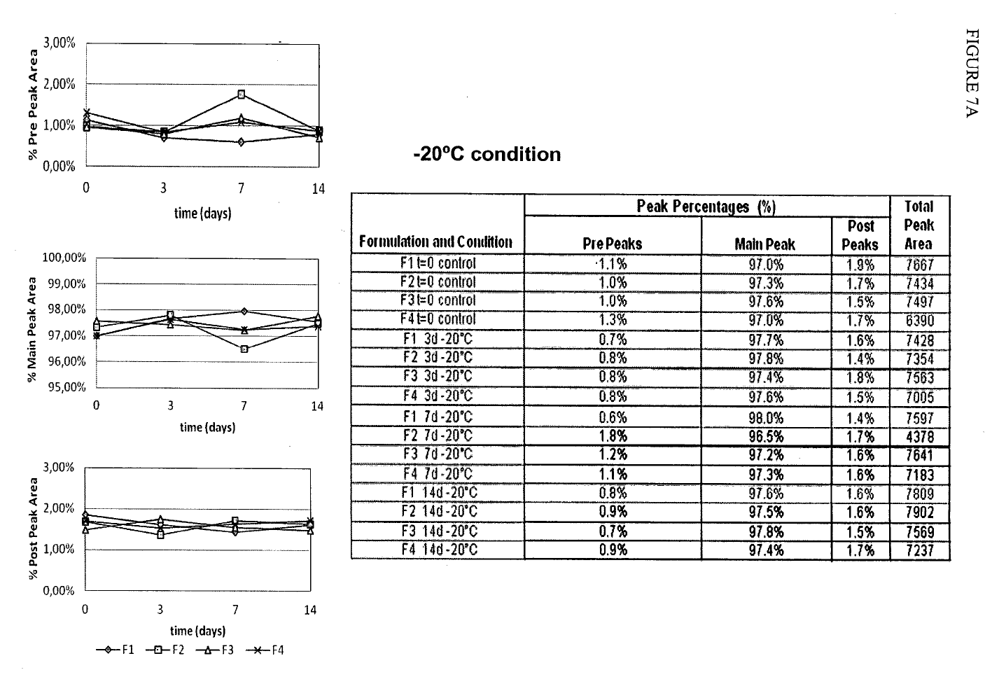

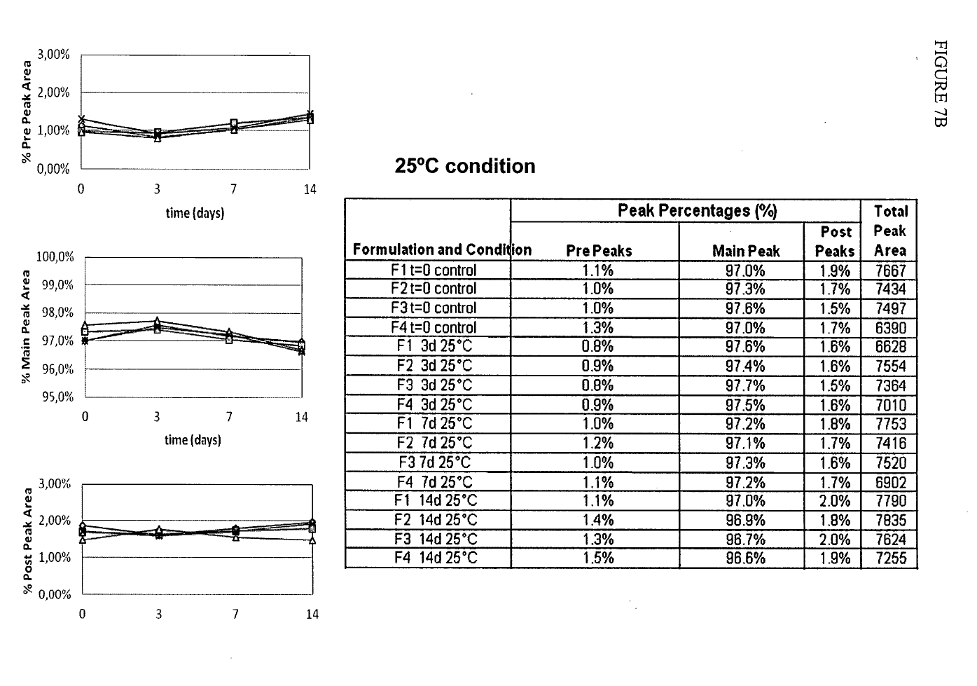

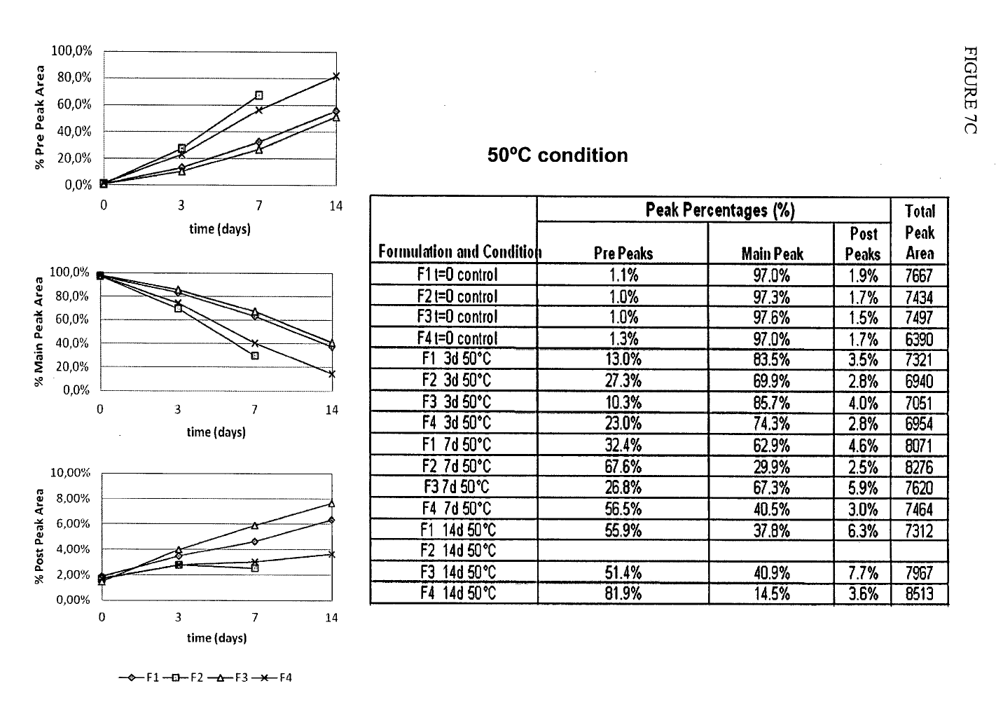

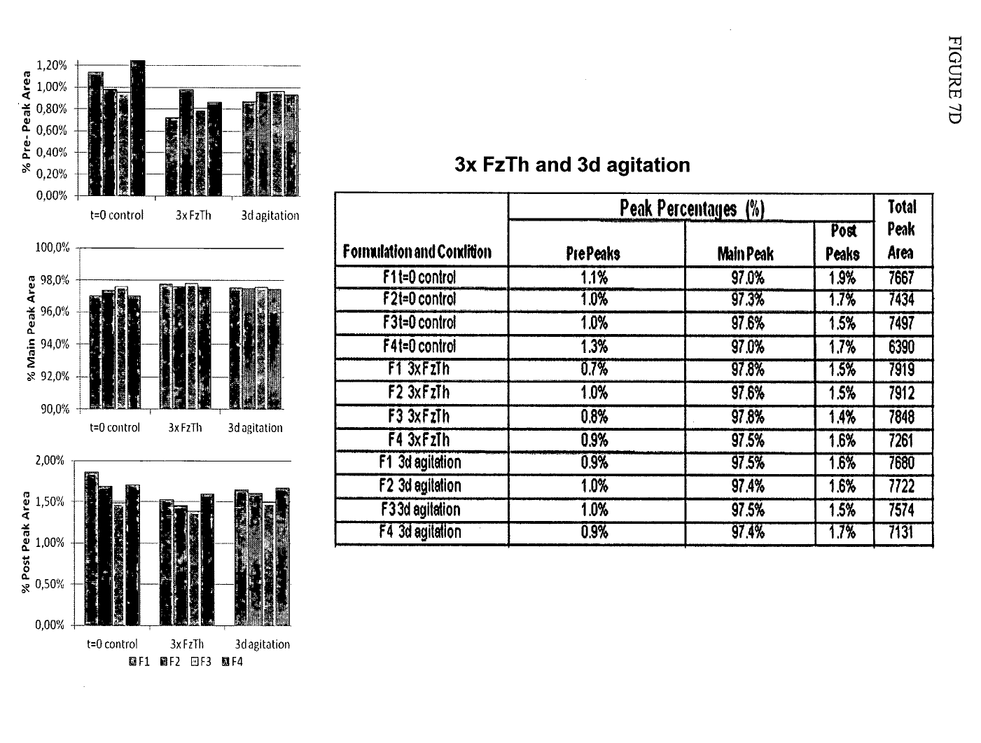

FIG. 6A shows SDS-PAGE gels stained with Coomassie incubated at all conditions: −20° C., 25° C., 50° C., 3 times freezing/thawing and 3 days in agitation at times 0 and 14 days. In (A), F1 sample, in (B) F2 sample, in (C) F3 sample and in (D) F4 sample.

Significant changes observed in all formulations for the 50° C. condition at all timepoints, with day 14 samples showing likely covalently-modified high molecular weight (HMW) species as evidenced by additional HMW bands present (>˜250 kDa) and low molecular weight (LMW) breakdown species (<50 kDa), which were present from as early as 3 days at 50° C. for all formulations.

No changes were observed in any formulation for all other conditions and time points and as compared to the reference standard.

FIG. 6B(1) shows SDS-PAGE gels stained with Coomassie for formulation F3 at t=3 months incubated at all conditions: −20° C., 2-8° C., 25° C., 2 times freezing/thawing at −20° C./25° C.

Changes were observed after 3 months at 25° C., with appearance of extra bands at ˜100 kDa and ˜140 kDa and an increase in intensity of LMW (low molecular weight) breakdown bands at ˜50 kDa and ˜30 kDa.

Changes were observed after 2 cycles of freeze-thaw (−20° C./25° C.) with darkening of ˜30 kDa and ˜50 kDa bands.

FIG. 6B(2) shows SDS-PAGE gels stained with Coomassie for formulation F3 at t=6 months incubated at all conditions: −20° C., 2-8° C., 25° C., 4 times freezing/thawing at −20° C./25° C.

Changes are observed for F3 after 6 months at 25° C., with the appearance of an extra band at ˜100 kDa and an increase in intensity of LMW breakdown bands at ˜50 kDa and ˜30 kDa.

FIG. 6C shows SDS-PAGE gels stained with Coomassie for formulations F5, F6 and F7 and Innovator (control) at t=0 and after 1 time freezing/thawing at −20° C./25° C. condition.

Formulations F5, F6, F7 and Innovator (control) at t=0 are comparable to the reference standard.

Formulations F5, F6, F7 after 1 cycle freeze-thaw at −20° C./25° C. are comparable to the reference standard.

FIG. 6D shows SDS-PAGE gels stained with Coomassie for formulations F8, F9 and F1 and Innovator (control) at t=0 and after 1 time freezing/thawing at −20° C./25° C. condition.

Formulations F8, F9, F1 at t=0 and after 1 cycle freeze-thaw at −20° C./25° C. are comparable to the reference standard.

FIG. 6E(1) shows SDS-PAGE gels stained with Coomassie for formulations F1 and F5 at t=1 month at −20° C., 2-8° C. and 25° C. and after 2 cycles freezing/thawing at −20° C./25° C. condition.

Formulations F1 and F5 at all conditions at the 1 month timepoint are comparable to the reference standard.

Slight evidence of additional ˜100 kDa band for formulation F5 is shown after 1 month at 25° C.

FIG. 6E(2) shows SDS-PAGE gels stained with Coomassie for formulations F1 and F5 at t=3 month at −20° C., 2-8° C. and 25° C. and after 4 cycles freezing/thawing at −20° C./25° C. condition.

Slight evidence of the appearance of very faint bands at ˜100 kDa, ˜50 kDa and ˜30 kD for F5 after 3 months at 25° C. and as compared to F1 after 3 months at 25° C., which also demonstrates these additional bands.

FIG. 6F(1) shows SDS-PAGE gels stained with Coomassie for formulations F6 and F8 at t=1 month at −20° C., 2-8° C. and 25° C. and after 2 cycles freezing/thawing at −20° C./25° C. condition.

Formulations F6 and F8 at −20° C. and 2-8° C. after 1 month, including the 2 cycles freezing/thawing at −20° C./25° C., are shown to be comparable to the reference standard.

Formulation F6 after 1 month at 25° C. demonstrates almost complete loss of the main band with several additional low molecular weight breakdown bands evident.

FIG. 6F(2) shows SDS-PAGE gels stained with Coomassie for formulations F6 and F8 at t=3 month at −20° C., 2-8° C. and 25° C. and after 2 cycles freezing/thawing at −20° C./25° C. condition.

Significant changes are observed for F6 after 3 months at 25° C., with disappearance of the 150 kD band and appearance of several LMW breakdown bands. Only slight evidence of the appearance of very faint bands at ˜50 kDa and ˜30 kD is shown for both F6 and F8.

### SE HPLC (Size Exclusion HPLC)

### Conditions:

Column: TSKGel SuperSW3000 4.6×300 mm, 4 μm (Tosoh, 18675) CV=2.5 mL
Column Temp: 25° C.
Mobile Phase: 0.2 M Phosphate Buffer, pH 6.8
Flow Rate: 0.35 mL/min
Runtime: 20 min
Sample Load: 37.6 μg
Auto Sampler Temperature: 4° C.

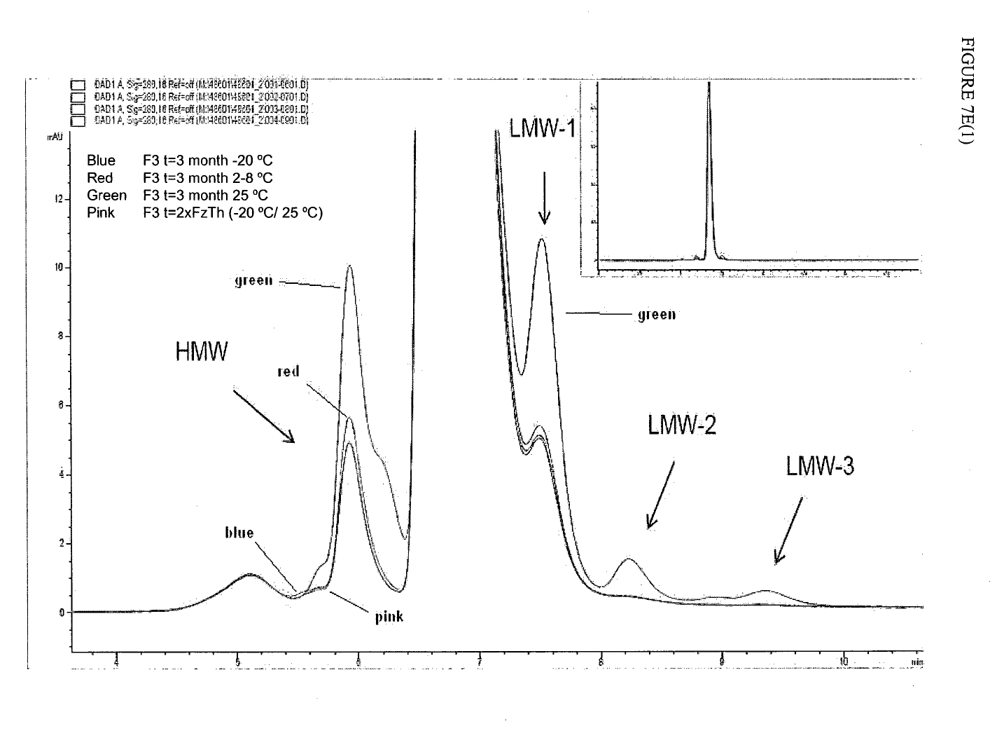

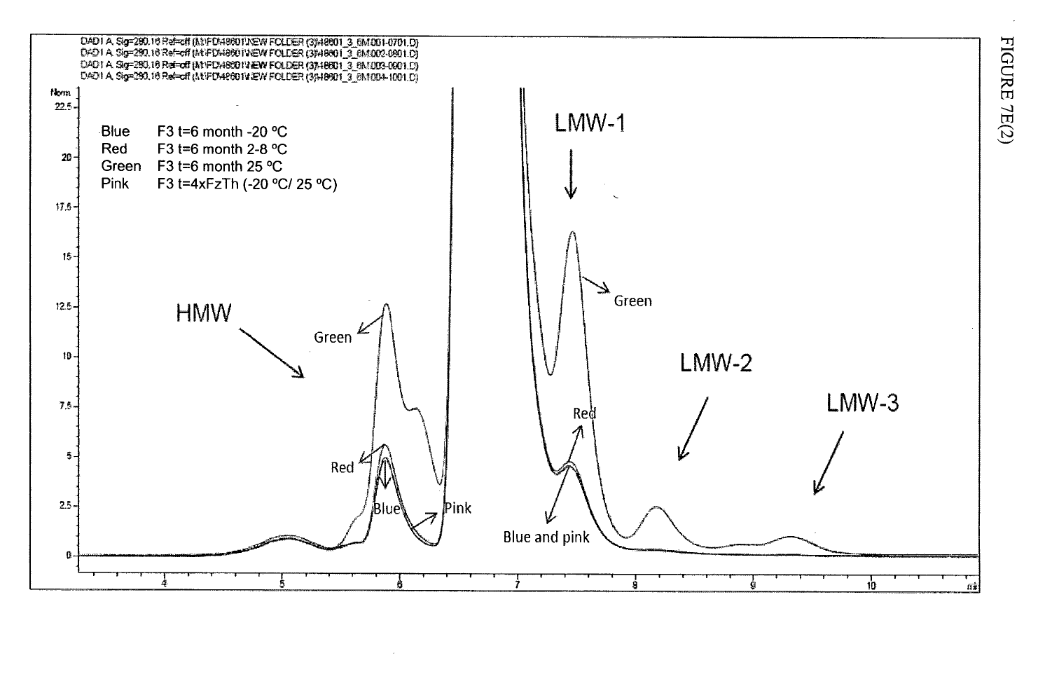

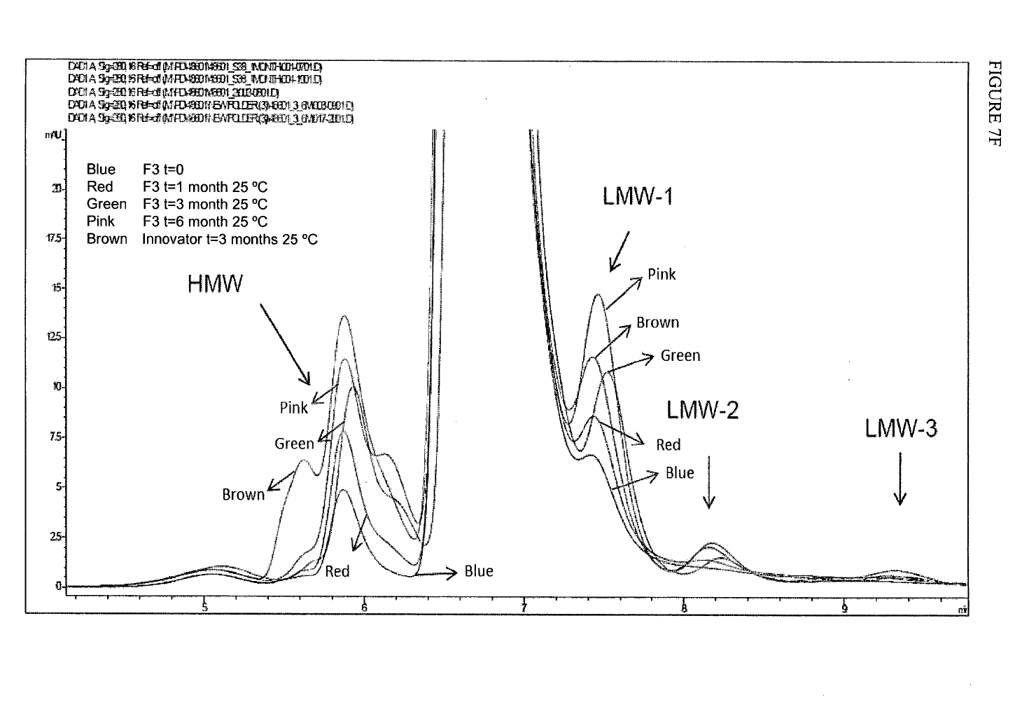

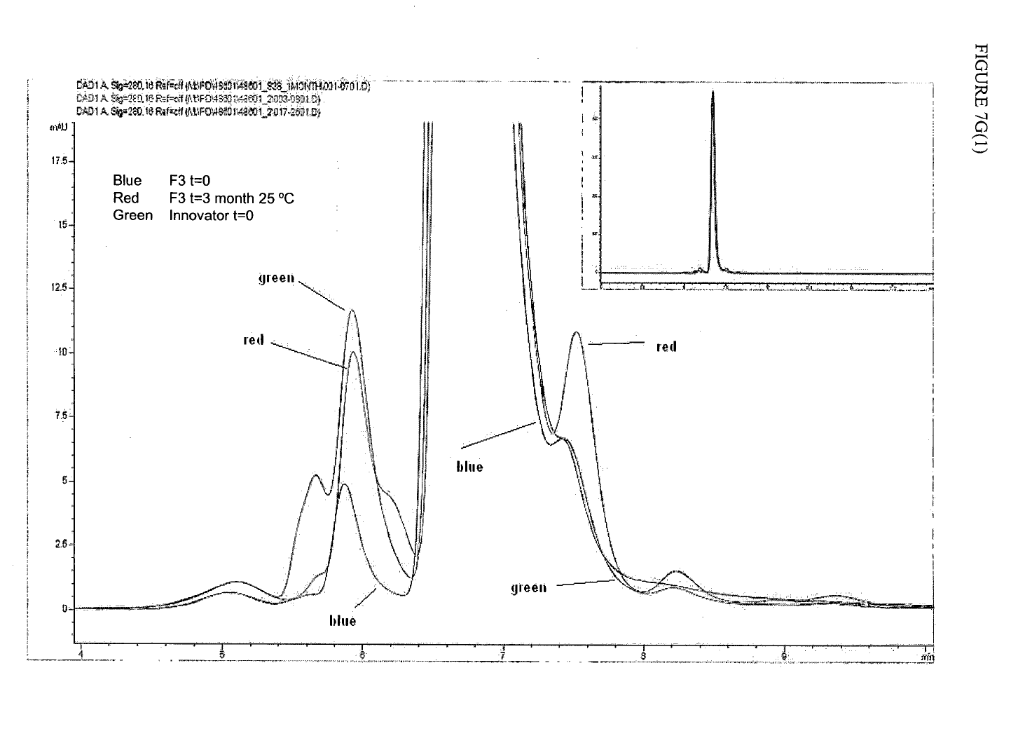

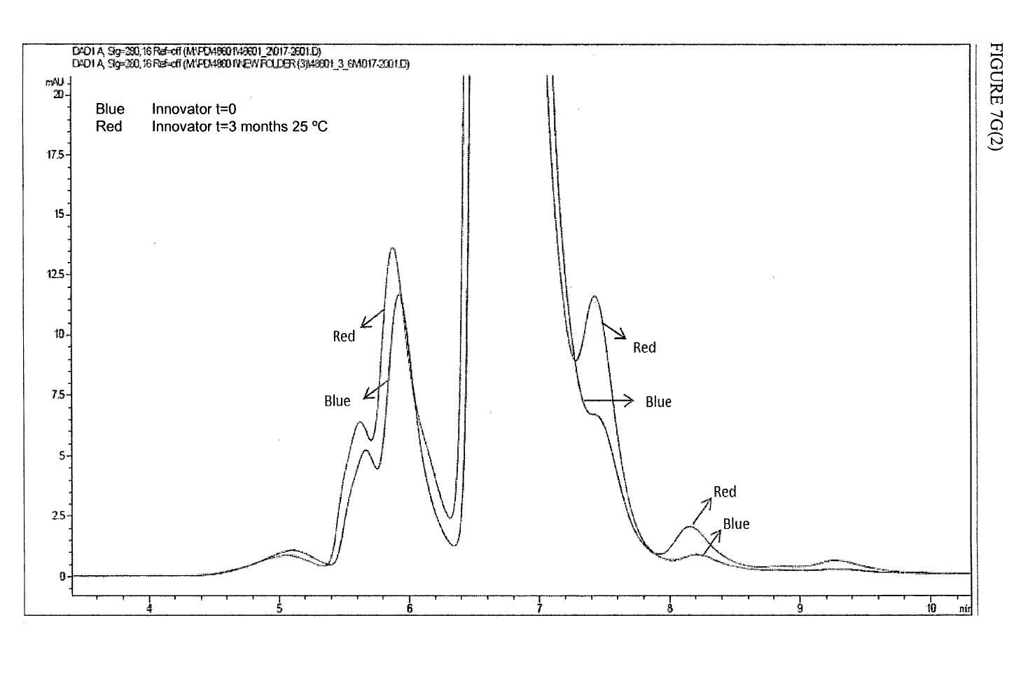

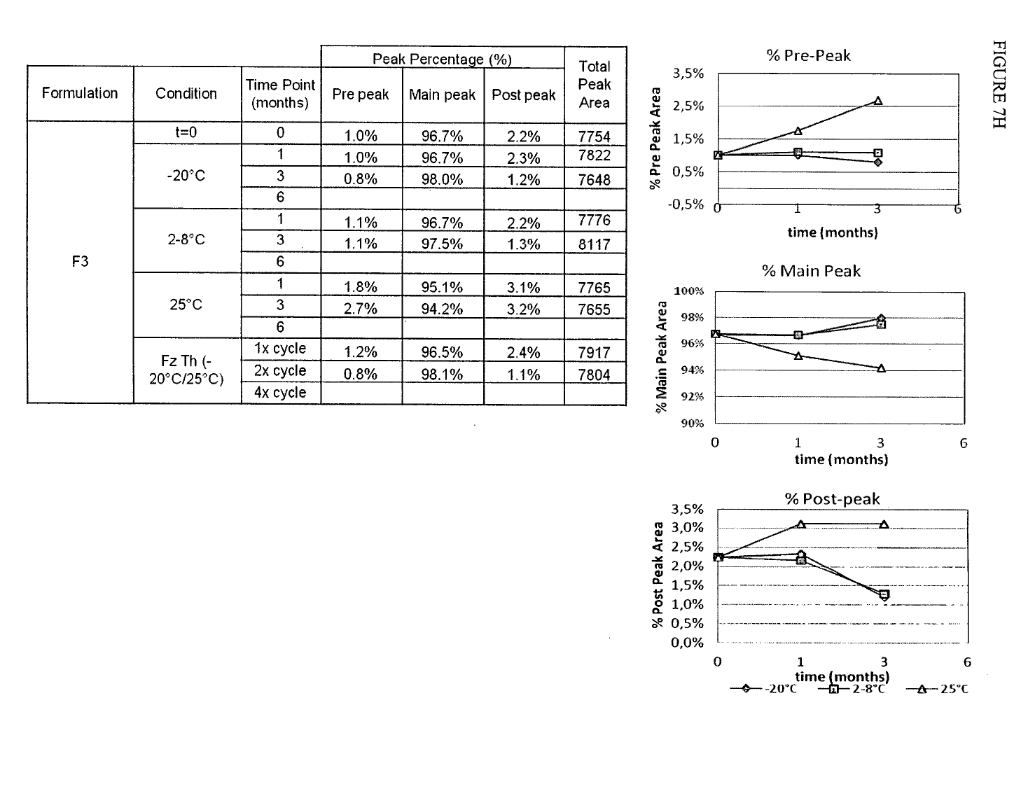

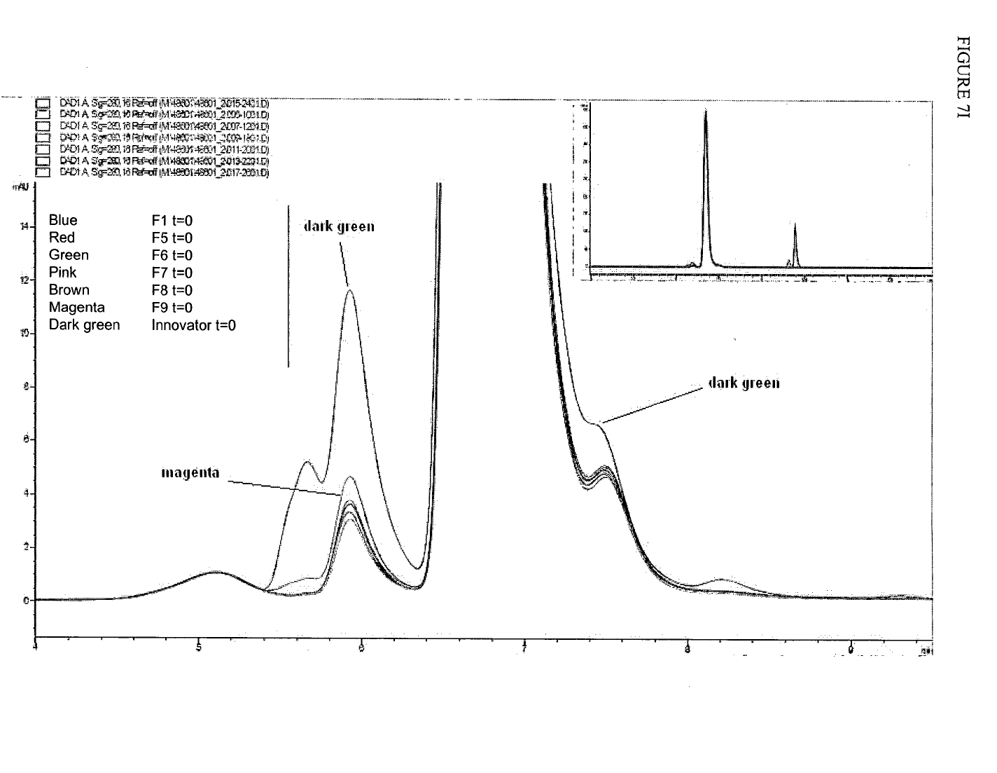

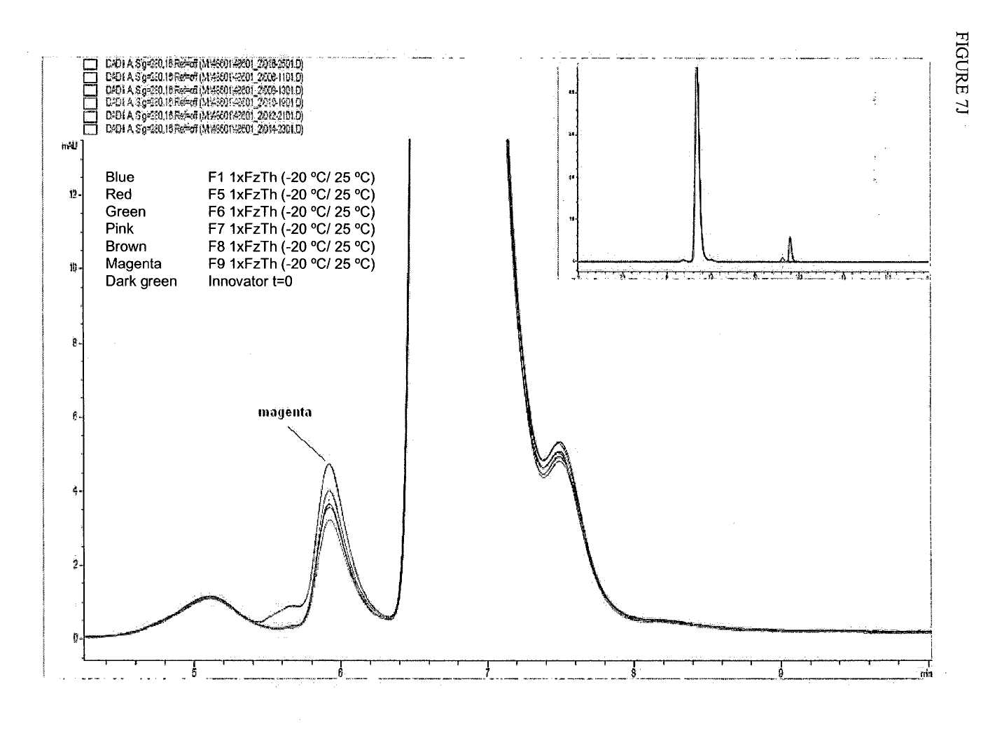

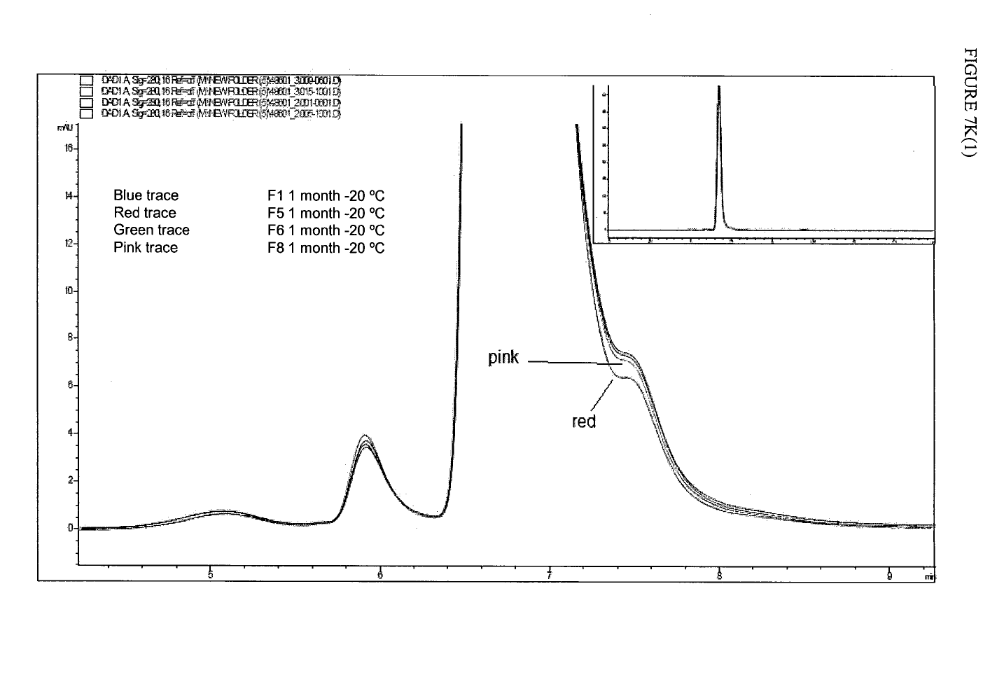

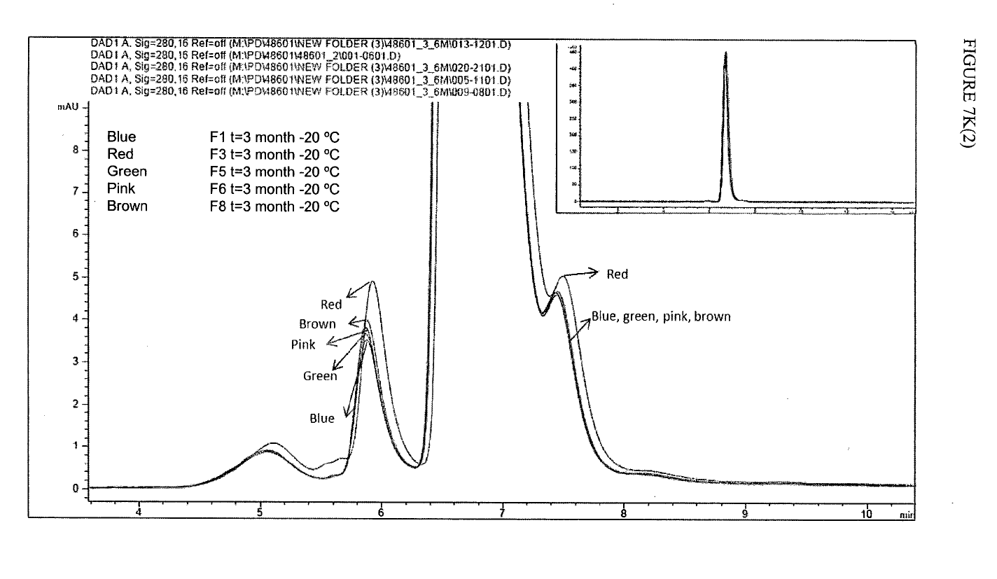

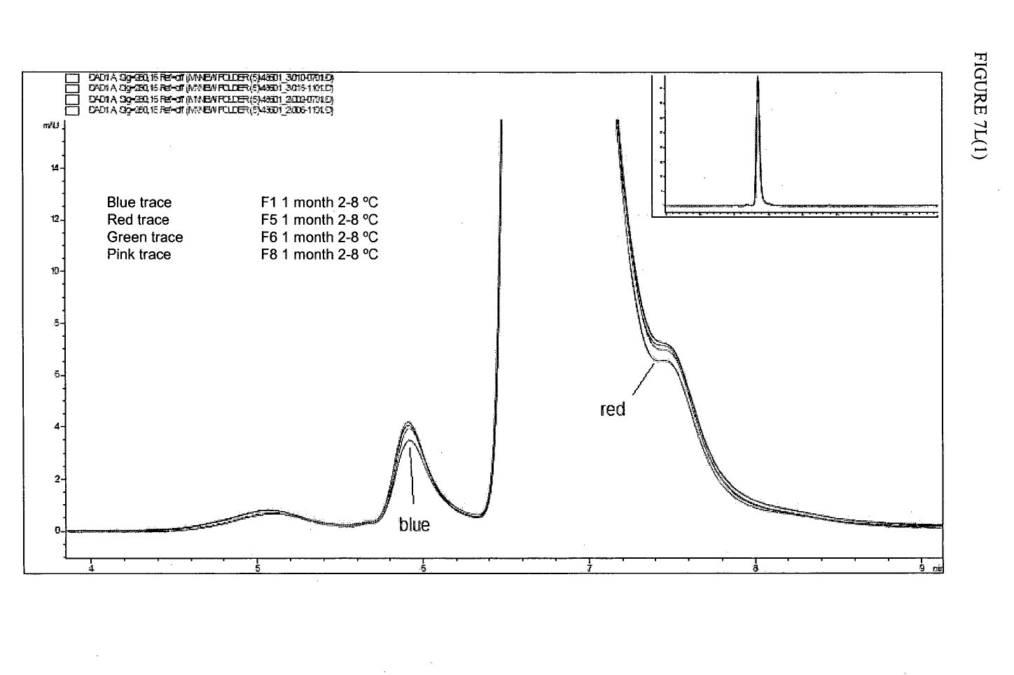

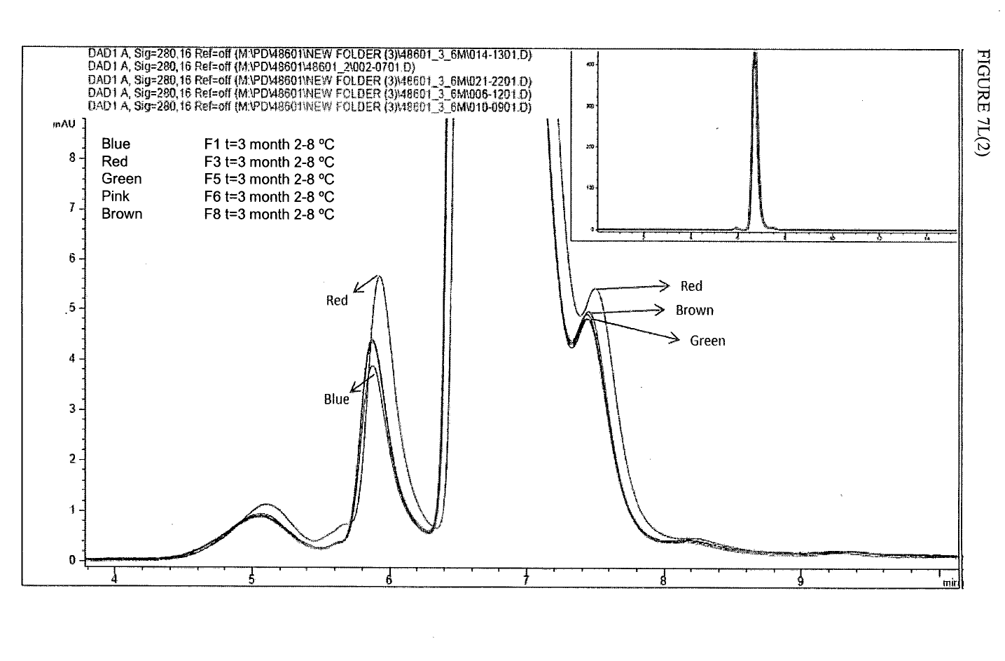

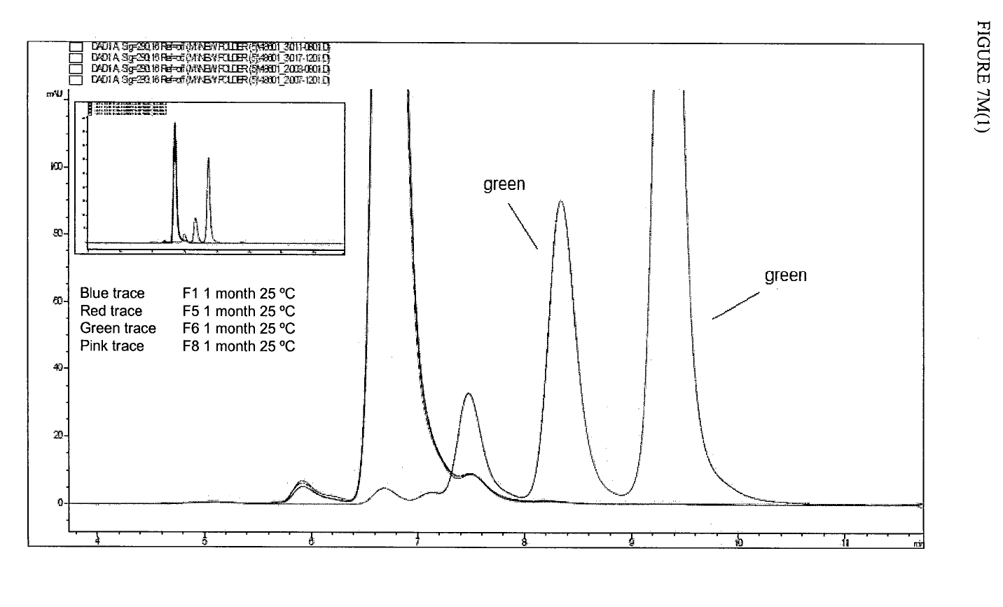

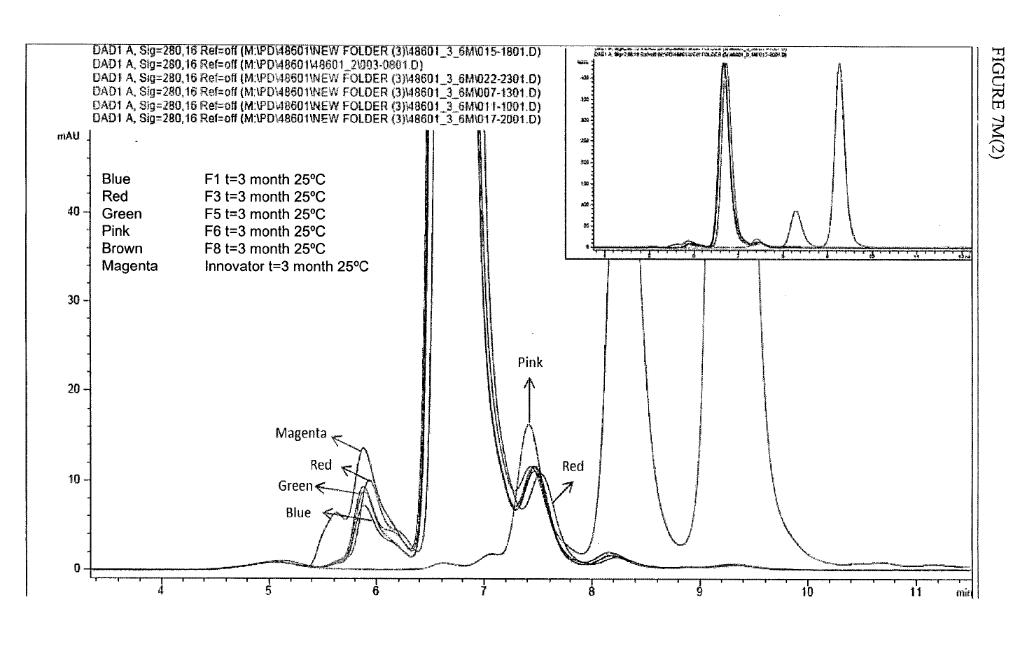

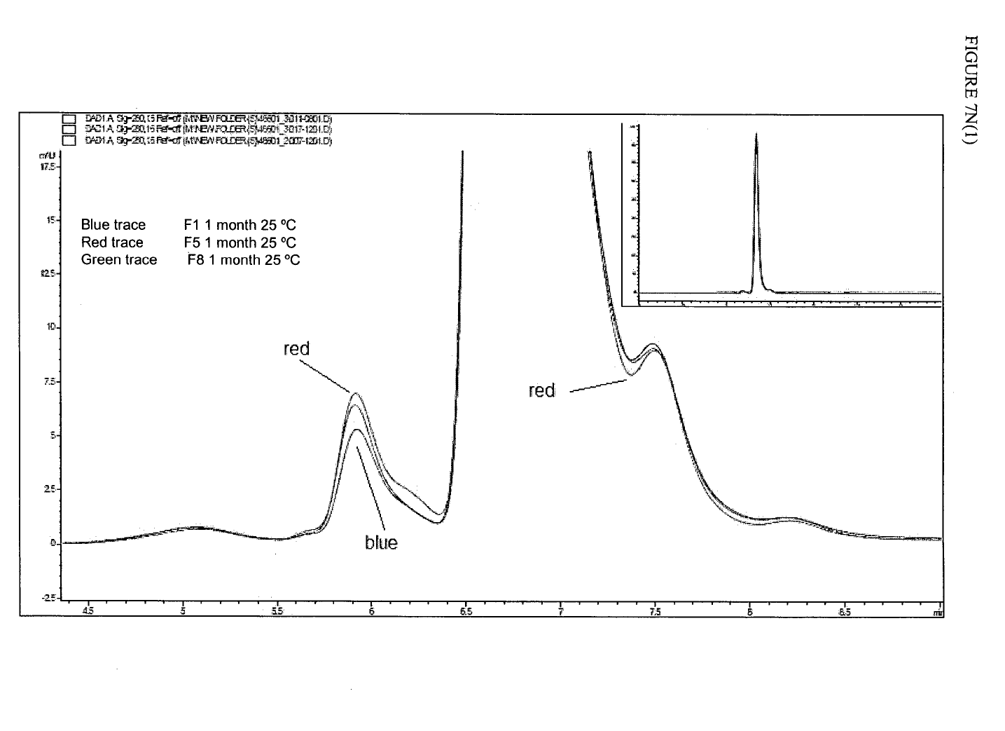

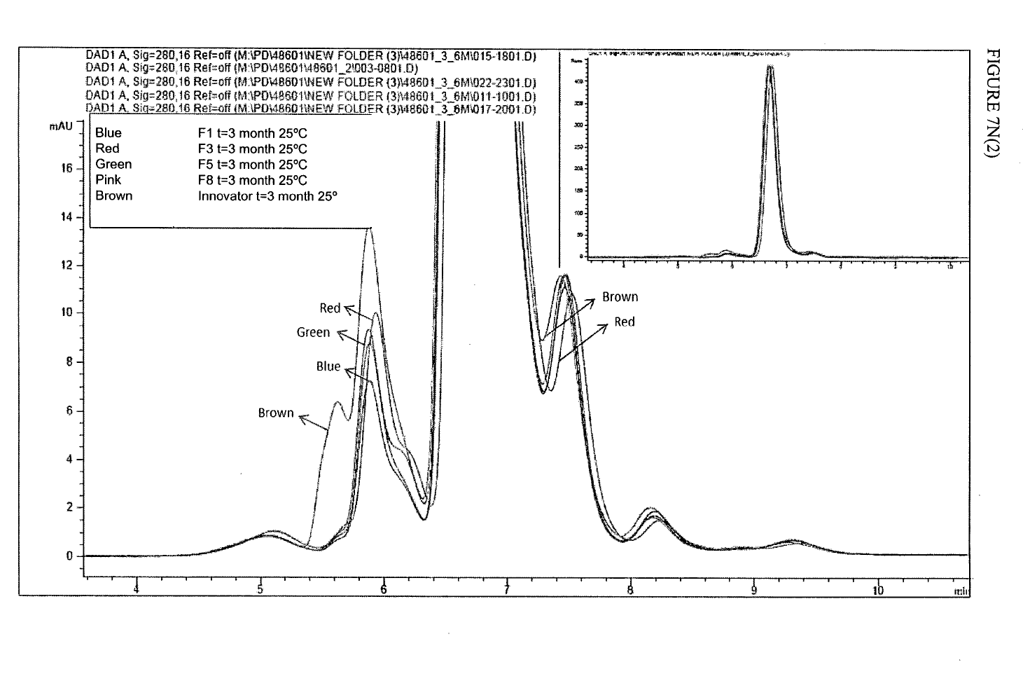

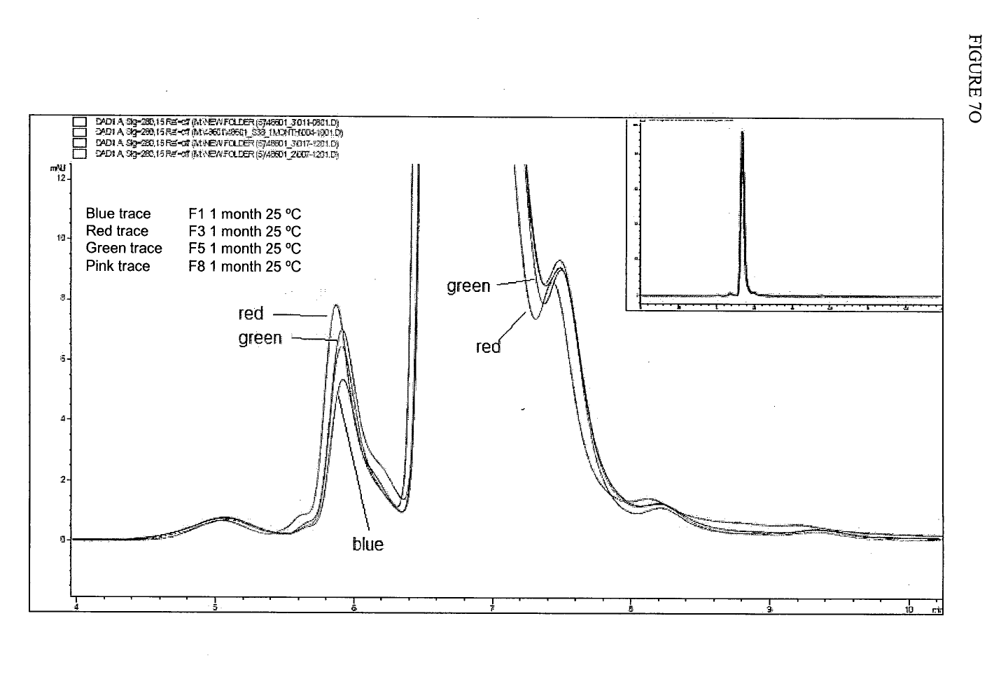

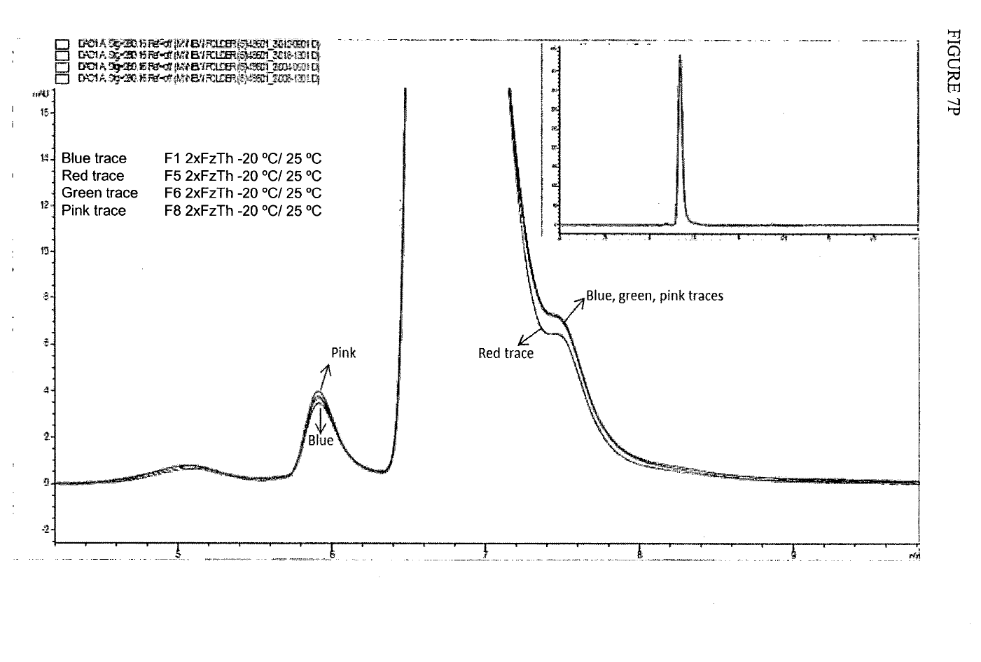

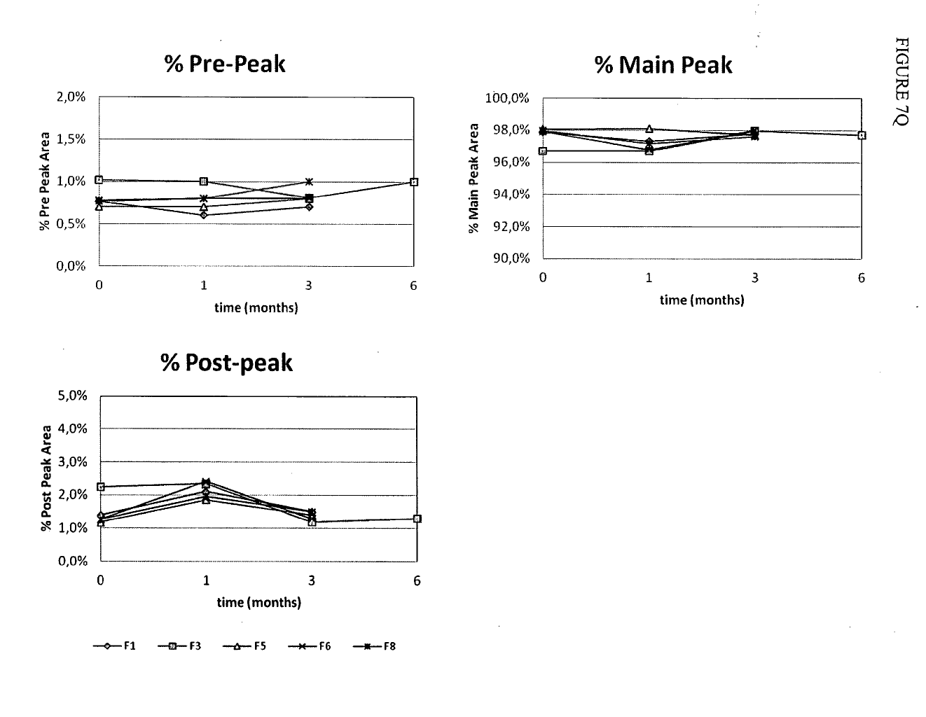

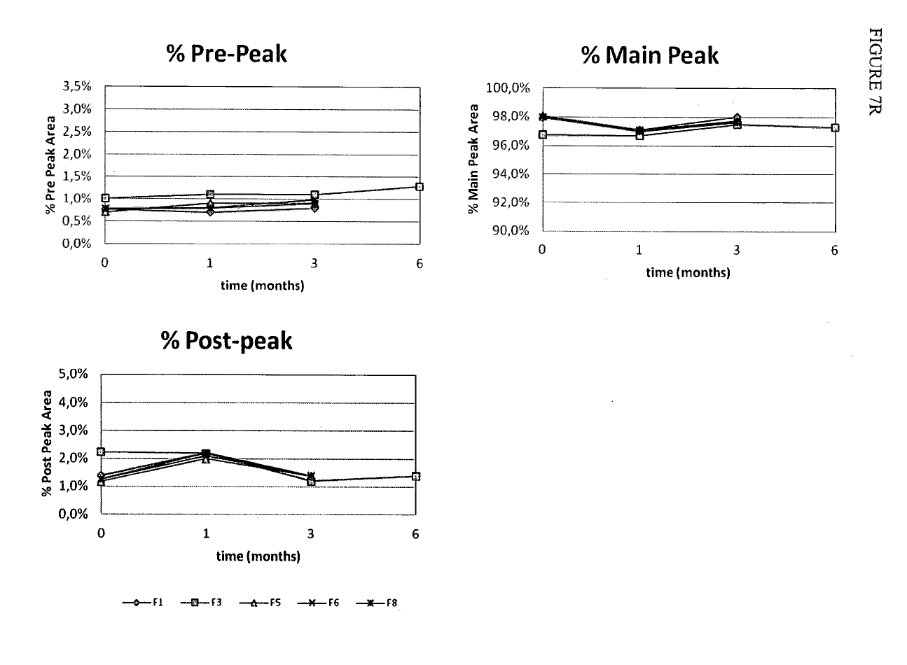

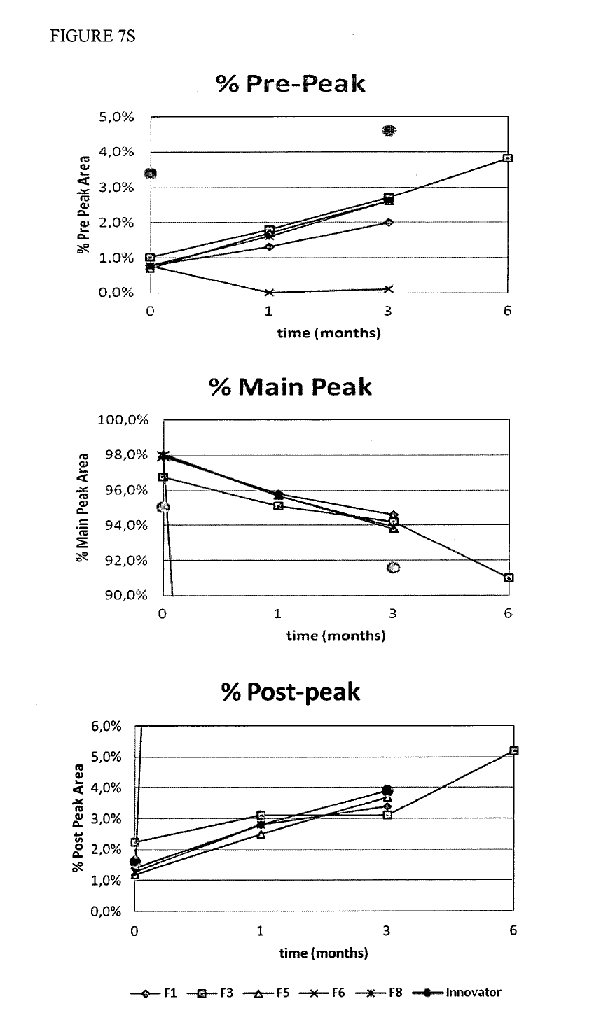

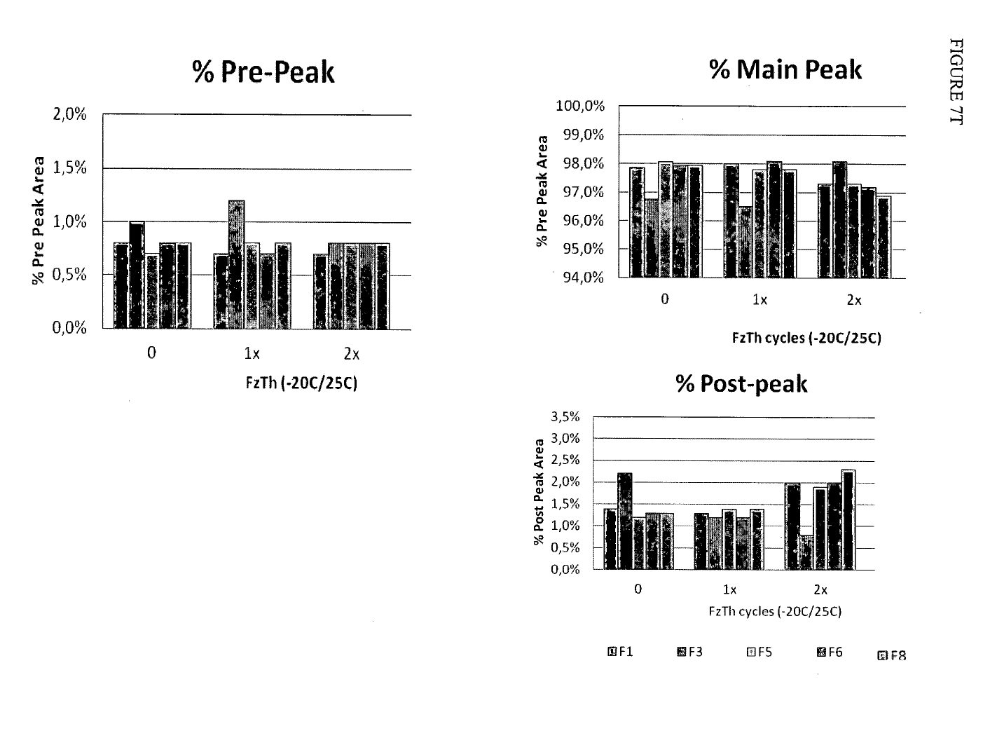

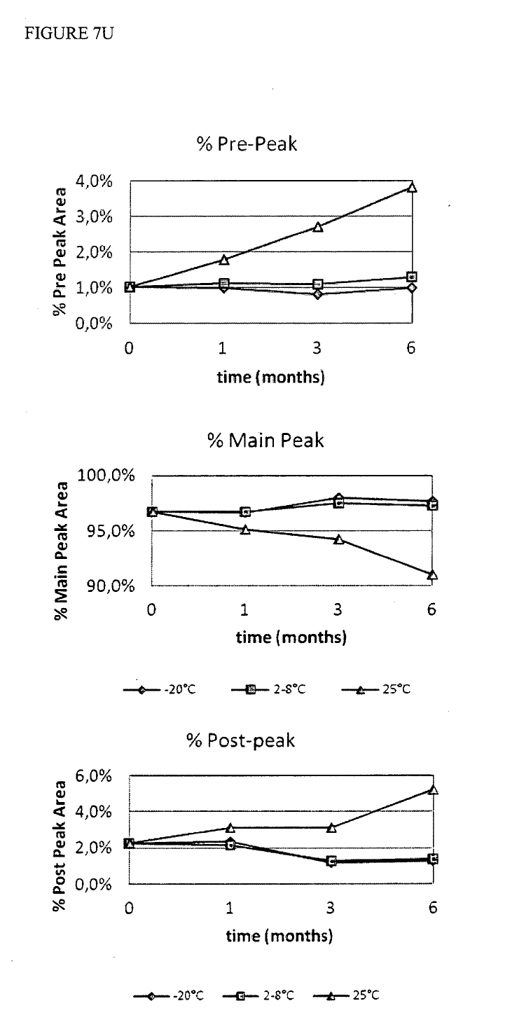

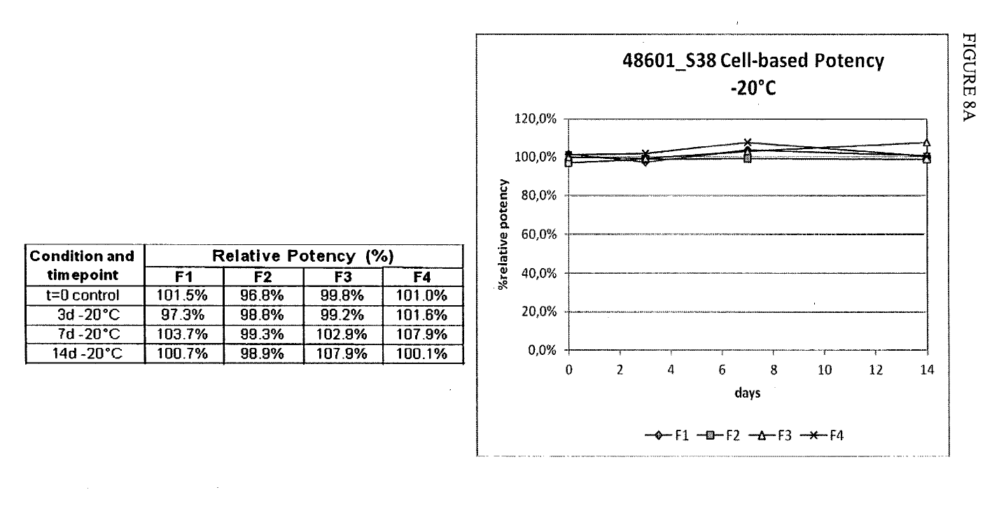

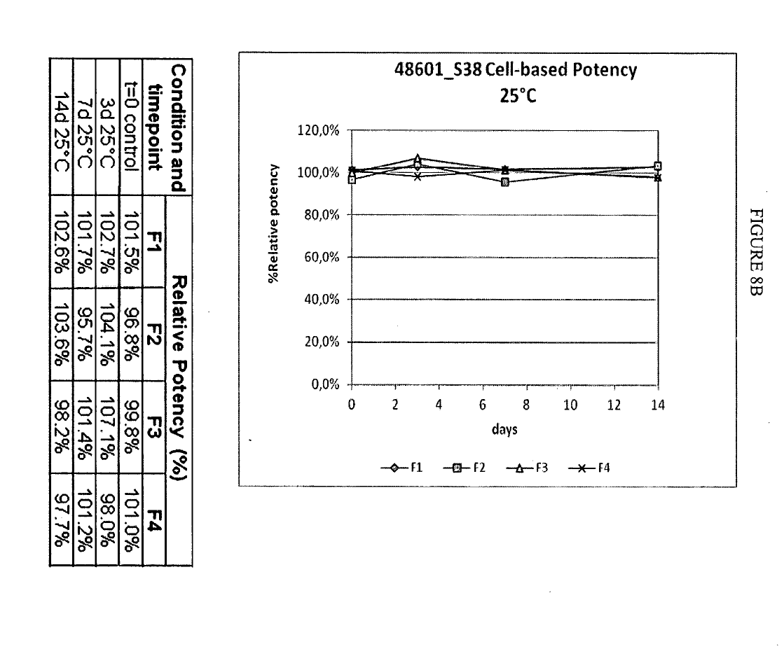

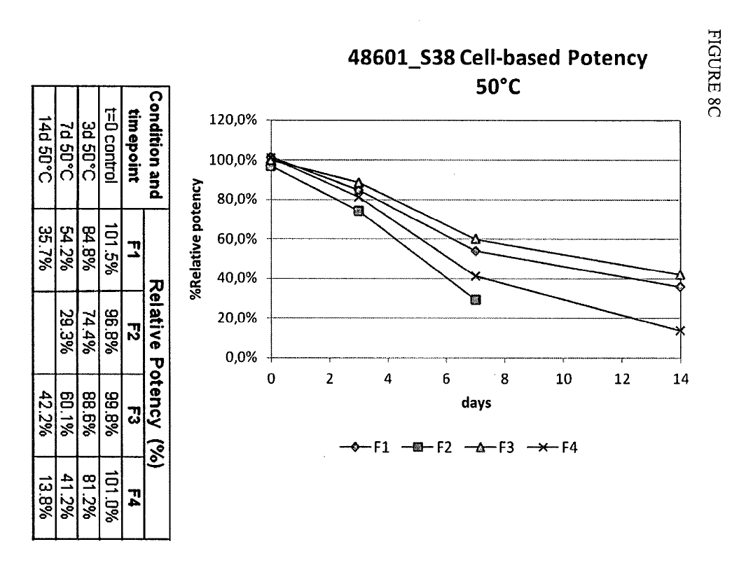

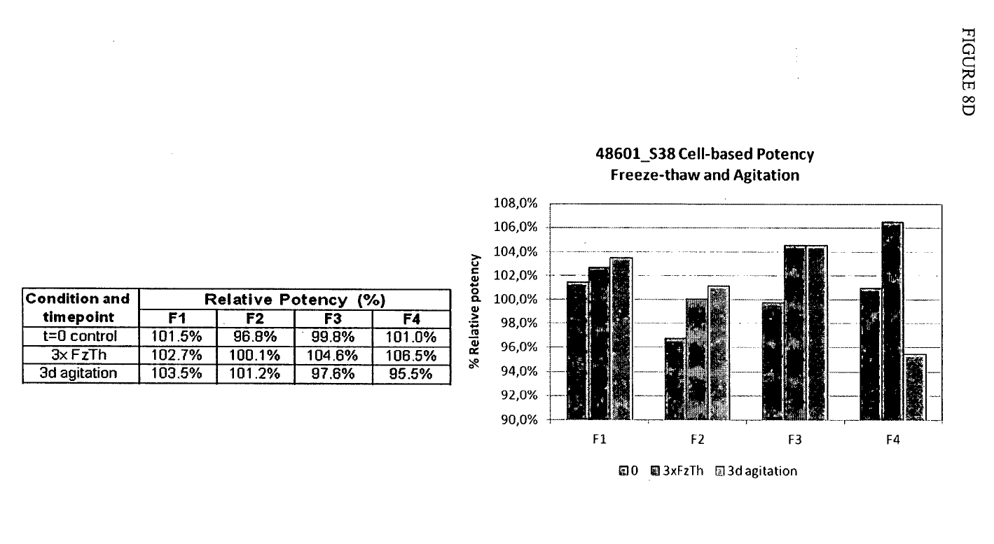

FIG. 7 shows the chromatograms of size exclusion HPLC in all formulations for all conditions: −20° C. (7A), 25° C. (7B), 50° C. (7C), 3 times freezing/thawing and 3 days in agitation (7D) at all timepoints. The peak percentages have been measured and represented in the tables.

Significant changes observed in all formulations for the 50° C. condition at all timepoints, with F2 performing worst overall with a dramatic increase in pre-peak aggregates as early as 3 days (26.3% and 22.7% respectively). F1 and F3 demonstrated a comparatively more moderate increase in pre-peak aggregation after 3 days at 50° C. (11.9% and 9.3% respectively), but increasing to >50% pre-peak aggregates for all four formulations after 14 days.

The 25° C. condition also resulted in slight changes for all formulations in both % main peak area and % pre-peak after 7 days, increasing further at 14 days, with F4 demonstrating the highest increase in pre-peak aggregates (0.5%) and F3 demonstrating the lowest increase in aggregation overall at this condition.

No significant changes were observed in any formulation when exposed to conditions of agitation and freeze-thaw or storage at −20° C. for up to 14 days.

FIG. 7E(1) shows the chromatogram of size exclusion HPLC in formulation F3 for t=3 months at −20° C., 2-8° C., 25° C. and 2 times freezing/thawing (2×FxTh) at −20° C./25° C. conditions.

A significant pre-peak aggregation and post-peak degradation is observed for this formulation exposed to 25° C. for 3 months as compared to all other conditions.

FIG. 7E(2) shows the chromatogram of size exclusion HPLC in formulation F3 for t=6 months at −20° C., 2-8° C., 25° C. and 4 times freezing/thawing (4×FxTh) at −20° C./25° C. conditions.

A significant pre-peak aggregation and post-peak degradation is observed for this formulation exposed to 25° C. for 6 months as compared to all other conditions after 6 months and after 4 cycles of freeze-thaw.

FIG. 7F shows the chromatogram of size exclusion HPLC in formulation F3 for t=0, 1, 3 and 6 months at 25° C. and in formulation Innovator at 25° C. after 3 months.

Formulation F3 demonstrates a further increase in pre-peak aggregates and post-peak aggregates as compared to the 1 and 3 months timepoints.

Innovator at 25° C. for 3 months demonstrates the highest % pre-peak overall and as compared to F3 at all other conditions tested, including 25° C. at 6 months.

FIG. 7G(1) shows the chromatogram of size exclusion HPLC in formulation F3 for t=0 and 3 months at 25° C. and compared to Innovator (control) at t=0.

Innovator (control) at t=0 presents significantly higher pre-peak aggregates overall, but less post-peak degradants than F3 after 3 months at 25° C.

FIG. 7G(2) shows the chromatogram of size exclusion HPLC in formulation Innovator at t=0 and 3 months at 25° C.

An increase in both pre-peak aggregates and post-peak degradants are observed after 3 months at 25° C. for Innovator as compared to Innovator at t=0.

FIG. 7H provides the tabular results for a longer term study with size exclusion HPLC in formulation F3 for t=0 at −20° C., 2-8° C., 25° C. and 1 and 2 times freezing/thawing (1× and 2×FxTh) at −20° C./25° C. conditions.

Formulation F3 demonstrates a significant further increase in pre-peak aggregates (0.9% from t=1 month at 25° C.) and a slight further increase in post-peak degradants (0.1% further increase in LMW-1 peak from 1 month).

FIG. 7I shows the chromatogram of size exclusion HPLC in formulations F1, F5, F6, F7, F8, F9 and Innovator (control) at t=0.

All these formulations present at t=0 comparable chromatographic profiles.

Formulation F9 at t=0 presents a slightly higher pre-peak than F1, F6, F6, F7 and F8.

Innovator (control) at t=0 presents both significantly higher % pre- and post-peak as compared to F1, F5, F6, F7, F8 and F9 at t=0.

FIG. 7J shows the chromatogram of size exclusion HPLC in formulations F1, F5, F6, F7, F8 and F9 after 1 cycle freezing/thawing at −20° C./25° C.

Formulations F1, F5, F6, F7 and F8 are comparable after 1 cycle of freeze-thaw, with F9 demonstrating slightly higher % pre-peak (however with no further increase from t=0).

The following table provides the results for a longer term study with size exclusion HPLC in formulations F1, F5, F6, F7, F8 and F9 and Innovator (control) for t=0 and after 1 cycle freezing/thawing (1×FxTh) at −20° C./25° C. conditions.

| | | % | % | % | Total Peak |
|---|---|---|---|---|---|
| Formulation | Condition | Pre-Peak | Main Peak | Post Peak | Area |
| F1 | t = 0 | 0.8% | 97.9% | 1.4% | 7205 |
| | 1x Fz Th | 0.7% | 98.0% | 1.3% | 7873 |
| F5 | t = 0 | 0.7% | 98.1% | 1.2% | 7627 |
| | 1x FzTh | 0.8% | 97.8% | 1.4% | 8054 |
| F6 | t = 0 | 0.8% | 97.9% | 1.3% | 7607 |
| | 1x FzTh | 0.7% | 98.1% | 1.2% | 7473 |
| F7 | t = 0 | 0.7% | 97.9% | 1.4% | 7135 |
| | 1x FzTh | 0.6% | 98.0% | 1.4% | 7569 |
| F8 | t = 0 | 0.8% | 98.0% | 1.3% | 7242 |
| | 1x FzTh | 0.8% | 97.8% | 1.4% | 7215 |
| F9 | t = 0 | 1.0% | 97.7% | 1.3% | 7443 |
| | 1x FzTh | 1.0% | 97 8% | 1.2% | 7507 |
| Innovator | t = 0 | 3.4% | 95.0% | 1.6% | 7677 |

The control (innovator) presents the highest % pre-peak aggregates as compared to F1, F5, F6, F7, F8 and F9 at t=0.

FIG. 7K(1) shows the chromatogram of size exclusion HPLC in formulations F1, F5, F6, F8, for t=1 month at −20° C.

No significant differences between formulations are shown after 1 month at −20° C. storage condition. Only a slightly less post peak is observed for formulation F5.

FIG. 7K(2) shows the chromatogram of size exclusion HPLC in formulations F1, F3, F5, F6, F8, for t=3 months at −20° C.

No significant differences between formulations are shown for F1, F5, F6 and F8 after 3 months at −20° C. storage condition. Higher pre- and post-peak observed for F3 after 3 months at −20° C. and as compared to all other formulations.

FIG. 7L(1) shows the chromatogram of size exclusion HPLC in formulations F1, F5, F6, F8, for t=1 month at 2-8° C.

No significant differences between formulations are shown after 1 month at 2-8° C. storage condition. A slightly less post peak is observed for formulation F5.

FIG. 7L(2) shows the chromatogram of size exclusion HPLC in formulations F1, F3, F5, F6, F8, for t=3 months at 2-8° C.

No significant differences between formulations are shown after 3 months at 2-8° C. storage condition. Higher pre- and post-peak observed for F3 after 3 months at 2-8° C. and as compared to all other formulations.

FIG. 7M(1) shows the chromatogram of size exclusion HPLC in formulations F1, F5, F6, F8, for t=1 month at 25° C.

Dramatic changes are observed in F6 after 1 month at 25° C. condition, with a complete loss of main peak resulting in post peak degradation. No significant changes in all other formulations (F1, F5, F8) are observed after 1 month at 25° C.

FIG. 7M(2) shows the chromatogram of size exclusion HPLC in formulations F1, F3, F5, F6, F8 and Innovator for t=3 months at 25° C.

No significant differences between formulations are shown for F1, F3, F5, F6, F8 after 3 months at 25° C. storage condition, with slightly less post peak observed for F5. Innovator demonstrates the highest pre- and post-peak observed for F3 after 3 months at 25° C. F6 presents with a dramatic change in profile, with a complete loss of main peak.

FIG. 7N(1) shows the chromatogram of size exclusion HPLC in formulations F1, F5 and F8, for t=1 month at 25° C.

FIG. 7N(2) shows the chromatogram of size exclusion HPLC in formulations F1, F3, F5, F8 and Innovator for t=3 month at 25° C.

No significant differences between F1, F3, F5 and F8 formulations after 3 months at 25° C. storage condition. Innovator shows significant pre-peak aggregates and post-peak degradants as compared to all other formulations.

FIG. 7O shows the chromatogram of size exclusion HPLC in formulations F1, F3, F5 and F8, for t=1 month at 25° C.

Formulation F3 presents the highest % pre-peak aggregates after 1 month at 25° C.

FIG. 7P shows the chromatogram of size exclusion HPLC in formulations F1, F5, F6 and F8 after 2 cycles freezing/thawing at −20° C./25° C.

No significant differences between formulations are shown after 2 cycles of freeze-thaw at −20° C./25° C. Only a slightly less post peak is observed for formulation F5.

The following table provides the results for a longer term study with size exclusion HPLC in formulation F1 for t=0, 1 and 3 months at −20° C., 2-8° C. and 25° C. storage conditions and after 1, 2 and 4 cycles freezing/thawing (1×, 2× and 4×FxTh) at −20° C./25° C. conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| F1 | t = 0 | 0 | 0.8% | 97.9% | 1.4% | 7206 |
| | −20° C. | 1 | 0.6% | 97.3% | 2.1% | 7512 |
| | | 3 | 0.7% | 97.8% | 1.5% | 7380 |
| | 2-8° C. | 1 | 0.7% | 97.1% | 2.2% | 7493 |
| | | 3 | 0.8% | 98.0% | 1.2% | 7367 |
| | 25° C. | 1 | 1.3% | 95.8% | 2.8% | 7502 |
| | | 3 | 2.0% | 94.6% | 3.4% | 7349 |
| | Fz Th | 1x cycle | 0.7% | 98.0% | 1.3% | 7874 |
| | (−20° C./ | 2x cycle | 0.7% | 97.3% | 2.0% | 7539 |
| | 25° C.) | 4x cycle | 0.7% | 97.9% | 1.3% | 7710 |

The following table provides the results for a longer term study with size exclusion HPLC in formulation F5 for t=0, 1 and 3 months at −20° C., 2-8° C. and 25° C. storage conditions and after 1, 2 and 4 cycles freezing/thawing (1×, 2× and 4×FxTh) at −20° C./25° C. conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| F5 | t = 0 | 0 | 0.7% | 98.1% | 1.2% | 7628 |
| | −20° C. | 1 | 0.7% | 97.4% | 1.9% | 7602 |
| | | 3 | 0.8% | 97.7% | 1.4% | 7440 |
| | 2-8° C. | 1 | 0.9% | 97.1% | 2.0% | 7606 |
| | | 3 | 0.9% | 97.7% | 1.4% | 7502 |
| | 25° C. | 1 | 1.7% | 95.7% | 2.5% | 7643 |
| | | 3 | 2.6% | 93.8% | 3.7% | 7682 |
| | Fz Th | 1x cycle | 0.8% | 97.8% | 1.4% | 8054 |
| | (−20° C./ | 2x cycle | 0.8% | 97.3% | 1.9% | 7610 |
| | 25° C.) | 4x cycle | 0.8% | 97.8% | 1.4% | 7426 |

The following table provides the results for a longer term study with size exclusion HPLC in formulation F6 for t=0, 1 and 3 months at −20° C., 2.8° C. and 25° C. storage conditions and after 1, 2 and 4 cycles freezing/thawing (1×, 2× and 4×FxTh) at −20° C./25° C. conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| F6 | t = 0 | 0 | 0.8% | 97.9% | 1.3% | 7607 |
| | −20° C. | 1 | 0.8% | 96.8% | 2.4% | 7775 |
| | | 3 | 0.8% | 98.0% | 1.3% | 7448 |
| | 2-8° C. | 1 | 0.8% | 97.1% | 2.1% | 7714 |
| | | 3 | 1.0% | 97.6% | 1.4% | 7399 |
| | 25° C. | 1 | 0.0% | 1.1% | 98.9% | 7693 |
| | | 3 | 0.1% | 0.6% | 99.3% | 7368 |
| | Fz Th | 1x cycle | 0.7% | 98.1% | 1.2% | 7474 |
| | (−20° C./ | 2x cycle | 0.8% | 97.2% | 2.0% | 7627 |
| | 25° C.) | 4x cycle | 0.8% | 97.9% | 1.4% | 7554 |

The following table provides the results for a longer term study with size exclusion HPLC in formulation F8 for t=0, 1 and 3 months at −20° C., 2-8° C. and 25° C. storage conditions and after 1, 2 and 4 cycles freezing/thawing (1×, 2× and 4×FxTh) at −20° C./25° C. conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| F8 | t = 0 | 0 | 1.0% | 96.7% | 2.2% | 7754 |
| | −20° C. | 1 | 0.8% | 97.2% | 2.0% | 7550 |
| | | 3 | 1.0% | 97.6% | 1.5% | 7490 |
| | 2-8° C. | 1 | 0.8% | 97.0% | 2.2% | 7453 |
| | | 3 | 0.9% | 97.6% | 1.4% | 7539 |
| | 25° C. | 1 | 1.6% | 95.7% | 2.8% | 7489 |
| | | 3 | 2.3% | 93.9% | 3.9% | 7459 |
| | Fz Th | 1x cycle | 1.2% | 96.5% | 2.4% | 7917 |
| | (−20° C./ | 2x cycle | 0.8% | 96.9% | 2.3% | 7523 |
| | 25° C.) | 4x cycle | 0.7% | 97.8% | 1.5% | 7379 |

FIGS. 7Q, 7R and 7S show the graphical summary of chromatograms of size exclusion HPLC in formulations F1, F3, F5, F6 and F8 for conditions: −20° C. (FIG. 7Q), 2-8° C. (7R) and 25° C. (7S) at time points up to 6 months for formulation F3 and up to 3 month for formulations F1, F5, F6 and F8. The peak percentages have been measured and represented (% pre-peak, % main-peak and % post-peak).

FIG. 7T show the graphical summary of chromatograms of size exclusion HPLC in formulations F1, F3, F5, F6 and F8 at t=0 and after 1 and 2 cycles freezing/thawing (1× and 2×FxTh) at −20° C./25° C. conditions. The peak percentages have been measured and represented (% pre-peak, % main-peak and % post-peak). Bars are indicated in the following order of formulation: F1, F3, F5, F6 and F8 for each condition (i.e. t=0, 1×FxTh or 2×FxTh).

The following table provides the results for a longer term study with size exclusion HPLC in formulation Innovator for t=0 at 25° C. storage conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| Innovator | t = 0 | 0 | 3.4% | 95.0% | 1.6% | 7677 |
| | 25° C. | 3 | 4.6% | 91.6% | 3.9% | 7537 |

The following table provides the results for a longer term study with size exclusion HPLC in formulation F3 for t=0, 1, 3 and 6 months at −20° C., 2-8° C. and 25° C. storage conditions and after 1, 2 and 4 cycles freezing/thawing (1×, 2× and 4×FxTh) at −20° C./25° C. conditions.

| | | | Peak Percentage (%) | | | Total |
|---|---|---|---|---|---|---|
| | | Time Point | Pre | Main | Post | Peak |
| Formulation | Condition | (months) | peak | peak | peak | Area |
| F3 | t = 0 | 0 | 1.0% | 96.7% | 2.2% | 7754 |
| | −20° C. | 1 | 1.0% | 96.7% | 2.3% | 7822 |
| | | 3 | 0.8% | 98.0% | 1.2% | 7648 |
| | | 6 | 1.0% | 97.7% | 1.3% | 7308 |
| | 2-8° C. | 1 | 1.1% | 96.7% | 2.2% | 7776 |
| | | 3 | 1.1% | 97.5% | 1.3% | 8117 |
| | | 6 | 1.3% | 97.3% | 1.4% | 7371 |
| | 25° C. | 1 | 1.8% | 95.1% | 3.1%* | 7765 |
| | | 3 | 2.7% | 94.2% | 3.1% | 7655 |
| | | 6 | 3.8% | 91.0% | 5.2% | 7250 |
| | Fz Th | 1x cycle | 1.2% | 96.5% | 2.4% | 7917 |
| | (−20° C./ | 2x cycle | 0.8% | 98.1% | 1.1% | 7804 |
| | 25° C.) | 4x cycle | 1.1% | 97.4% | 1.4% | 7179 |

The results are shown in FIG. 7U. F3 demonstrates significant further increase in pre-peak aggregates at 6 months (1.1% increase from t=3 months at 25° C.) and a slight further increase in post-peak degradants (2.1% further increase in post-peak from 3 months).

### Cell Based Potency Assay

### Approach:

For Shorter Timepoints (0, 3, 7 and 14 Days)
Samples were tested two batches (after t=0 and t=3 days (d) and after t=7 and t=14 d time points).
All the samples were tested in the bioassay once by a single analyst, except the control sample which was tested on each of the six (6) testing days.
Absorbance measurements at A280 nm were taken to determine the accurate concentration of the primary dilutions and subsequent sample dilution.
Overall assay performance was acceptable. Three (3) out of 106 dose response curves (from 53 plates) needed to have one well at up to 2 different concentrations masked to meet the well-to-well variability assay criteria
Well-to-well variability %/CV≦20%
Assay window (D/A)≧6
R²≧0.98

The relative potency of 47 test samples was measured once and a control was measured six (6) different times. The mean relative potency of the control was 100.2% with 95% CI from 96.9% to 103.6%.
The assay variability (% GCV) for the six independent measurements of the control was 3.2%. The low assay variability of this method demonstrated that the relative potency values of test samples obtained from single measurement was acceptable.
Based on single measurements, the majority of the test samples had relative potencies close to 100% (comparable to that of the reference standard).
Test samples started losing potency when stored at elevated temperature (50° C.) for three (3) days and the potency declined at later time points.

For Longer Timepoints (3 Months and 6 Months)
Samples were tested in one batch (including t=6 months (F3) and t=3 months (for all other samples and conditions).
All the samples were tested in the bioassay once by a single analyst. The reference standard used is E16 ADS Lot DC-4168-85.
Absorbance measurements at A280 nm were taken to determine the accurate concentration of the primary dilutions and subsequent sample dilution.
Overall assay performance was acceptable. All of the dose response curves (12 dose response curves from 6 plates) meet the well-to-well variability assay criteria without masking any wells. The assay acceptance criteria specified in TME 0498-01 is as follows:
   Well-to well variability % CV≦20%
   Assay window (D/A)≧6
   R≧0.98
Assay window for the dose response curves in the assay was ranged from ˜4 to 4.5. All the key parameters (A, B, C and D) of the dose response curves are within the normal range of historical data. It has been shown before that smaller assay window (>3) would not comprise the assay accuracy and therefore the results of this assay were accepted.

In this case, the data was analyzed using Softmax Pro v5.2 to verify the assay acceptance criteria and, if necessary, to mask wells.

### Cell Based Bioassay Results:

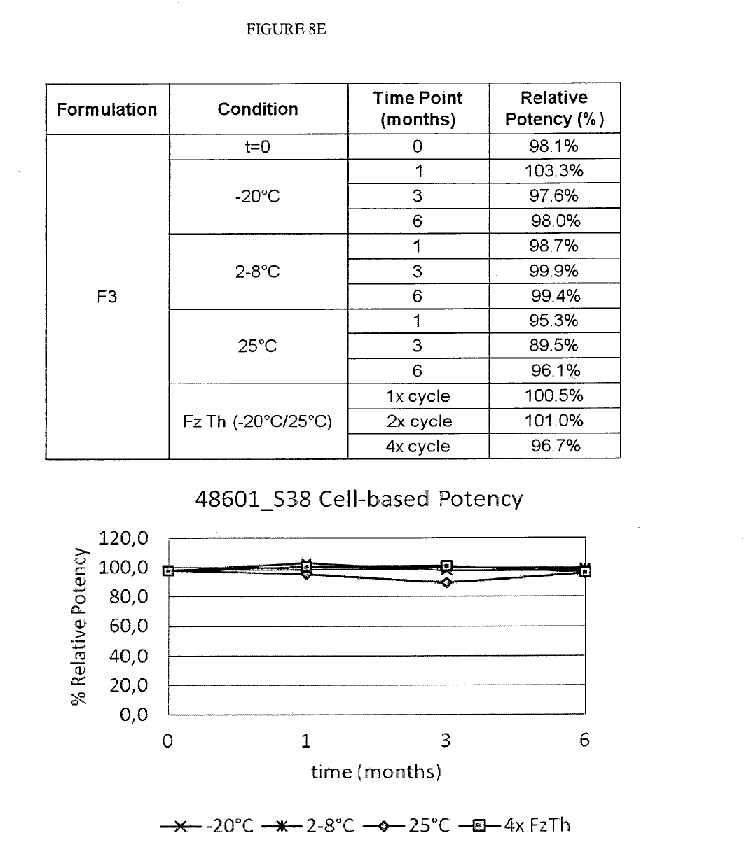

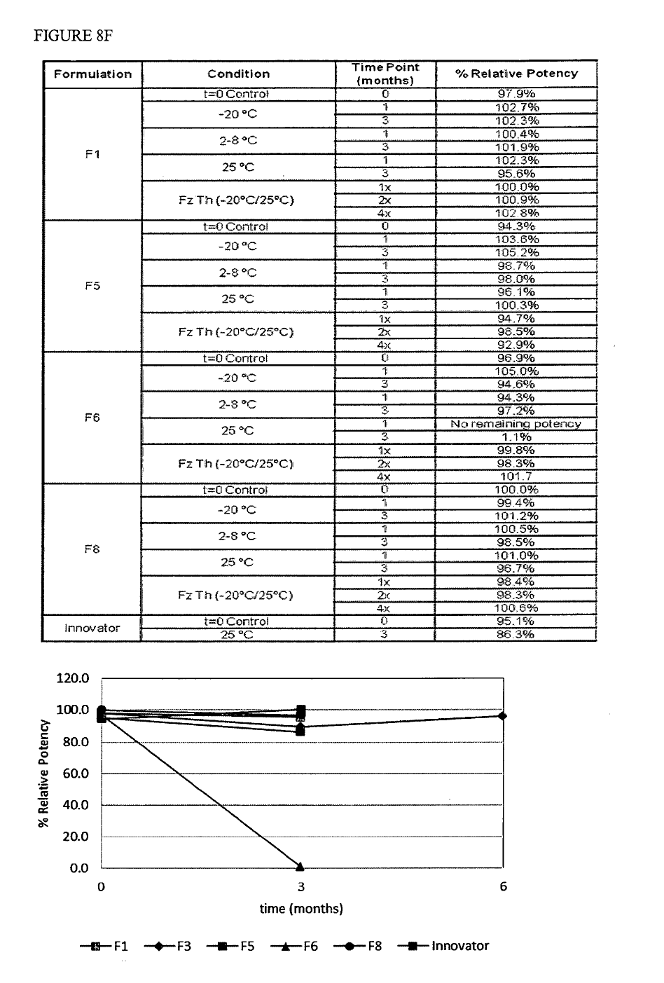

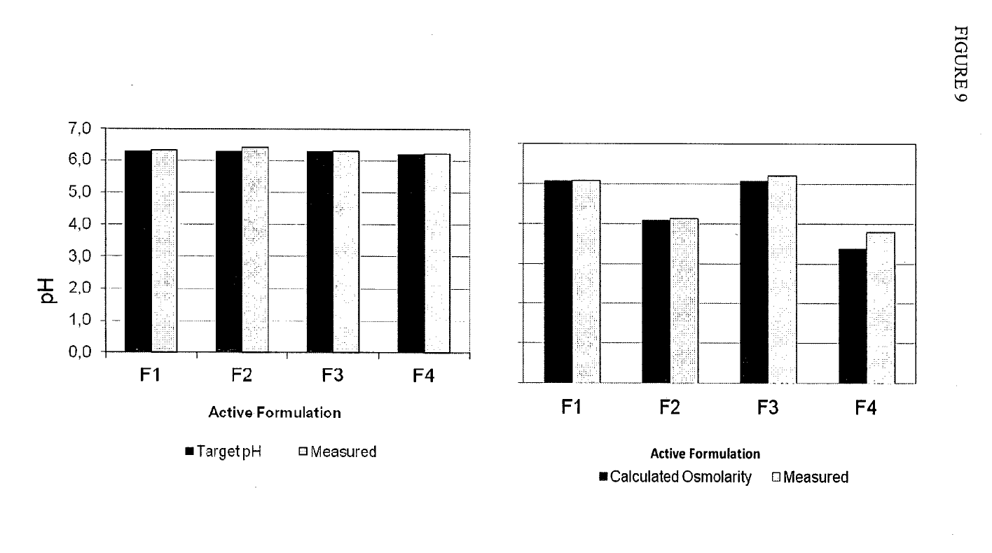

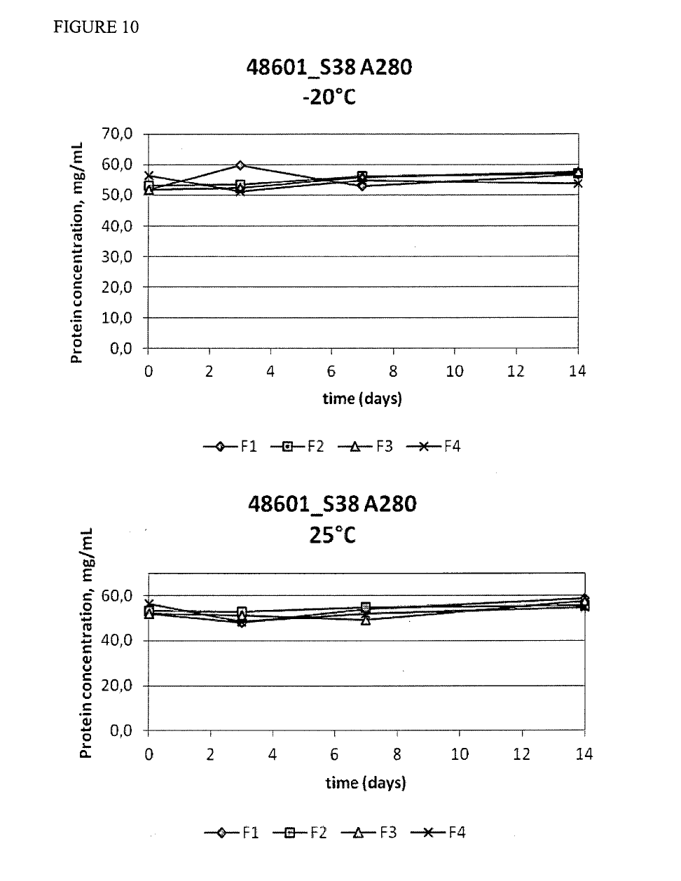

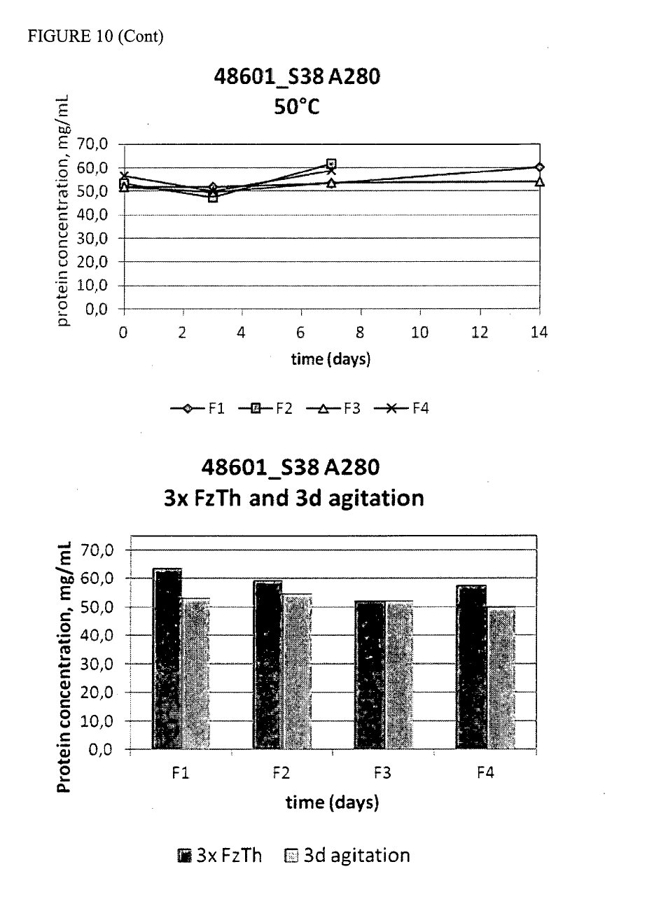

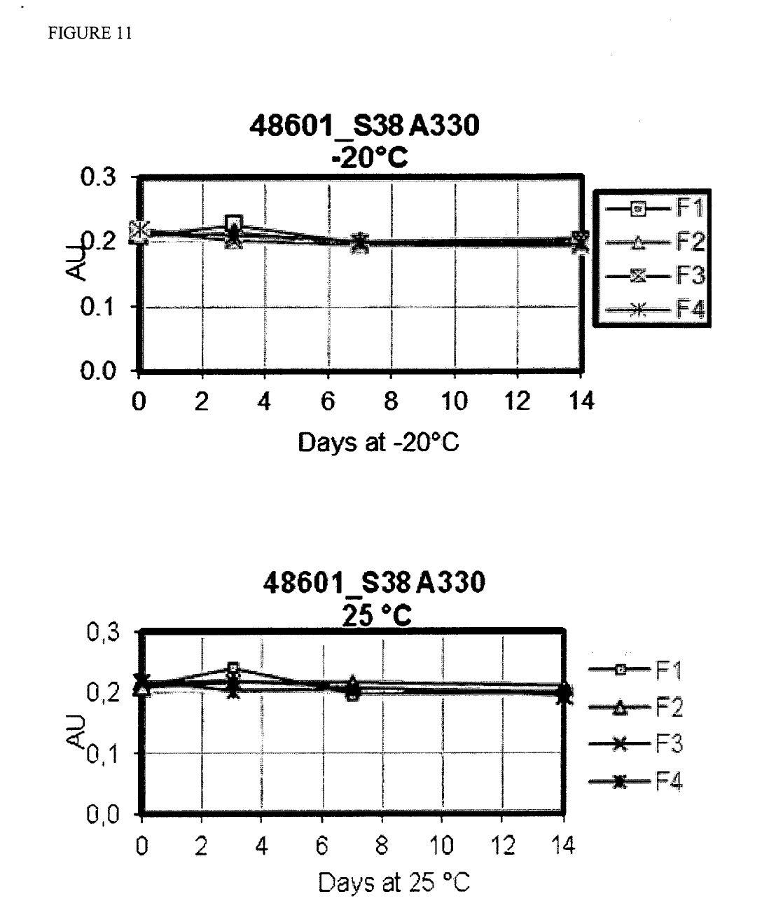

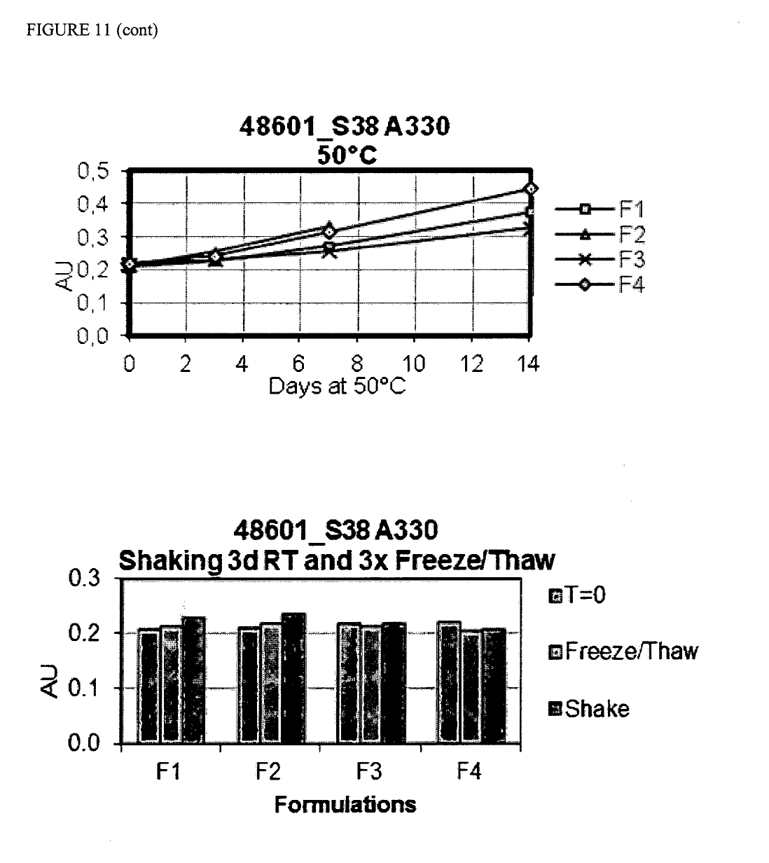

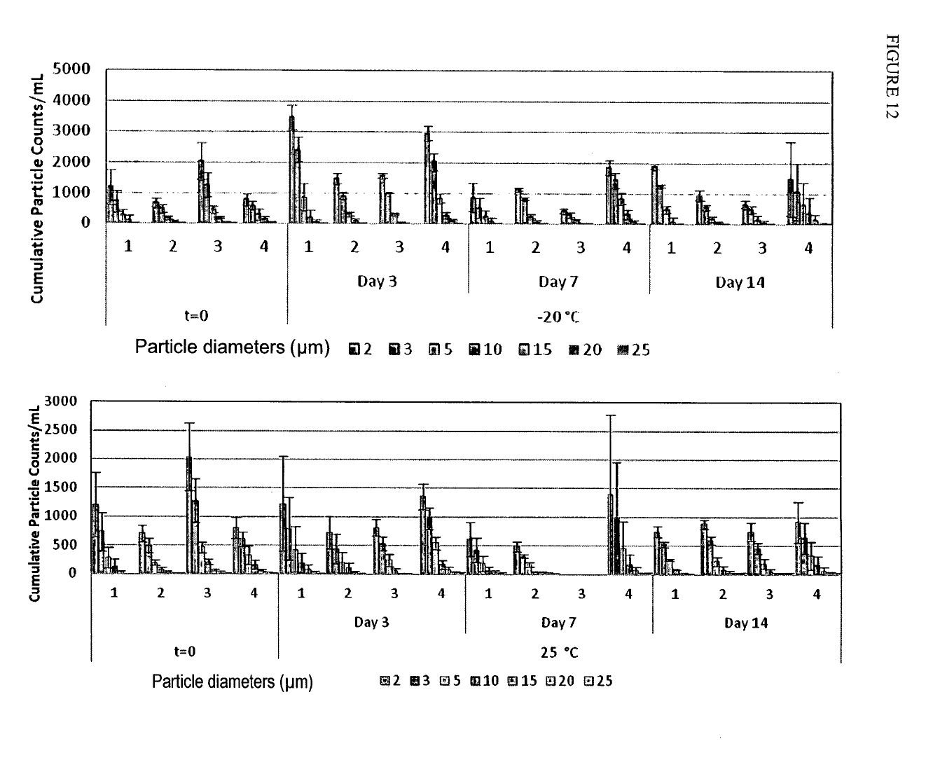

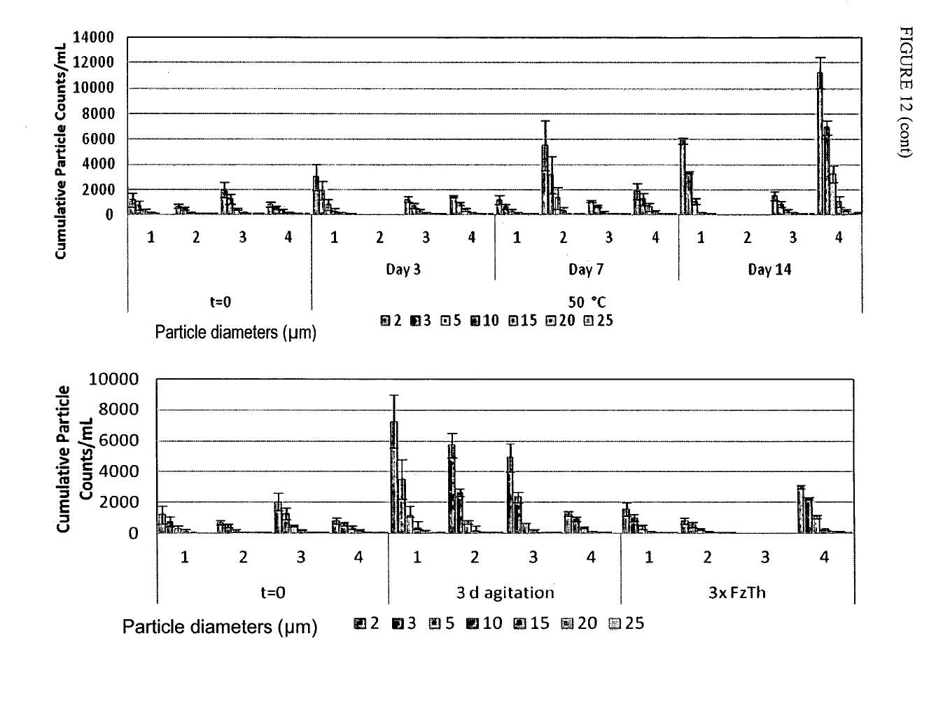

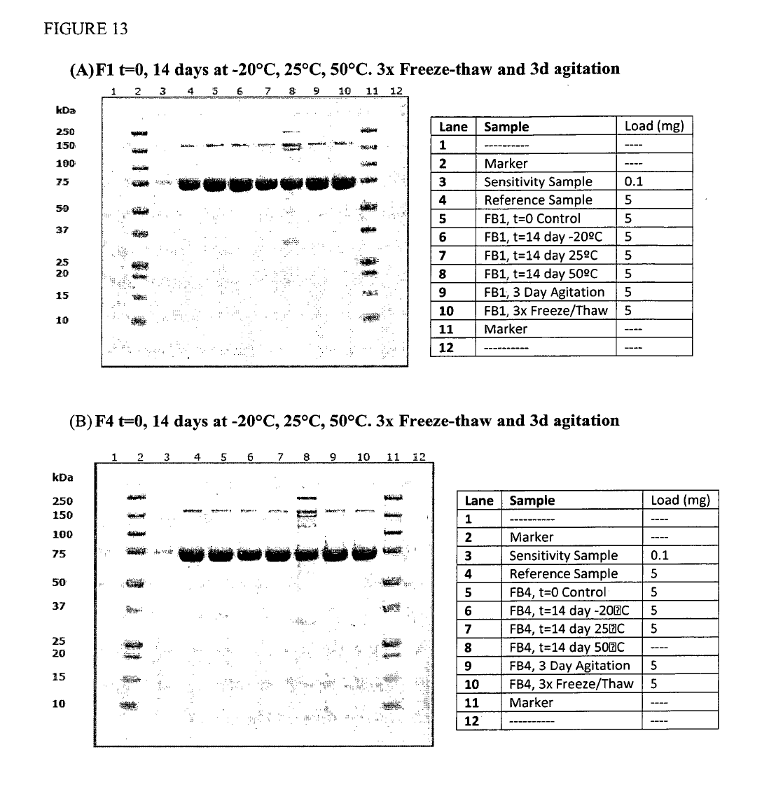

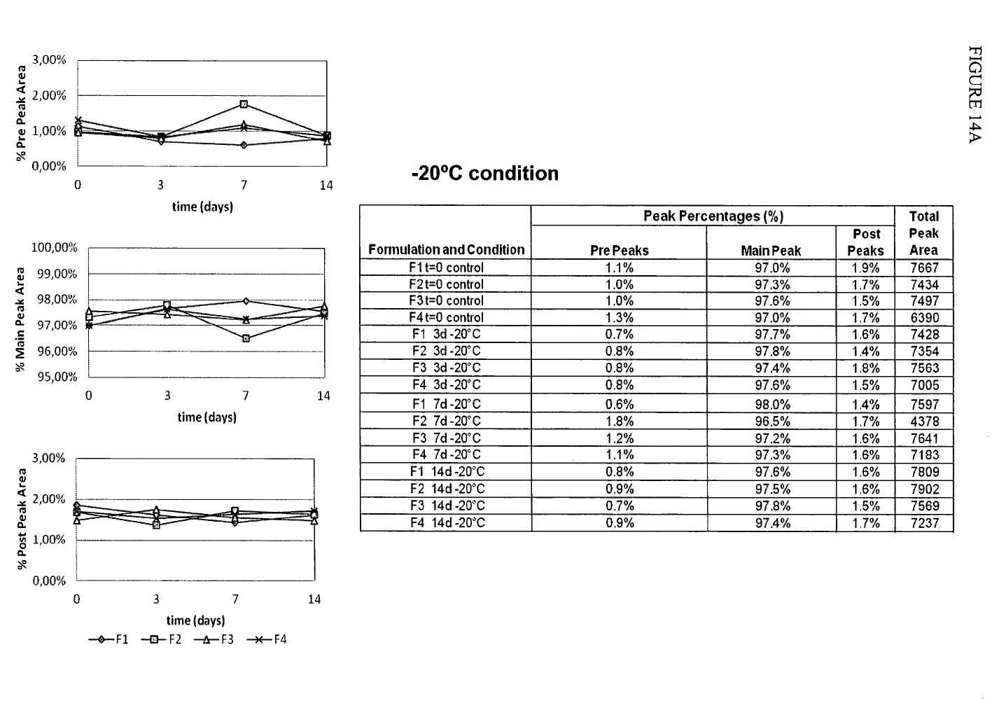

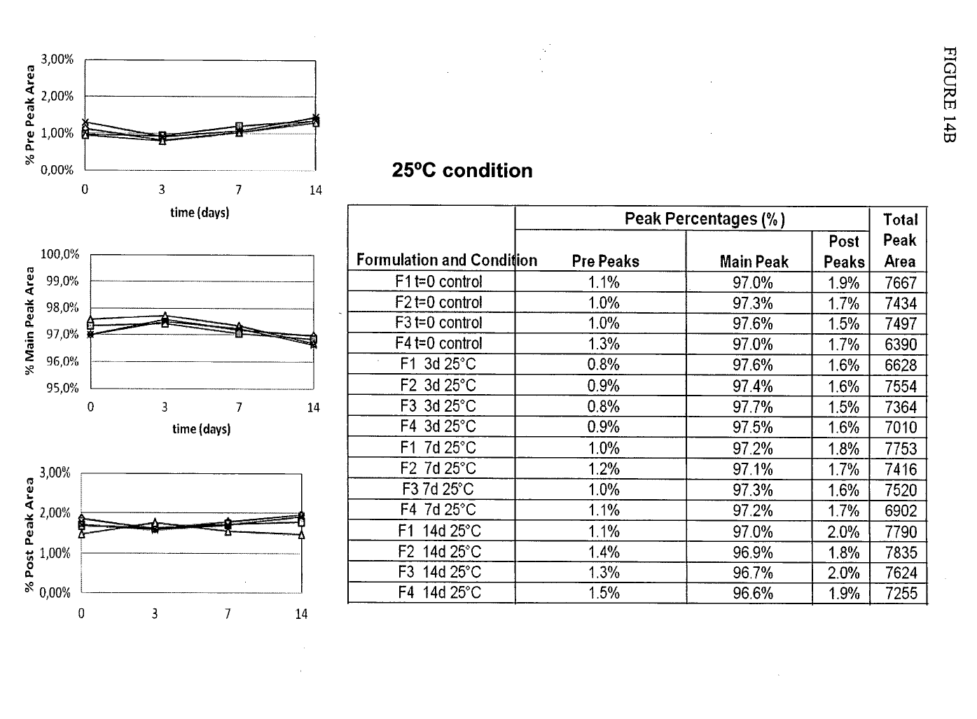

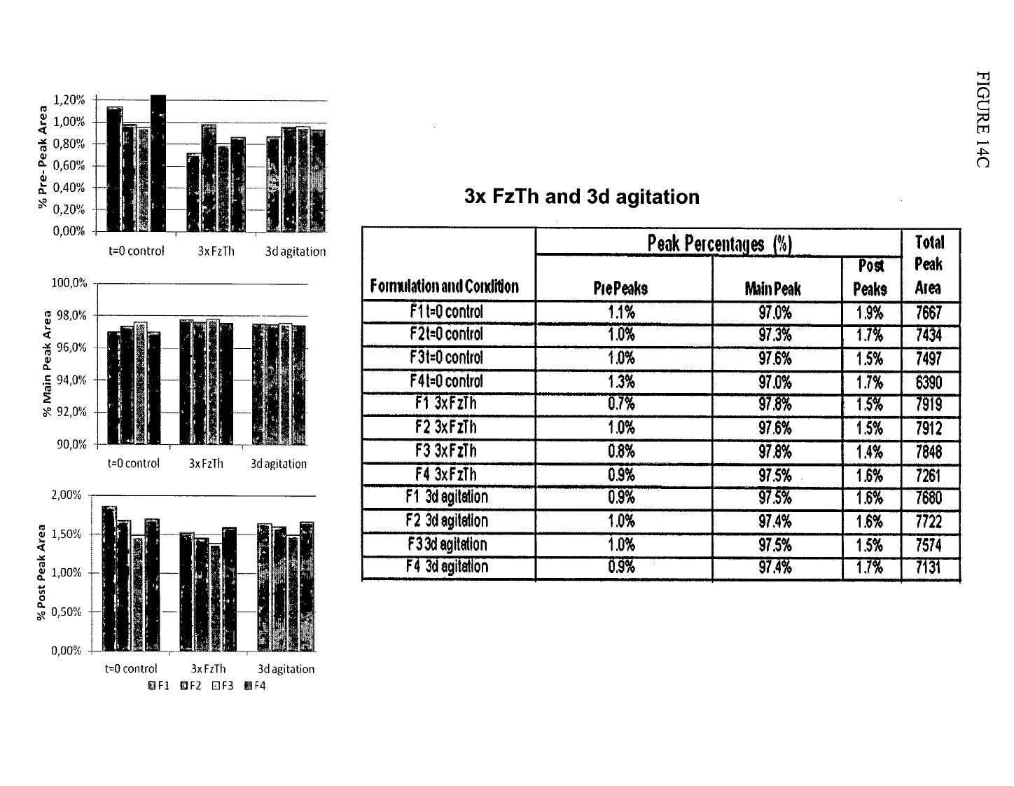

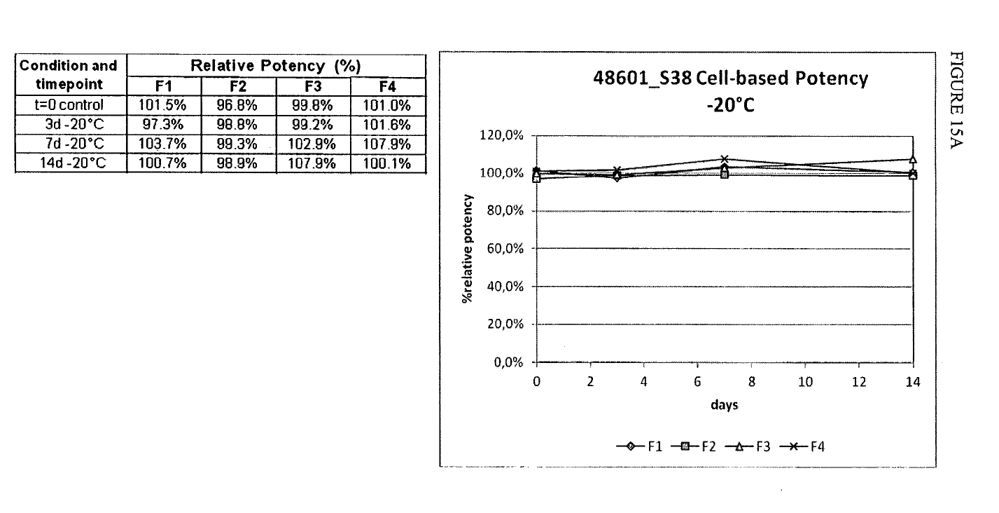

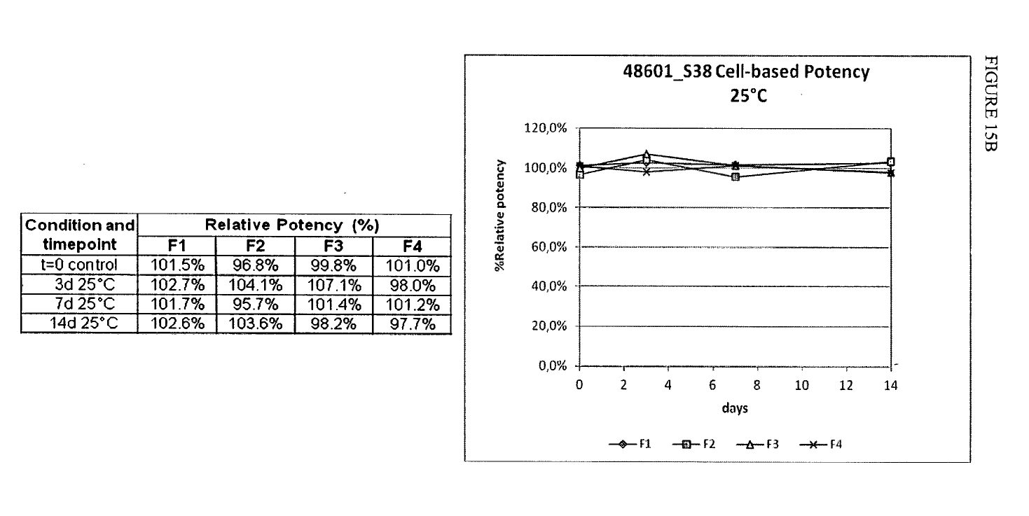

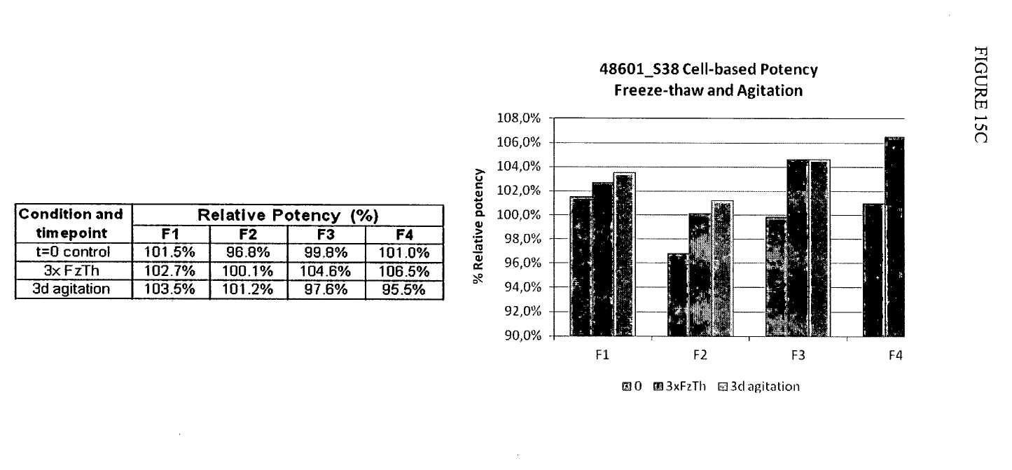

FIG. 8 shows a graph including the analysis of a cell based potency assay (% of relative potency, as compared to potency of the reference standard) in all formulations for all conditions: −20° C. (8A), 25° C. (8B), 50° C. (8C), 3 times freezing/thawing and 3 days in agitation (8D) at all time points.

Differences in potency (as compared to potency of the reference standard) were detected in all formulations at the 50° C. condition, with all test samples losing potency as early as 3 days and increasing significantly by 14 days storage at 50° C.

F3 demonstrates the highest potency after 14 days at 50° C., with 42.2% relative potency remaining.

Relative potencies for all formulations remained close to 100% at −20° C., 25° C. and 50° C. in addition to conditions of freeze-thaw and RT agitation.

FIG. 8E shows a graph including the analysis of a cell based potency assay (% of relative potency, as compared to potency of the reference standard) in formulation F3 for the following conditions: −20° C., 2-8° C., 25° C. at timepoints t=0, t=1 month, t=3 months and t=6 months, and after 1×, 2× and 4× freezing/thawing at −20° C./25° C. The data table is also provided next to the figure.

The formulation F3 at all conditions up to 6 months and after 4 cycles of freeze-thaw at −20° C./25° C. demonstrates % relative potencies which are comparable to the reference standard and remain within the assay variability (≦20%). The lowest % relative potency value (89.5%) was measured for F3 after 3 months at 25° C.

FIG. 8F shows a graph including the analysis of a cell based potency assay (% of relative potency, as compared to potency of the reference standard) in formulations F1, F3, F5, F6 and F8 after 3 month (and F3 after 6 months) at −20° C., 2-8° C., 25° C. and after 4× freezing/thawing at −20° C./25° C., compared to Innovator after 3 months at 25° C. The data table is also provided next to the figure.

No significant differences in % relative potency are observed between F1, F3, F5, and F8 compared to Innovator at all conditions. All samples had relative potencies which were comparable to the reference standard. F6 after 3 months at 25° C. had no remaining potency.

All samples had relative potencies which were comparable to the reference standard.

Overall summary

| | | | | SEC | | | | | | CDC Bioassay | |
|---|---|---|---|---|---|---|---|---|---|---|---|
| | Protein % | | sub visible | % HMW | | % Main | | % LMW post- | | % relative | |
| | Recovery | Turbidity | particulates | pre-peak | | Peak | | peak | | potency | |
| | After | (A330) | (HIAC) 25° C. | | t = 3 mo | | t = 3 mo | | t = 3 mo | | t = 3 mo |
| Formulation | Dialysis | 25° C. 3 mo | 3 mo | t = 0 | (25° C.) | t = 0 | (25° C.) | t = 0 | (25° C.) | t = 0 | (25° C.) |
| 1 | 92.6% | low | no change | 0.8% | 2.0% | 97.9% | 94.6% | 1.4% | 3.4% | 97.9% | 95.6% |
| Innovator | n/a | lowest | no change | 3.4% | 4.6% | 95.0% | 91.6% | 1.6% | 0.0% | 95.1% | 86.3% |
| 3 | 87.4% | high | no change | 1.0% | 2.7% | 96.7% | 94.2% | 2.2% | 3.1% | 98.1% | 89.5% |
| 5 | 85.5% | high | no change | 0.7% | 2.6% | 98.1% | 93.8% | 1.2% | 3.7% | 94.3% | 100.3% |
| 6 | 91.1% | highest | no change | 0.8% | 0.1% | 97.9% | 0.6% | 1.3% | 99.3% | 96.9% | 1.1% |
| 8 | 98.9% | high | no change | 0.8% | 2.3% | 98.0% | 93.9% | 1.3% | 3.9% | 100.0% | 96.7% |

Formulations F5 (50 mM Na phosphate, 90 mM NaCl, 34 mg/mL Sucrose, pH 6.3) and F8 (50 mM Succinate/NaOH, 90 mM NaCl, 10 mg/mL Sucrose, pH 6.3) were identified as lead formulations based on overall highest stability and relative potency from the analysis performed, and as shown in table above, indicating that F8 performed comparably or better than F1 (Innovator liquid formulation) and also better than F3 and F6 formulations. ITEMS
1. An aqueous composition comprising:
   An isolated polypeptide that is an extracellular ligand-binding portion of a human p75 tumor necrosis factor receptor fused to the Fc region of a human IgG1;
   Salt present at a concentration of from 90 to 130 mM; and
   An excipient selected from the group of trehalose and sucrose or a combination thereof, characterized in that neither arginine nor cysteine are present in the composition.
2. The composition according to item 1 wherein the salt concentration is 105-130 mM.
3. The composition according to any of items 1 or 2, wherein the salt concentration is 125 mM.
4. The composition according to any of items 1 to 3, wherein the salt is sodium chloride.
5. The composition according to any of items 1 to 4 wherein the isolated polypeptide is etanercept.
6. The composition according to any of items 1 to 5, wherein the excipient is trehalose at a concentration of from 20 to 80 mg/mL.
7. The composition according to any of items 1 to 6, wherein the excipient is sucrose present at a concentration of from 5 to 80 mg/mL.
8. The composition according to any of items 1 to 7 wherein the composition further comprises an aqueous buffer.
9. The composition according to item 8, wherein the aqueous buffer is sodium phosphate, potassium phosphate, sodium or potassium citrate, succinic acid, maleic acid, ammonium acetate, tris-(hydroxymethyl)-aminomethane (tris), acetate, diethanolamine, histidine or a combination thereof.
10. The composition according to any of items 8 or 9, wherein the aqueous buffer is present at a concentration of 20 mM to 100 mM.
11. The composition according to any of items 1 to 10 further comprising one or more excipients.
12. The composition of item 11, wherein the excipient is lactose, glycerol, xylitol, sorbitol, mannitol, maltose, inositol, glucose, bovine serum albumin, human serum albumin, recombinant hemagglutinin, dextran, polyvinyl alcohol, hydroxypropyl methylcellulose (HPMC), polyethylenimine, gelatine, polyvinylpyrrolidone (PVP), hydroxyethylcellulose (HEC), polyethylene glycol, ethylene glycol, dimethysulfoxide (DMSO), dimethylformamide (DMF), proline, L-serine, glutamic acid, alanine, glycine, lysine, sarcosine, gamma-aminobutyric acid, polysorbate-20, polysorbate-80, sodium dodecyl sulfate, polysorbate, polyoxyethylene copolymer, potassium phosphate, sodium acetate, ammonium sulphate, magnesium sulphate, sodium sulphate, trimethylamine N-oxide, betaine, zinc ions, copper ions, calcium ions, manganese ions, magnesium ions, 3-[(3-cholamidepropyl)-dimethylammonio]-1-propanesulfate, sucrose monolaurate or a combination thereof.
13. The composition according to any of items 1 to 12, wherein the pH of the composition is from pH 6.0 to pH 7.0.
14. The composition according to any of items 1 to 13 comprising 50 mg/mL of etanercept, 25 mM sodium phosphate buffer, 10 mg/mL sucrose, 125 mM sodium chloride, wherein the pH of the composition is 6.3.
15. The composition according to any of items 1 to 13 comprising 50 mg/mL of etanercept, 50 mM sodium phosphate buffer, 60 mg/mL trehalose dihydrate, 0.1% Polysorbate 20, wherein the pH of the composition is pH 6.2.
16. The composition according to any of items 1 to 13, comprising 50 mg/mL of etanercept, 25 mM sodium phosphate buffer, 90 mM sodium chloride, 24 mg/mL sucrose, wherein the pH of the composition is pH 6.3.
17. The composition according to any of items 1 to 13, comprising 50 mg/mL of etanercept, 25 mM sodium phosphate buffer, 90 mM sodium chloride, 10 mg/mL sucrose, 5 mg/mL glycine, wherein the pH of the composition is pH 6.3.
18. The composition according to any of items 1 to 13, comprising 50 mg/mL of etanercept, 22 mM succinate, 90 mM NaCl, 10 mg/mL Sucrose, wherein the pH of the composition is pH 6.3.

### Second Aspect of the Present Invention

A second aspect of the present invention relates to aqueous stable pharmaceutical compositions free of some selected amino acids and some selected salts suitable for storage of polypeptides that contain TNFR:Fc.

The second aspect of the present invention is based on the finding that an aqueous formulation according to the technical features disclosed below can result in an increase of stability of the protein at high temperatures, above 5° C.

Therefore, the second aspect of the present invention relates to an aqueous composition comprising:
an isolated polypeptide that is an extracellular ligand-binding portion of a human p75 tumor necrosis factor receptor fused to the Fc region of a human IgG1;
a monosaccharide or disaccharide;
an aqueous buffer,
   characterized in that said composition neither contains arginine, nor cysteine, nor a salt selected from sodium chloride, potassium chloride, sodium citrate, magnesium sulphate, calcium chloride, sodium hypochlorite, sodium nitrate, mercury sulphide, sodium chromate and magnesium dioxide.

### Brief Description of the Drawings

FIG. 9 shows a bar chart with measures of pH and osmolality at initial time.

FIG. 10 shows the protein concentration measures (Absorbance at 280 nm) at all times (from 0 to 14 days) and conditions (−20° C., 25° C., 50° C., 3 times freezing/thawing and 3 days in agitation).

FIG. 11 shows turbidity measures (Absorbance at 330 nm) at all times (from 0 to 14 days) and conditions (−20° C., 25° C., 50° C., 3 times freezing/thawing and 3 days in agitation).

FIG. 12 shows sub-visible particle analysis by HIAC measured at all conditions: −20° C., 25° C., 50° C., 3 times freezing/thawing and 3 days in agitation using the Standards-Duke Scientific Count Cal.

FIG. 13 shows SDS-PAGE gels stained with Coomassie incubated at all conditions: −20° C., 25° C., 50° C., 3 times freezing/thawing and 3 days in agitation at times 0 and 14 days. In (A), F1 sample and in (B) F4 sample.

FIG. 14 shows the chromatograms of size exclusion HPLC in all formulations for all conditions: −20° C. (14A), 25° C. (14B) and 3 times freezing/thawing and 3 days in agitation (14C) at all timepoints. The peak percentages have been measured and represented in the tables.

FIG. 15 shows a graph including the analysis of a cell based potency assay (% of relative potency, as compared to potency of the reference standard) in all formulations for all conditions: −20° C. (15A), 25° C. (15B), 3 times freezing/thawing and 3 days in agitation (15C) at all timepoints.

### Detailed Description of the Invention

The present invention relates to an aqueous composition comprising:
an isolated polypeptide that is an extracellular ligand-binding portion of a human p75 tumor necrosis factor receptor fused to the Fc region of a human IgG1;
a monosaccharide or disaccharide;
an aqueous buffer,
   characterized in that said composition neither contains arginine, nor cysteine, nor a salt selected from sodium chloride, potassium chloride, sodium citrate, magnesium sulphate, calcium chloride, sodium hypochlorite, sodium nitrate, mercury sulphide, sodium chromate and magnesium dioxide.

As used in this second aspect of the present invention, the term “composition” or “compositions” may refer to a formulation(s) comprising a polypeptide prepared such that it is suitable for injection and/or administration into an individual in need thereof. A “composition” may also be referred to as a “pharmaceutical composition.” In certain embodiments, the compositions provided herein are substantially sterile and do not contain any agents that are unduly toxic or infectious to the recipient. Further, as used in this second aspect of the present invention, a solution or aqueous composition may mean a fluid (liquid) preparation that contains one or more chemical substances dissolved in a suitable solvent (e.g., water and/or other solvent, e.g., organic solvent) or mixture of mutually miscible solvents. Further, as used herein, the term “about” means the indicated value±2% of its value, preferably the term “about” means exactly the indicated value (±0%).

Note that although the composition according to this second aspect of the present invention does not comprise arginine or cysteine alone or added to the composition, the polypeptide itself can contain arginine or cysteine amino acid residues in its chain.

In certain embodiments, the expressed Fc domain containing polypeptide is purified by any standard method. When the Fc domain containing polypeptide is produced intracellularly, the particulate debris is removed, for example, by centrifugation or ultrafiltration. When the polypeptide is secreted into the medium, supernatants from such expression systems can be first concentrated using standard polypeptide concentration filters. Protease inhibitors can also be added to inhibit proteolysis and antibiotics can be included to prevent the growth of microorganisms. In some embodiments, the Fc domain containing polypeptide are purified using, for example, hydroxyapatite chromatography, gel electrophoresis, dialysis, and affinity chromatography, and/or any combination of purification techniques known or yet to discovered. For example, protein A can be used to purify Fc domain containing polypeptides that are based on human gamma 1, gamma 2, or gamma 4 heavy chains (Lindmark et al., 1983, J. Immunol. Meth. 62: 1-13).

Other techniques for polypeptide purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation can also be utilized depending on the needs. Other polypeptide purification techniques can be used.

In a preferred embodiment of this second aspect of the present invention, the isolated polypeptide is etanercept. The Fc component of etanercept contains the constant heavy 2 (CH2) domain, the constant heavy 3 (CH3) domain and hinge region, but not the constant heavy 1 (CH1) domain of human IgG1. Etanercept may be produced by recombinant DNA technology in a Chinese hamster ovary (CHO) mammalian cell expression system. It consists of 934 amino acids and has an apparent molecular weight of/approximately 150 kilodaltons (Physicians' Desk Reference, 2002, Medical Economics Company Inc.).

The concentration of the isolated polypeptide is preferably from 10 to 100 mg/mL, more preferably between 20 and 60 mg/mL and even more preferably the concentration is about 25 mg/mL or about 50 mg/mL.

In another preferred embodiment of this second aspect of the present invention, the monosaccharide or disaccharide is selected from trehalose and sucrose. Preferably, the trehalose is present at a concentration from 20 to 80 mg/mL, more preferably from 40 to 60 mg/mL and even more preferably 60 mg/mL and preferably in the form of trehalose dihydrate. Preferably, the sucrose is present at a concentration from 10 to 80 mg/mL, more preferably from 40 to 60 mg/mL and even more preferably 60 mg/mL. In another preferred embodiment of this second aspect of the present invention, the excipient is a combination between sucrose and trehalose.

In another preferred embodiment of this second aspect of the present invention, the aqueous buffer of the present composition is selected from sodium phosphate, potassium phosphate, sodium or potassium citrate, maleic acid, ammonium acetate, tris-(hydroxymethyl)-aminomethane (tris), acetate, diethanolamine and from a combination thereof. Regardless of the buffer used in the composition, alone or in combination, the concentration thereof is preferably between 20 mM and 150 mM, more preferably the concentration is about 50 mM and the more preferred aqueous buffer is sodium phosphate.

In another embodiment of this second aspect of the present invention, the composition according to the present invention may further comprise one or more excipients. In certain embodiments of this second aspect of the present invention, the concentration of one or more excipients in the composition described herein is about 0.001 to 5 weight percent, while in other embodiments of this second aspect of the present invention, the concentration of one or more excipients is about 0.1 to 2 weight percent. Excipients are well known in the art and are manufactured by known methods and available from commercial suppliers. Preferably, said excipient is lactose, glycerol, xylitol, sorbitol, mannitol, maltose, inositol, glucose, bovine serum albumin, human serum albumin (SA), recombinant hemagglutinin (HA), dextran, polyvinyl alcohol (PVA), hydroxypropyl methylcellulose (HPMC), polyethylenimine, gelatine, polyvinylpyrrolidone (PVP), hydroxyethylcellulose (HEC), polyethylene glycol, ethylene glycol, dimethysulfoxide (DMSO), dimethylformamide (DMF), proline, L-serine, glutamic acid, alanine, glycine, lysine, sarcosine, gamma-aminobutyric acid, polysorbate 20, polysorbate 80, sodium dodecyl sulfate (SDS), polysorbate, polyoxyethylene copolymer, potassium phosphate, sodium acetate, ammonium sulphate, magnesium sulphate, sodium sulphate, trimethylamine N-oxide, betaine, zinc ions, copper ions, calcium ions, manganese ions, magnesium ions, 3-[(3-cholamidepropyl)-dimethylammonio]-1-propanesulfate (CHAPS), sucrose monolaurate or a combination thereof. In a more preferred embodiment, the excipient is polysorbate 20 and in an even more preferred embodiment the polysorbate 20 is present at a concentration of 0.1%.

In another preferred embodiment of this second aspect of the present invention, the pH of the composition is from pH 6.0 to pH 7.0, being possible any pH selected from 6.1, 6.2, 6.3, 6.4, 6.5, 6.6, 6.7, 6.8 and 6.9. In a more preferred embodiment, the pH of the composition is 6.2.

In a particular embodiment of this second aspect of the present invention, the composition comprises 50 mg/mL of etanercept, 50 mM sodium phosphate buffer, 60 mg/mL trehalose dihydrate, wherein the pH of the composition is pH 6.2.

In a particular embodiment of this second aspect of the present invention, the composition comprises 50 mg/mL of etanercept, 50 mM sodium phosphate buffer, 60 mg/mL trehalose dihydrate, 0.1% Polysorbate 20, wherein the pH of the composition is pH 6.2.

In a particular embodiment of this second aspect of the present invention, the composition comprises 50 mg/mL of etanercept, 50 mM sodium phosphate buffer, 60 mg/mL sucrose, wherein the pH of the composition is pH 6.2.

In a particular embodiment of this second aspect of the present invention, the composition comprises 50 mg/mL of etanercept, 50 mM sodium phosphate buffer, 60 mg/mL sucrose, 0.1% Polysorbate 20, wherein the pH of the composition is pH 6.2.

The compositions disclosed in this second aspect of the present invention can be administered parenterally, e.g. subcutaneously, intramuscularly, intravenously, intraperitoneal, intracerebrospinal, intraarticular, intrasynovial and/or intrathecal.

The therapeutic effect of the isolated polypeptide comprised in the compositions according to this second aspect of the present invention are known in the art and includes, but not limited thereto, treating rheumatoid arthritis, psoriatic arthritis, ankylosing spondylitis, granulomatosis, Crohn's disease, chronic obstructive pulmonary disease, hepatitis C, endometriosis, asthma, cachexia, psoriasis or atopic dermatitis, or other inflammatory or autoimmune-related illness, disorder, or condition. The compositions may be administered in an amount sufficient to treat (alleviate symptoms, halt or slow progression of) the disorder (e.g., a therapeutically effective amount).

The following examples serve to illustrate the second aspect of the present invention and should not be construed as limiting the scope thereof.

### Examples of this Second Aspect of the Present Invention

### Preparation of Compositions

The following compositions were prepared by simple mixing:

### Source Material:

Engineering Run Material containing 62.5 mg/mL of etanercept, 1.2 mg/mL Tris, 40 mg/mL Mannitol, 10 mg/mL Sucrose, pH 7.4. Stored at −20° C.

Reference Formulation (Named from Herein as “Enbrel”):

A lot of Enbrel® commercial formulation is used as a control sample. The commercial formulation contains 50 mg/mL etanercept, 25 mM Na phosphate, 25 mM Arginine, 100 mM NaC, 10 mg/mL Sucrose, pH 6.3).

### Candidate Formulations:

F1: Etanercept in the same formulation as Enbrel formulation as internal control (50.9 mg/mL etanercept, 25 mM Na phosphate, 25 mM Arginine, 100 mM NaCl, 10 mg/mL Sucrose, pH 6.3)
F2: Etanercept in aqueous formulation (49.4 mg/mL etanercept, 25 mM Na phosphate, 100 mM NaCl, 10 mg/mL Sucrose, pH 6.3)
F3: Etanercept in aqueous formulation (49.5 mg/mL etanercept, 25 mM Na phosphate, 125 mM NaCl, 10 mg/mL Sucrose, pH 6.3)
F4: Etanercept in aqueous formulation (50.9 mg/mL etanercept, 50 mM Na phosphate, 60 mg/mL Trehalose dihydrate, pH 62, 0.1% Polysorbate 20)

In some experiments, a commercial lot of Enbrel® has been also used as a reference (see above).

### Example 1

### Intrinsic Protein Fluorescence Emission Spectra and Static Light Scattering

Intrinsic protein fluorescence emission spectra, excited at 266 nm, were acquired as well as static light scattering data at both 266 and 473 nm. Each sample was loaded into a micro-cuvette array (MCA) and placed into the Optim 1000 to elucidate differences in colloidal and conformational stabilities. In this study the temperature for thermal ramp experiments was increased from 15 to 95° C. in 1° C. steps, and samples were held at each temperature for 60 seconds to allow thermal equilibration. In the isothermal experiment, the temperature was held at 62° C. and samples were measured with 200 repeats with a 60 second hold between measurements.

The time during which the sample is illuminated with the 266 and 473 nm laser sources is referred to as the exposure time. The choice of exposure time depends on a number of factors, such as how strong the fluorescence emission is and how susceptible the sample is to photobleaching. In the case of all of these samples, an exposure time of 1 second was used.

Along with changing the exposure time it is possible to change the size of a physical slit which controls the amount of light which enters the detector. Increasing the size of this opening increases the fluorescence signal measured, but decreases the spectral resolution of the instrument.

The analyses performed by the Optim 1000 comprise two sequential levels, primary and secondary. The Optim 1000 software provides automated primary and secondary analysis. As with any automated data fitting software, sensible care must be taken to ensure that the input data is of good quality so that the automated functions return reliable results. All the results have been checked manually by a trained analyst.

The primary analysis extracts spectral parameters from the raw fluorescence emission and light scattering data:
Optim can use mathematical functions to provide primary level information such as expectation wavelength (also called the barycentric mean) which is becoming more commonly used in the scientific literature. This looks at the average emission wavelength (or centre of mass), and is a good approach to smooth out any noise in spectral data.
Scattered light intensity is calculated from the integrated intensity between 260 and 270 nm (the Rayleigh scattered UV excitation light). Scattering efficiency is very dependent on wavelength, so the shorter it is the more efficiently that light is scattered by molecules in the solution. The scattering of the 266 nm laser is a very sensitive probe to small changes in mean molecular mass.

In this study, the ratio of fluorescence intensity between 350 and 330 nm has been used to study the thermal unfolding of the antibodies and the scattered light intensity from the 266 nm and 473 am lasers was used to measure thermally induced sample aggregation.

Secondary analysis takes the parameters from the primary analyses and determines the melting temperature “Tₘ” and aggregation onset temperature “T_{agg}” of the sample, if these exist. The melting temperature is determined as the inflection point in the primary data plotted as a function of temperature.

The onset of aggregation temperature is determined as the temperature at which the scattered light intensity increases above a threshold value relative to the noise in the data. From the lowest temperature measured, each scattered intensity value measured is added to a dataset of all previously measured values. At each point, as the analysis progresses, a linear fit is applied and the goodness of the fit determined. If the data deviates significantly from a straight line (where the significance is determined by the noise in the data) then this is defined as the temperature of the onset of aggregation. If it doesn't then the algorithm proceeds to the next point in the dataset and once again tests for this deviation. This method has been tested on a variety of proteins and conditions and is robust. In extreme situations where large aggregates form and precipitate, the light scattering signal can actually fall if the particles in suspension leave the focal volume of the incident laser. However, the initial onset is detected reproducibly despite any precipitation which occurs afterward.

In the case of all static light scattering data, all points have been included regardless of whether the sample appeared to precipitate out of solution. The same sample in different repeated experiments will sometimes precipitate and sometimes not, but in each case the start of the aggregation process is reproducible.

### Conclusions

Both the T_{agg} and Tₒₙₛₑₜ data between all samples were found to be very similar.
In F1 buffer the product was found to have a Tₒₙₛₑₜ of fluorescence of 63.7±0.3° C. and a T_{agg} of 66.8±0.3° C.
In F2 buffer the product was found to have a Tₒₙₛₑₜ of fluorescence of 63.2±0.1° C. and a T_{agg} of 65.9±0.1° C.
In F3 buffer the product was found to have a Tₒₙₛₑₜ of fluorescence of 63.4±0.3° C. and a T_{agg} of 65.6±0.4° C.
In F4 buffer the product was found to have a Tₒₙₛₑₜ of fluorescence of 63.3±0.1° C. and a T_{agg} of 64.8±0.1° C.
Enbrel innovator itself was found to have a Tₒₙₛₑₜ of fluorescence of 63.4±0.1° C. and a T_{agg} of 65.6±0.1° C.

The data therefore indicates a high degree of similarity in both colloidal and conformational stability between all samples.

The Tₒₙₛₑₜ values found for fluorescence were between 63.2 and 63.7° C. with a mean of 63.4° C. and a relatively low standard deviation of 0.3° C., indicating a high degree of comparability between the five samples (F1 to F4 and Enbrel-liquid formulation).

F4 formulation, as indicated in all experiments, seems to be very similar in terms of conformational and colloidal stability conformationally to the Enbrel liquid formulation.

### Example 2

### Short Stress Stability Study

### Approach

A short-term (2-week) stability study was performed in order to evaluate possible formulations prior to execution of a longer-term study.

Four formulations were tested:

| | |
|---|---|
| F1 formulation | 25 mM Na phosphate, 25 mM Arginine, 100 mM |
| | NaCl, 10 mg/mL Sucrose, pH 6.3 |
| F2 formulation | 25 mM Na phosphate, 100 mM NaCl, 10 mg/mL |
| | Sucrose, pH 6.3 |
| F3 formulation | 25 mM Na phosphate, 125 mM NaCl, 10 mg/mL |
| | Sucrose, pH 6.3 |
| F4 formulation | 50 mM Na phosphate, 60 mg/mL Trehalose |
| | dihydrate, pH 6.2, 0.1% Polysorbate 20 |

The stability of each formulation at t=0, 3, 7 and 14 days was assessed, following exposure to two elevated temperatures (25° C. and 50° C.) and one real-time temperature, in addition to agitation and freeze-thaw stress.

A panel of 8 analytical assays was employed to assess the stability of each formulation.
pH (t=0 only)
Osmolality (t=0 only)
Protein concentration (A280 nm)
Turbidity (A330 nm)
HIAC
SDS-PAGE reduced (coomassie blue stain)
Size Exclusion-HPLC
Cell-based potency

### pH and Osmolality

FIG. 9 shows a bar chart with measures of pH and osmolality at initial time. These values measured for all formulations were within range of target pH or theoretical osmolality value prior to setting up the samples at each of the conditions.

### Protein Concentration/A280

FIG. 10 shows the protein concentration measures (Absorbance at 280 nm) at all times (from 0 to 14 days) and conditions (−20° C., 25° C., 50° C., 3 times freezing/thawing (3×FzTh) and 3 days in agitation). The data obtained remained within range of target value and within variability of the assay for all samples at all timepoints and conditions.

### Turbidity/A330

FIG. 11 shows turbidity measures (Absorbance at 330 nm) at all times (from 0 to 14 days) and conditions (−20° C., 25° C., 50° C., 3 times freezing/thawing (3×FzTh) and 3 days in agitation). According to the results, significant increases in turbidity were detected at the 50° C. condition, with F3 presenting the lowest increase over time. No significant changes were observed in any formulation at −20° C., 25° C., freeze-thaw or agitation

### HIAC (Liquid Particle Counter)

### Method:

A HIAC 9703 Liquid Particle Counting System was used for the experiments. The HIAC consists of a sampler, particle counter and Royco sensor. The Royco sensor is capable of sizing and counting particles between 2 μm to 100 μm. The instrument can count particles≦10,000 counts/mL.

### Procedure:

Initially samples were analyzed without dilution, but due to the sample's high viscosity it was determined that they needed to be diluted to obtain a more accurate result.
Samples were brought to room temperature for 1 hr.
Samples were diluted 1:3 in the appropriate formulation buffer, degassed (1.5 hrs) and carefully mixed prior to measurement.
Standards-Duke Scientific Count Cal:System suitability checks are performed with the EZY-Cal 5 μm and 15 μm particle size control standards. The control standards are analyzed at the beginning to verify resolution of the sensor.

FIG. 12 shows sub-visible particle analysis by HIAC measured at all conditions: −20° C., 25° C., 50° C., 3 times freezing/thawing (3×FzTh) and 3 days in agitation using the Standards-Duke Scientific Count Cal.

Significant increases in subvisible particle counts were measured at the 50° C. condition for F1, F2 and F4, with F2 showing the highest increase from as early as 7 days.

No significant changes were observed for any formulation at −20° C., 25° C., 3×FzTh or after 3 d RT agitation.

F4 presented no change in subvisible particle as compared to t=0 control after storage under all conditions and time points.

### SDS-PAGE

FIG. 13 shows SDS-PAGE gels stained with Coomassie incubated at all conditions: −20° C., 25° C., 50° C., 3 times freezing/thawing and 3 days in agitation at times 0 and 14 days. In (A), F1 sample and in (D) F4 sample.

Significant changes observed in all formulations for the 50° C. condition at all timepoints, with day 14 samples showing likely covalently-modified high molecular weight (HMW) species as evidenced by additional HMW bands present (>˜250 kDa) and low molecular weight (LMW) breakdown species (<50 kDa), which were present from as early as 3 days at 50° C. for all formulations.

No changes were observed in any formulation for all other conditions and time points and as compared to the reference standard.

### SE HPLC (Size Exclusion HPLC)

### Conditions:

Column: TSKGel SuperSW3000 4.6×300 mm, 4 μm (Tosoh, 18675) CV=2.5 mL
Column Temp: 25° C.
Mobile Phase: 0.2 M Phosphate Buffer, pH 6.8
Flow Rate: 0.35 mL/min
Runtime: 20 min
Sample Load: 37.6 μg
Auto Sampler Temperature: 4° C.

FIG. 14 shows the chromatograms of size exclusion HPLC in all formulations for the following conditions: −20° C. (14A), 25° C. (14B), 3 times freezing/thawing and 3 days in agitation (14C) at all timepoints. The peak percentages have been measured and represented in the tables.

The 25° C. condition resulted in slight changes for all formulations in both % main peak area and % pre-peak after 7 days, increasing further at 14 days, with F4 demonstrating the highest increase in pre-peak aggregates (0.5%), but this increase is insignificant to be worth considering.

No significant changes were observed in any formulation when exposed to conditions of agitation and freeze-thaw or storage at −20° C. for up to 14 days

### Cell Based Potency Assay

### Approach:

Samples were tested two batches (after t=0 and t=3 d and after t=7 and t=14 d time points)
All the samples were tested in the bioassay once by a single analyst, except the control sample which was tested on each of the six (6) testing days.
Absorbance measurements at A280 nm were taken to determine the accurate concentration of the primary dilutions and subsequent sample dilution
Overall assay performance was acceptable. Three (3) out of 106 dose response curves (from 53 plates) needed to have one well at up to 2 different concentrations masked to meet the well-to-well variability assay criteria
Well-to-well variability % CV≦20%
Assay window (D/A)≧6
R²≧0.98

The relative potency of 47 test samples was measured once and a control was measured six (6) different times. The mean relative potency of the control was 100.2% with 95% CI from 96.9% to 103.6%.
The assay variability (% GCV) for the six independent measurements of the control was 3.2%. The low assay variability of this method demonstrated that the relative potency values of test samples obtained from single measurement was acceptable.
Based on single measurements, the majority of the test samples had relative potencies close to 100% (comparable to that of the reference standard).

### Cell Based Bioassay Results:

FIG. 15 shows a graph including the analysis of a cell based potency assay (% of relative potency, as compared to potency of the reference standard) in all formulations for all conditions: −20° C. (15A), 25° C. (15B), 3 times freezing/thawing and 3 days in agitation (15C) at all timepoints.

As can be seen from FIG. 15, relative potencies for all formulations remained close to 100% at −20° C. and 25° C. in addition to conditions of freeze-thaw and RT agitation.

### Items of the Second Aspect of the Present Invention

1. An aqueous composition comprising:
an isolated polypeptide that is an extracellular ligand-binding portion of a human p75 tumor necrosis factor receptor fused to the Fc region of a human IgG1;
a monosaccharide or disaccharide;
an aqueous buffer,
   characterized in that said composition neither contains arginine, nor cysteine, nor a salt selected from sodium chloride, potassium chloride, sodium citrate, magnesium sulphate, calcium chloride, sodium hypochlorite, sodium nitrate, mercury sulphide, sodium chromate and magnesium dioxide.
   2. The composition according to claim 1 wherein the isolated polypeptide is etanercept.
   3. The composition according to any of items 1 or 2, wherein the monosaccharide or disaccharide is selected from trehalose and sucrose and combinations thereof.
   4. The composition according to item 3, wherein the trehalose is present at a concentration from 20 to 80 mg/mL.
   5. The composition according to item 3, wherein the sucrose is present at a concentration from 10 to 80 mg/mL.
   6. The composition according to any of items 1 to 5, wherein the aqueous buffer is selected from sodium phosphate, potassium phosphate, sodium or potassium citrate, maleic acid, ammonium acetate, tris-(hydroxymethyl)-aminomethane (tris), acetate, diethanolamine or a combination thereof.
   7. The composition according to item 6, wherein the aqueous buffer is present at a concentration of 20 mM to 150 mM.
   8. The composition according to any of items 1 to 7 further comprising one or more excipients.
   9. The composition of item 8, wherein the excipient is lactose, glycerol, xylitol, sorbitol, mannitol, maltose, inositol, glucose, bovine serum albumin, human serum albumin, recombinant hemagglutinin, dextran, polyvinyl alcohol, hydroxypropyl methylcellulose (HPMC), polyethylenimine, gelatine, polyvinlylpyrrolidone (PVP), hydroxyethylcellulose (HEC), polyethylene glycol, ethylene glycol, dimethysulfoxide (DMSO), dimethylformamide (DMF), proline, L-serine, glutamic acid, alanine, glycine, lysine, sarcosine, gamma-aminobutyric acid, polysorbate 20, polysorbate 80, sodium dodecyl sulfate, polysorbate, polyoxyethylene copolymer, potassium phosphate, sodium acetate, ammonium sulphate, magnesium sulphate, sodium sulphate, trimethylamine N-oxide, betaine, zinc ions, copper ions, calcium ions, manganese ions, magnesium ions, 3-[(3-cholamidepropyl)-dimethylammonio]-1-propanesulfate, sucrose monolaurate or a combination thereof.
   10. The composition according to any of items 1 to 9, wherein the pH of the composition is from pH 6.0 to pH 7.0.
   11. The composition according to any of items 1 to 10 comprising 50 mg/mL of etanercept, 50 mM sodium phosphate buffer, 60 mg/mL trehalose dihydrate, wherein the pH of the composition is pH 6.2.
   12. The composition according to any of items 1 to 10 comprising 50 mg/mL of etanercept, 50 mM sodium phosphate buffer, 60 mg/mL sucrose, wherein the pH of the composition is pH 6.2.
   13. The composition according to any of items 1 to 10 comprising 50 mg/mL of etanercept, 50 mM sodium phosphate buffer, 60 mg/mL trehalose dihydrate, 0.1% Polysorbate 20, wherein the pH of the composition is pH 6.2.
   14. The composition according to any of items 1 to 10 comprising 50 mg/mL of etanercept, 50 mM sodium phosphate buffer, 60 mg/mL sucrose, 0.1% Polysorbate 20, wherein the pH of the composition is pH 6.2.

1. An aqueous composition comprising:
an isolated polypeptide that is an extracellular ligand-binding portion of a human p75 tumor necrosis factor receptor fused to the Fc region of a human IgG1;
salt present at a concentration of between 80 and 130 mM;
an aqueous buffer, wherein the aqueous buffer is sodium and/or potassium phosphate buffer and wherein the aqueous buffer is present at a concentration of between 20 and 30 mM; and
an excipient which is sucrose, wherein the concentration of sucrose is between 34 and 80 mg/mL,
characterized in that neither arginine nor cysteine are present in the composition.

2. The composition according to claim 1, wherein the salt concentration is 90 mM.

3. The composition according to claim 1, wherein the salt is sodium chloride.

4. The composition according to claim 1, wherein the isolated polypeptide is etanercept.

5. An aqueous composition comprising:
an isolated polypeptide that is an extracellular ligand-binding portion of a human p75 tumor necrosis factor receptor fused to the Fc region of a human IgG1;
salt present at a concentration of from 80 to 130 mM, wherein the salt is not present at a concentration of 100 mM;
an aqueous buffer, wherein the aqueous buffer is succinate buffer; and
an excipient selected from the group of consisting of trehalose, sucrose, and a combination thereof, wherein no free amino acids are present in the composition.

6. The composition according to claim 5, wherein the salt concentration is 90 mM.

7. The composition according to claim 5, wherein the salt is sodium chloride.

8. The composition according to claim 5, wherein the isolated polypeptide is etanercept.

9. The composition according to claim 5, wherein the excipient is sucrose present at a concentration of from 5 to 80 mg/mL.

10. The composition according to claim 5, wherein the aqueous buffer is present at a concentration of between 15 mM and 100 mM.

11. The composition according to claim 10, wherein the aqueous buffer is present at a concentration of between 20 and 30 mM.

12. The composition according to claim 10, wherein the aqueous buffer is present at a concentration of 50 mM.

13. The composition according to claim 1, further comprising one or more excipients.

14. The composition of claim 13, wherein the excipient is selected from the group consisting of lactose, glycerol, xylitol, sorbitol, mannitol, maltose, inositol, glucose, bovine serum albumin, human serum albumin, recombinant hemagglutinin, dextran, polyvinyl alcohol, hydroxypropyl methylcellulose (HPMC), polyethylenimine, gelatine, polyvinlylpyrrolidone (PVP), hydroxyethylcellulose (HEC), polyethylene glycol, ethylene glycol, dimethysulfoxide (DMSO), dimethylformamide (DMF), proline, L-serine, glutamic acid, alanine, glycine, lysine, sarcosine, gamma-aminobutyric acid, polysorbate-20, polysorbate-80, sodium dodecyl sulfate, polysorbate, polyoxyethylene copolymer, potassium phosphate, sodium acetate, ammonium sulphate, magnesium sulphate, sodium sulphate, trimethylamine N-oxide, betaine, zinc ions, copper ions, calcium ions, manganese ions, magnesium ions, 3-[(3-cholamidepropyl)-dimethylammonio]-1-propanesulfate, sucrose monolaurate or and a combination thereof.

15. The composition according to claim 1, wherein the pH of the composition is between pH 6.0 and pH 7.0.

16. The composition according to claim 5, comprising 50 mg/mL of etanercept, 22 mM succinate, 90 mM NaCl, 10 mg/mL sucrose, wherein the pH of the composition is pH 6.3.

17. The composition according to claim 1, comprising 50 mg/mL of etanercept, 25 mM sodium phosphate buffer, 90 mM sodium chloride, 34 mg/mL sucrose, wherein the pH of the composition is pH 6.3.
